(19) United States
(12) Patent Application Publication
(10) Pub. No.: US 2021/0316013 A1
(43) Pub. Date: Oct. 14, 2021

(54) HAEMATOPOIETIC STEM CELL-GENE THERAPY FOR WISKOTT-ALDRICH SYNDROME

(71) Applicant: CSL BEHRING LLC, King of Prussia, PA (US)

(72) Inventors: Cédric Pierre Vonarburg, Bern (CH); Ming Yan, Encino, CA (US); Chi-Lin Lee, Arcadia, CA (US); Chao-Guang Chen, Parkville (AU); Walid Jhan Azar, Elsternwick (AU)

(21) Appl. No.: 17/353,586

(22) Filed: Jun. 21, 2021

Related U.S. Application Data
(63) Continuation of application No. PCT/US2019/068233, filed on Dec. 23, 2019.
(60) Provisional application No. 62/784,508, filed on Dec. 23, 2018.

(51) Int. Cl. A61K 48/00; C12N 15/86; A61K 35/28; C07K 14/47; C12N 15/113

(52) U.S. Cl. CPC A61K 48/005; C12N 15/86; A61K 35/28; C12N 2330/51; C12N 15/1137; C12N 2740/15043; C12N 2310/531; C07K 14/47

(57) ABSTRACT

The present disclosure provides expression vectors comprising at least two nucleic acid sequences, namely a nucleic acid sequence encoding an anti-HPRT RNAi, and a nucleic acid sequence encoding a Wiskott-Aldrich Syndrome protein. In some embodiments, the expression vector is a self-inactivating lentiviral vector. In some embodiments, the Wiskott-Aldrich Syndrome protein is used to alleviate the pathologies associated with Wiskott-Aldrich Syndrome.

### CROSS REFERENCE TO RELATED APPLICATIONS

The present application is a continuation of International Application No. PCT/US2019/068233 filed on Dec. 23, 2019, which application claims the benefit of the filing date of U.S. Provisional Application No. 62/784,508, filed on Dec. 23, 2018, the disclosures of which are hereby incorporated by reference herein in their entireties.

### FIELD OF DISCLOSURE

The present disclosure generally relates to gene therapy and, in particular, hematopoietic stem cells transduced by expression vectors.

### BACKGROUND OF THE DISCLOSURE

Wiskott-Aldrich Syndrome (WAS) is a rare, X-linked primary immunodeficiency (PID) disorder characterized by recurrent infections, small platelets, microthrombocytopenia, eczema, and increased risk of autoimmune manifestations and tumors. Mutations in the Wiskott-Aldrich Syndrome protein (WASP) gene are responsible for Wiskott-Aldrich Syndrome. The gene that encodes the WAS protein is located in the short arm of X chromosome (XP11.22-11.23) and is about 9 kb, including 12 exons, and encoding 502 amino acids. To date, WASP mutations, including missense/nonsense, splicing, small deletions, small insertions, gross deletions, and gross insertions have been identified in patients with Wiskott-Aldrich Syndrome

Wiskott-Aldrich Syndrome protein is a hematopoietic system-specific intracellular signal transduction molecule, which is proline rich, and expressed only in hematopoietic cell lines. Wiskott-Aldrich Syndrome protein is believed to be an important regulator of the actin cytoskeleton found to be expressed in all leukocytes. It is believed to be involved in dynamic cytoskeletal changes, which are essential for multiple cellular functions such as adhesion, migration, phagocytosis, immune synapse formation, and receptor-mediated cellular activation processes (e.g. B and T cell antigen receptors). As a result, both innate and cellular adaptive immunity are believed to be affected in Wiskott-Aldrich Syndrome patients, rendering these patients highly susceptible to infections.

In general, WAS gene mutations that cause absent protein expression result in “classic Wiskott-Aldrich Syndrome.” Reduced Wiskott-Aldrich Syndrome protein expression results in X-linked thrombocytopenia. Wiskott-Aldrich Syndrome protein activating gain-of-function mutations result in X-linked neutropenia. Depending on the mutations within the WAS gene product, there is wide variability of clinical disease. In one study of 154 patients with Wiskott-Aldrich Syndrome, only 30% had the classic presentation with thrombocytopenia, small platelets, eczema, and immunodeficiency; 84% had clinical signs and symptoms of thrombocytopenia, 80% had eczema, 20% had only hematologic abnormalities, and 5% had only infectious manifestations. (see Sullivan, J Pediatr. 1994; 125(6 Pt 1):876-85). Autoimmune disease is common and occurs in up to 40-70% of patients. There is also believed to be a significantly increased risk of lymphoreticular malignancy (10-20%), such as lymphoma, leukemia, and myelodysplasia. Another review of 55 patients with Wiskott-Aldrich Syndrome from a single hospital in France, over a course of 20 years, found autoimmune or inflammatory conditions in 70% of patients, most commonly autoimmune hemolytic anemia.

Wiskott-Aldrich Syndrome was one of the first conditions ever to be successfully treated by allogeneic hematopoietic stem cell transplantation (HSCT) nearly 40 years ago (Galy, Roncarolo et al. 2008, Candotti 2018). Gene therapy approaches for treatment of WAS continue to be reported, including, for example, Aiuti et al. (2013), Science, 341, p. 1233151; Hacein-Bey Abina, et al. (2015) AMA, 313, pp. 1550-1563; Koldej et al. (2013), Human Gene Therapy Clinical Development, Vol 24, pp. 77-85; Wielgosz et al. (2015); Molecular Therapy: Methods & Clinical Development Vol 2, pp. 14063 and Singh et al. (2017), Molecular Therapy: Methods & Clinical Development Vol. 4 pp. 1-16.

It is believed that a bone marrow transplant remains the only proven cure for this disease and the outcome is reasonably good for those patients with HLA-matched donors (only available for less than 20% of patients). Hematopoietic stem cell gene therapy (HSC-GT) offers a new, potentially curative, option for patients lacking a matched donor. Gene therapy offers several potential advantages over allogeneic HSCT. It is theoretically available to all patients and is believed to decrease the risks of graft rejection, and possibly avoiding the risks associated with Graft versus Host Disease (GvHD).

### BRIEF SUMMARY OF THE DISCLOSURE

Gene therapy strategies to modify human stem cells hold great promise for curing many human diseases. One of the problems with gene therapy, however, is obtaining sufficient levels of engraftment. It is believed that the engraftment of gene modified stem cells may be enhanced by engineering stem cells in which hypoxanthine guanine phosphoribosyitransferase (“HPRT”) expression is knocked down, thereby enabling the selection of genetically modified cells by conferring resistance to a guanine analog antimetabolite.

In a first aspect of the present disclosure there is provided an expression vector comprising a first expression control sequence operably linked to a first nucleic acid sequence, the first nucleic acid sequence encoding a shRNA to knockdown HPRT; and a second expression control sequence operably linked to a second nucleic acid sequence, the second nucleic acid sequence encoding a Wiskott-Aldrich Syndrome protein (e.g. a wild-type Wiskott-Aldrich Syndrome protein or a codon-optimized Wiskott-Aldrich Syndrome protein). In some embodiments, the expression vector is a lentiviral expression vector. In some embodiments, the lentiviral expression vector is an integration defective lentiviral vector.

In some embodiments, the shRNA comprises a hairpin loop sequence having SEQ ID NO: 32. In some embodiments, the shRNA has at least 95% sequence identity to the nucleic acid sequence of SEQ ID NO: 26. In some embodiments, the shRNA comprises the sequence of SEQ ID NO: 26. In some embodiments, the shRNA has at least 95% sequence identity to a nucleic acid sequence selected from the group consisting of SEQ ID NO: 23, SEQ ID NO: 24, and SEQ ID NO: 25. In some embodiments, the shRNA comprises the sequence of any one of SEQ ID NO: 23, SEQ ID NO: 24, and SEQ ID NO: 25. In some embodiments, shRNA has at least 95% sequence identity to a nucleic acid sequence selected from the group consisting of SEQ ID NO: 34 and SEQ ID NO: 35. In some embodiments, the shRNA comprises the sequence of any one of SEQ ID NO: 34 and SEQ ID NO: 35. In some embodiments, the shRNA has at least 95% sequence identity to a nucleic acid sequence selected from the group consisting of SEQ ID NO: 21 and SEQ ID NO: 22. In some embodiments, the shRNA comprises the sequence of any one of SEQ ID NO: 21 and SEQ ID NO: 22. In some embodiments, the shRNA has at least 95% sequence identity to the nucleic acid sequence of SEQ ID NO: 36. In some embodiments, the shRNA comprises the sequence of SEQ ID NO: 36.

In some embodiments, the first expression control sequence comprises a Pol III promoter or a Pol II promoter. In some embodiments, the Pol III promoter is 7sk. In some embodiments, the 7sk promoter comprises a nucleic acid sequence having at least 95% sequence identity to that of SEQ ID NO: 28. In some embodiments, the 7sk promoter comprises the nucleic acid sequence of SEQ ID NO: 28. In some embodiments, the 7sk promoter comprises the nucleic acid sequence of SEQ ID NO: 29.

In some embodiments, the second nucleic acid encoding the Wiskott-Aldrich Syndrome protein comprises a sequence having at least 95% identity to any one of SEQ ID NOS: 1, 2, 3, and 4. In some embodiments, the second nucleic acid encoding the Wiskott-Aldrich Syndrome protein comprises a sequence having at least 97% identity to any one of SEQ ID NOS: 1, 2, 3, and 4. In some embodiments, the second nucleic acid encoding the Wiskott-Aldrich Syndrome protein comprises a sequence having at least 99% identity to any one of SEQ ID NOS: 1, 2, 3, and 4. In some embodiments, second nucleic acid encoding the Wiskott-Aldrich Syndrome protein comprises a sequence comprising any one of SEQ ID NOS: 1, 2, 3, and 4. In some embodiments, the second nucleic acid encoding the Wiskott-Aldrich Syndrome protein comprises a sequence having at least 95% identity to any one of SEQ ID NOS: 67, 68, and 69. In some embodiments, the second nucleic acid encoding the Wiskott-Aldrich Syndrome protein comprises a sequence having at least 97% identity to any one of SEQ ID NOS: 67, 68, and 69. In some embodiments, the second nucleic acid encoding the Wiskott-Aldrich Syndrome protein comprises a sequence having at least 99% identity to any one of SEQ ID NOS: 67, 68, and 69. In some embodiments, second nucleic acid encoding the Wiskott-Aldrich Syndrome protein comprises a sequence comprising any one of SEQ ID NOS: 67, 68, and 69. In some embodiments, the second expression control sequence comprises an MND promoter. In some embodiments, the MND promoter comprises at least 95% identity to any one of SEQ ID NOS: 7, 8, 9, 10, 11, and 12. In some embodiments, the MND promoter comprises at least 99% identity to any one of SEQ ID NOS: 7, 8, 9, 10, 11, and 12.

In some embodiments, the second nucleic acid sequence encodes for a peptide having an amino acid sequence having at least 95% identity to any one of SEQS ID NOS: 5 and 6; and the first nucleic acid sequence encodes a nucleic acid molecule having at least 95% identity to SEQ ID NO: 16 or its complement thereof.

In some embodiments, the expression vector further comprises an insulator. In some embodiments, the insulator is selected from the group consisting of a 650cHS4 insulator, a 400cHS4 insulator, and a foamy virus insulator. In some embodiments, the expression vector further comprises an insulator having at least 95% sequence identity to a nucleic acid sequence selected from the group consisting of SEQ ID NO: 38, SEQ ID NO: 39, and SEQ ID NO: 40. In some embodiments, the expression vector further comprises an insulator having a nucleic acid sequence selected from the group consisting of SEQ ID NO: 38, SEQ ID NO: 39, and SEQ ID NO: 40.

In some embodiments, the first expression control sequence operably linked to the first nucleic acid sequence is located downstream from the second expression control sequence operably linked to the second nucleic acid sequence. In some embodiments, the first expression control sequence operably linked to the first nucleic acid sequence is oriented in the same direction as the second expression control sequence operably linked to the second nucleic acid sequence. In some embodiments, the first expression control sequence operably linked to the first nucleic acid sequence comprises is oriented in a first direction, wherein the second expression control sequence operably linked to the second nucleic acid sequence is oriented in a second direction, and where the first and second directions are opposite.

In some embodiments, the first expression control sequence operably linked to the first nucleic acid sequence is located upstream from the second expression control sequence operably linked to the second nucleic acid sequence. In some embodiments, the first expression control sequence operably linked to the first nucleic acid sequence is oriented in the same direction as the second expression control sequence operably linked to the second nucleic acid sequence. In some embodiments, the first expression control sequence operably linked to the first nucleic acid sequence comprises is oriented in a first direction, wherein the second expression control sequence operably linked to the second nucleic acid sequence is oriented in a second direction, and where the first and second directions are opposite.

In some embodiments, the first expression control sequence operably linked to the first nucleic acid sequence is oriented in a first direction, wherein the second expression control sequence operably linked to the second nucleic acid sequence is oriented in a second direction, and where the first and second directions are opposite. In some embodiments, the first expression control sequence operably linked to the first nucleic acid sequence is located downstream from the second expression control sequence operably linked to the second nucleic acid sequence. In some embodiments, the first expression control sequence operably linked to the first nucleic acid sequence is located upstream from the second expression control sequence operably linked to the second nucleic acid sequence.

In some embodiments, the first expression control sequence operably linked to the first nucleic acid sequence is oriented in the same direction as the second expression control sequence operably linked to the second nucleic acid sequence. In some embodiments, the first expression control sequence operably linked to the first nucleic acid sequence is located downstream from the second expression control sequence operably linked to the second nucleic acid sequence. In some embodiments, the first expression control sequence operably linked to the first nucleic acid sequence is located upstream from the second expression control sequence operably linked to the second nucleic acid sequence.

In a second aspect of the present disclosure there is provided an expression cassette comprising a nucleic acid sequence having at least 90% identity to that of SEQ ID NO: 15. In some embodiments, the expression cassette comprises a nucleic acid sequence having at least 95% identity to that of SEQ ID NO: 15. In some embodiments, the expression cassette comprises a nucleic acid sequence having at least 96% identity to that of SEQ ID NO: 15. In some embodiments, the expression cassette comprises a nucleic acid sequence having at least 97% identity to that of SEQ ID NO: 15. In some embodiments, the expression cassette comprises a nucleic acid sequence having at least 98% identity to that of SEQ ID NO: 15. In some embodiments, the expression cassette comprises a nucleic acid sequence having at least 99% identity to that of SEQ ID NO: 15. In some embodiments, the expression cassette comprises SEQ ID NO: 15.

In a third aspect of the present disclosure there is provided a lentiviral vector comprising an expression cassette comprising a nucleic acid sequence having at least 95% identity to that of SEQ ID NO: 15, and further comprising an insulator selected from the group consisting of a 650cHS4 insulator, a 400cHS4 insulator, and a foamy virus insulator. In some embodiments, the insulator has at least 95% sequence identity to a nucleic acid sequence selected from the group consisting of SEQ ID NO: 38, SEQ ID NO: 39, and SEQ ID NO: 40. In some embodiments, the insulator has a nucleic acid sequence selected from the group consisting of SEQ ID NO: 38, SEQ ID NO: 39, and SEQ ID NO: 40. In some embodiments, the lentiviral vector further comprises a second expression cassette. In some embodiments, the second expression cassette includes a 7sk promoter and a nucleic acid sequence encoding an RNAi to knockdown HPRT. In some embodiments, the second expression cassette comprises a nucleic acid sequence having at least 90% identity to that of SEQ ID NO: 14. In some embodiments, the second expression cassette comprises a nucleic acid sequence having at least 95% identity to that of SEQ ID NO: 14.

In a fourth aspect of the present disclosure there is provided a host cell transduced with an expression vector comprising a first expression control sequence operably linked to a first nucleic acid sequence, the first nucleic acid sequence encoding a shRNA to knockdown HPRT; and a second expression control sequence operably linked to a second nucleic acid sequence, the second nucleic acid sequence encoding a Wiskott-Aldrich Syndrome protein. In some embodiments, the shRNA has at least 95% sequence identity to a nucleic acid sequence selected from the group consisting of SEQ ID NO: 23, SEQ ID NO: 24, and SEQ ID NO: 25. In some embodiments, the Wiskott-Aldrich Syndrome protein is a wild-type Wiskott-Aldrich Syndrome protein. In some embodiments, the Wiskott-Aldrich Syndrome protein is a codon-optimized Wiskott-Aldrich Syndrome protein. In some embodiments, the expression vector is a lentiviral expression vector. In some embodiments, the host cell is substantially HPRT deficient. In some embodiments, the host cell expresses a Wiskott-Aldrich Syndrome protein. In some embodiments, the host cell is formulated with a pharmaceutically acceptable carrier. In some embodiments, the host cell is a hematopoietic stem cell.

In a fifth aspect of the present disclosure there is provided a host cell transduced with a lentiviral vector comprising an expression cassette comprising a nucleic acid sequence having at least 95% identity to that of SEQ ID NO: 15, and further comprising an insulator selected from the group consisting of a 650cHS4 insulator, a 400cHS4 insulator, and a foamy virus insulator. In some embodiments, the lentiviral vector further comprises a second expression cassette. In some embodiments, the host cell is substantially HPRT deficient. In some embodiments, the host cell expresses a Wiskott-Aldrich Syndrome protein. In some embodiments, the host cell is formulated with a pharmaceutically acceptable carrier. In some embodiments, the host cell is a hematopoietic stem cell.

In a sixth aspect of the present disclosure there is provided a host cell which is HPRT deficient and which expresses a peptide having at least 95% sequence identity to an amino acid sequence of any one of SEQ ID NOS: 5 and 6. In some embodiments, the expressed peptide has at least 96% sequence identity to any one of SEQ ID NOS: 5 and 6. In some embodiments, the expressed peptide has at least 97% sequence identity to any one of SEQ ID NOS: 5 and 6. In some embodiments, the expressed peptide has at least 98% sequence identity to any one of SEQ ID NOS: 5 and 6. In some embodiments, the expressed peptide has at least 99% sequence identity to any one of SEQ ID NOS: 5 and 6. In some embodiments, the expressed peptide comprises the amino acid sequence of any one of SEQ ID NOS: 5 and 6. In some embodiments, the host cell is a hematopoietic stem cell.

In a seventh aspect of the present disclosure there is provided a host cell which is HPRT deficient and which expresses a peptide having at least 95% sequence identity an amino acid sequence of any one of SEQ ID NOS: 5 and 6, wherein the host cell is prepared by transducing the host cell with an expression vector comprising a first expression control sequence operably linked to a first nucleic acid sequence, the first nucleic acid sequence encoding a shRNA to knockdown HPRT; and a second expression control sequence operably linked to a second nucleic acid sequence, the second nucleic acid sequence encoding a Wiskott-Aldrich Syndrome protein. In some embodiments, the expressed peptide has at least 96% sequence identity to any one of SEQ ID NOS: 5 and 6. In some embodiments, the expressed peptide has at least 97% sequence identity to any one of SEQ ID NOS: 5 and 6. In some embodiments, the expressed peptide has at least 98% sequence identity to any one of SEQ ID NOS: 5 and 6. In some embodiments, the expressed peptide has at least 99% sequence identity to any one of SEQ ID NOS: 5 and 6. In some embodiments, the expressed peptide comprises the amino acid sequence of any one of SEQ ID NOS: 5 and 6. In some embodiments, the second expression control sequence comprises an MND promoter. In some embodiments, the expression vector further comprises an insulator having at least 95% sequence identity to a nucleic acid sequence selected from the group consisting of SEQ ID NO: 38, SEQ ID NO: 39, and SEQ ID NO: 40. In some embodiments, the expression vector further comprises an insulator having a nucleic acid sequence comprising any one of SEQ ID NOS: 38, 39, and 40. In some embodiments, the host cell is a hematopoietic stem cell.

In an eighth aspect of the present disclosure there is provided a pharmaceutical composition comprising an expression vector comprising a first expression control sequence operably linked to a first nucleic acid sequence, the first nucleic acid sequence encoding a shRNA to knockdown HPRT; and a second expression control sequence operably linked to a second nucleic acid sequence, the second nucleic acid sequence encoding a Wiskott-Aldrich Syndrome protein; and a pharmaceutically acceptable carrier.

In a ninth aspect of the present disclosure there is provided a pharmaceutical composition comprising a host cell transduced with an expression vector comprising a first expression control sequence operably linked to a first nucleic acid sequence, the first nucleic acid sequence encoding a shRNA to knockdown HPRT; and a second expression control sequence operably linked to a second nucleic acid sequence, the second nucleic acid sequence encoding a Wiskott-Aldrich Syndrome protein; and a pharmaceutically acceptable carrier.

In a tenth aspect of the present disclosure there is provided a method of selecting transduced cells comprising: transducing a population of cells with an expression vector comprising a first expression control sequence operably linked to a first nucleic acid sequence, the first nucleic acid sequence encoding a shRNA to knockdown HPRT; and a second expression control sequence operably linked to a second nucleic acid sequence, the second nucleic acid sequence encoding a Wiskott-Aldrich Syndrome protein; and enriching the population of transduced cells by selecting for the transduced cells with a purine analog. In some embodiments, the purine analog is selected from the group consisting of 6-thioguanine (“6TG”) and 6-mercaptopurin. In some embodiments, the transduced cells are HSCs. In some embodiments, the HSCs are allogenic HSCs. In some embodiments, the HSCs are autologous HSCs. In some embodiments, the HSCs are sibling matched HSCs.

In an eleventh aspect of the present disclosure there is provided a method of alleviating pathologies associated with Wiskott-Aldrich Syndrome comprising administering a therapeutically effective amount of transduced host cells to a patient in need of treatment thereof, wherein the transduced host cells are prepared by transducing a population of host cells with an expression vector comprising a first expression control sequence operably linked to a first nucleic acid sequence, the first nucleic acid sequence encoding a shRNA to knockdown HPRT; and a second expression control sequence operably linked to a second nucleic acid sequence, the second nucleic acid sequence encoding a Wiskott-Aldrich Syndrome protein. In some embodiments, the pathologies associated with Wiskott-Aldrich Syndrome are selected from the group consisting of microthrombocytopenia, eczema, autoimmune diseases, and recurrent infections. In some embodiments, the recurrent infections include recurrent cutaneous infections. In some embodiments, the recurrent infections are selected from the group consisting of otitis media, skin abscess, pneumonia, enterocolitis, meningitis, sepsis, and urinary tract infection. In some embodiments, the eczema is treatment-resistant eczema. In some embodiments, the autoimmune diseases are selected from the group consisting of hemolytic anemia, vasculitis, arthritis, neutropenia, inflammatory bowel disease, and IgA nephropathy, Henoch-Schönlein-like purpura, dermatomyositis, recurrent angioedema, and uveitis.

In a twelfth aspect of the present disclosure there is provided a polynucleotide comprising a first nucleic acid sequence having at least 95% sequence identity to that of SEQ ID NO: 14, and a second nucleic acid sequence having at least 95% sequence identity to that of SEQ ID NO: 15. In some embodiments, the polynucleotide further comprises a nucleic acid sequence having SEQ ID NO: 13. In some embodiments, the polynucleotide further comprises a nucleic acid sequence having SEQ ID NO: 41. In some embodiments, the polynucleotide further comprises a nucleic acid sequence having SEQ ID NO: 31. In some embodiments, the polynucleotide further comprises a nucleic acid sequence selected from the group consisting of SEQ ID NO: 38, SEQ ID NO: 39, and SEQ ID NO: 40.

In some embodiments, the first nucleic acid sequence is located upstream of the second nucleic acid sequence. In some embodiments, the first nucleic acid sequence has a same orientation as the second nucleic acid sequence. In some embodiments, the same orientation is a forward orientation. In some embodiments, the first nucleic acid sequence has a different orientation as the second nucleic acid sequence. In some embodiments, the different orientation is a reverse orientation.

In some embodiments, the first nucleic acid sequence is located downstream of the second nucleic acid sequence. In some embodiments, the first nucleic acid sequence has a same orientation as the second nucleic acid sequence. In some embodiments, the same orientation is a forward orientation. In some embodiments, the first nucleic acid sequence comprises a different orientation as the second nucleic acid sequence. In some embodiments, the different orientation is a reverse orientation.

In some embodiments, the first nucleic acid sequence is oriented in a first direction, wherein the second nucleic acid sequence is oriented in a second direction, and where the first and second directions are opposite. In some embodiments, the first nucleic acid sequence is located downstream from the second nucleic acid sequence. In some embodiments, the first nucleic acid sequence is located upstream from the second nucleic acid sequence.

In some embodiments, the first nucleic acid sequence is oriented in the same direction as the second nucleic acid sequence. In some embodiments, the first nucleic acid sequence is located downstream from the second nucleic acid sequence. In some embodiments, the first nucleic acid sequence is located upstream from the second nucleic acid sequence.

In a thirteenth aspect of the present disclosure there is provided a polynucleotide comprising a nucleic acid sequence having at least 90% identity to any one of SEQ ID NOS: 42-57. In some embodiments, the nucleic acid sequence has at least 95% identity to any one of SEQ ID NOS: 42-57. In some embodiments, the nucleic acid sequence has at least 96% identity to any one of SEQ ID NOS: 42-57. In some embodiments, the nucleic acid sequence has at least 97% identity to any one of SEQ ID NOS: 42-57. In some embodiments, the nucleic acid sequence has at least 98% identity to any one of SEQ ID NOS: 42-57. In some embodiments, the nucleic acid sequence has at least 99% identity to any one of SEQ ID NOS: 42-57.

In a fourteenth aspect of the present disclosure there is provided a polynucleotide having a nucleic acid sequence of any one of SEQ ID NOS: 42-57.

In a fifteenth aspect of the present disclosure there is provided an expression vector comprising (a) a nucleic acid sequence encoding pTL20c; (b) a nucleic acid encoding a WASP expression cassette; and (c) a nucleic acid encoding a 7sk/sh734 expression cassette. In some embodiments, the expression vector further comprises a nucleic acid sequence encoding an insulator. In some embodiments, the WASP expression cassette is located upstream of the 7sk/sh734 expression cassette. In some embodiments, the expression vector having the WASP expression cassette located upstream of the 7sk/sh734 expression cassette has a nucleic acid sequence having at least 90% identity to any of one SEQ ID NOS: 44, 45, 48, and 49. In some embodiments, the expression vector having the WASP expression cassette located upstream of the 7sk/sh734 expression cassette has a nucleic acid sequence having at least 95% identity to any of one SEQ ID NOS: 44, 45, 48, and 49. In some embodiments, the expression vector having the WASP expression cassette located upstream of the 7sk/sh734 expression cassette has a nucleic acid sequence having at least 90% identity to any of one SEQ ID NOS: 51, 53, 55, and 57. In some embodiments, the expression vector having the WASP expression cassette located upstream of the 7sk/sh734 expression cassette has a nucleic acid sequence having at least 95% identity to any of one SEQ ID NOS: 51, 53, 55, and 57.

In some embodiments, the WASP expression cassette is located downstream of the 7sk/sh734 expression cassette. In some embodiments, the expression vector having the WASP expression cassette located downstream of the 7sk/sh734 expression cassette has a nucleic acid sequence having at least 90% identity to any of one SEQ ID NOS: 42, 43, 46, and 47. In some embodiments, the expression vector having the WASP expression cassette located downstream of the 7sk/sh734 expression cassette has a nucleic acid sequence having at least 95% identity to any of one SEQ ID NOS: 42, 43, 46, and 47. In some embodiments, the expression vector having the WASP expression cassette located downstream of the 7sk/sh734 expression cassette has a nucleic acid sequence having at least 90% identity to any of one SEQ ID NOS: 50, 52, 54, and 56. In some embodiments, the expression vector having the WASP expression cassette located downstream of the 7sk/sh734 expression cassette has a nucleic acid sequence having at least 95% identity to any of one SEQ ID NOS: 50, 52, 54, and 56.

In some embodiments, the 7sk/sh734 expression cassette and the WASP expression cassette are oriented in the same direction. In some embodiments, the 7sk/sh734 expression cassette and the WASP expression cassette are oriented in opposing directions. In some embodiments, the 7sk/sh734 expression cassette is oriented in a forward direction relative to the WASP cassette. In some embodiments, the 7sk/sh734 expression cassette is oriented in a reverse direction relative to the WASP expression cassette.

In some embodiments, the expression vector having the WASP expression cassette oriented in the same direction relative to the 7sk/sh734 expression cassette has a nucleic acid sequence having at least 90% identity to any of one SEQ ID NOS: 42, 44, 46, and 48. In some embodiments, the expression vector having the WASP expression cassette oriented in the same direction relative to the 7sk/sh734 expression cassette has a nucleic acid sequence having at least 95% identity to any of one SEQ ID NOS: 42, 44, 46, and 48. In some embodiments, the expression vector having the WASP expression cassette oriented in the same direction relative to the 7sk/sh734 expression cassette has a nucleic acid sequence having at least 90% identity to any of one SEQ ID NOS: 50, 51, 54, and 55. In some embodiments, the expression vector having the WASP expression cassette oriented in the same direction relative to the 7sk/sh734 expression cassette has a nucleic acid sequence having at least 95% identity to any of one SEQ ID NOS: 50, 51, 54, and 55.

In some embodiments, the expression vector having the WASP expression cassette oriented in an opposing direction relative to the 7sk/sh734 expression cassette has a nucleic acid sequence having at least 90% identity to any of one SEQ ID NOS: 43, 45, 47, and 49. In some embodiments, the expression vector having the WASP expression cassette oriented in an opposing direction relative to the 7sk/sh734 expression cassette has a nucleic acid sequence having at least 95% identity to any of one SEQ ID NOS: 43, 45, 47, and 49. In some embodiments, the expression vector having the WASP expression cassette oriented in an opposing direction relative to the 7sk/sh734 expression cassette has a nucleic acid sequence having at least 90% identity to any of one SEQ ID NOS: 52, 53, 56, and 57. In some embodiments, the expression vector having the WASP expression cassette oriented in an opposing direction relative to the 7sk/sh734 expression cassette has a nucleic acid sequence having at least 95% identity to any of one SEQ ID NOS: 52, 53, 56, and 57.

In a sixteenth aspect of the present disclosure is a polynucleotide comprising a nucleic acid sequence having at least 90% identity to SEQ ID NO: 58. In some embodiments, the nucleic acid sequence has at least 95% identity to SEQ ID NO: 58. In some embodiments, the nucleic acid sequence has at least 96% identity to SEQ ID NO: 58. In some embodiments, the nucleic acid sequence has at least 97% identity to SEQ ID NO: 58. In some embodiments, the nucleic acid sequence has at least 98% identity to SEQ ID NO: 58. In some embodiments, the nucleic acid sequence has at least 99% identity to SEQ ID NO: 58.

In a seventeenth aspect of the present disclosure is a polynucleotide having SEQ ID NO: 58.

In an eighteenth aspect of the present disclosure is a polynucleotide comprising a nucleic acid sequence having at least 90% identity to SEQ ID NO: 59. In some embodiments, the nucleic acid sequence has at least 95% identity to SEQ ID NO: 59. In some embodiments, the nucleic acid sequence has at least 96% identity to SEQ ID NO: 59. In some embodiments, the nucleic acid sequence has at least 97% identity to SEQ ID NO: 59. In some embodiments, the nucleic acid sequence has at least 98% identity to SEQ ID NO: 59. In some embodiments, the nucleic acid sequence has at least 99% identity to SEQ ID NO: 59.

In a nineteenth aspect of the present disclosure is a polynucleotide having SEQ ID NO: 59.

In a twentieth aspect of the present disclosure is a polynucleotide comprising a nucleic acid sequence having at least 90% identity to any one of SEQ ID NOS: 63 and 65. In some embodiments, the nucleic acid sequence has at least 95% identity to any one of SEQ ID NOS: 63 and 65. In some embodiments, the nucleic acid sequence has at least 96% identity to any one of SEQ ID NOS: 63 and 65. In some embodiments, the nucleic acid sequence has at least 97% identity to any one of SEQ ID NOS: 63 and 65. In some embodiments, the nucleic acid sequence has at least 98% identity to any one of SEQ ID NOS: 63 and 65. In some embodiments, the nucleic acid sequence has at least 99% identity to any one of SEQ ID NOS: 63 and 65.

In a twenty-first aspect of the present disclosure is a polynucleotide having any one of SEQ ID NOS: 63 and 65.

In a twenty-second aspect of the present disclosure is a polynucleotide comprising a nucleic acid sequence having at least 90% identity to any one of SEQ ID NOS: 64 and 66. In some embodiments, the nucleic acid sequence has at least 95% identity to any one of SEQ ID NOS: 64 and 66. In some embodiments, the nucleic acid sequence has at least 96% identity to any one of SEQ ID NOS: 64 and 66. In some embodiments, the nucleic acid sequence has at least 97% identity to any one of SEQ ID NOS: 64 and 66. In some embodiments, the nucleic acid sequence has at least 98% identity to any one of SEQ ID NOS: 64 and 66. In some embodiments, the nucleic acid sequence has at least 99% identity to any one of SEQ ID NOS: 64 and 66.

In a twenty-third aspect of the present disclosure is a polynucleotide having any one of SEQ ID NOS: 64 and 66.

It is believed that with a strategy of combined conditioning and chemoselection (such as with a purine analog), efficient and high engraftment of HPRT-deficient, Wiskott-Aldrich Syndrome protein-containing hematopoietic stem cells can be achieved, and it is believed that such high engraftment may be accomplished with low overall toxicity. It is believed that the enhanced engraftment and chemoselection of the gene-modified HSCs, combined with lineage-specific expression of Wiskott-Aldrich Syndrome protein, may result in a sufficient frequency of cells expressing Wiskott-Aldrich Syndrome protein. As a safety measure, HPRT-deficient cells can be negatively selected, such as by introducing a dihydrofolate reductase inhibitor, such as methotrexate (MTX) or mycophenolic acid (MPA), to inhibit the enzyme dihydrofolate reductase (DHFR) in the purine de novo synthetic pathway, thus killing HPRT deficient cells. It is further believed that HPRT-deficient HSCs can be selected in vivo using a regimen of a purine analog (e.g. 6TG) to enhance engraftment. It is also believed that the expanded gene-modified HSCs can differentiate into erythrocytes expressing the Wiskott-Aldrich Syndrome protein transgene.

### BRIEF DESCRIPTION OF THE FIGURES

FIG. 1A sets forth a diagram of a vector including a nucleic acid sequence encoding a human WASP gene under the control of an MND promoter.

FIG. 1B sets forth a diagram of a vector including (i) a nucleic acid sequence encoding a human WASP gene under the control of an MND promoter, and (ii) a nucleic acid sequence encoding a shRNA designed to knockdown HPRT, where the 7sk/sh734 expression cassette is located downstream of the hWASP expression cassette, in accordance with some embodiments of the present disclosure. In some embodiments, the human WASP gene is a wild-type human WASP gene (e.g. SEQ ID NO: 67) or a variant thereof (e.g. one that includes one, two, three, or four silent mutations) (e.g. SEQ ID NO: 68). In some embodiments, the human WASP gene is codon optimized (e.g. SEQ ID NO: 69). In some embodiments, the vector comprises at least 90% sequence identity to SEQ ID NO: 44. In some embodiments, the vector comprises at least 90% sequence identity to SEQ ID NO: 48.

FIG. 1C sets forth a diagram of a vector including (i) a nucleic acid sequence encoding a human WASP gene under the control of an MND promoter, and (ii) a nucleic acid sequence encoding a shRNA designed to knockdown HPRT, where the 7sk/sh734 expression cassette is located downstream of the hWASP expression cassette, in accordance with some embodiments of the present disclosure. As compared with the vector illustrated in FIG. 1B, the 7sk/sh734 expression cassette is comparatively oriented in a reverse orientation. In some embodiments, the human WASP gene is a wild-type human WASP gene (e.g. SEQ ID NO: 67) or a variant thereof (e.g. one that includes one, two, three, or four silent mutations) (e.g. SEQ ID NO: 68). In some embodiments, the human WASP gene is codon optimized (e.g. SEQ ID NO: 69). In some embodiments, the vector comprises at least 90% sequence identity to SEQ ID NO: 45. In some embodiments, the vector comprises at least 90% sequence identity to SEQ ID NO: 49.

FIG. 1D sets forth a diagram of a vector including (i) a nucleic acid sequence encoding a human WASP gene under the control of an MND promoter, and (ii) a nucleic acid sequence encoding a shRNA designed to knockdown HPRT, where the 7sk/sh734 expression cassette is located upstream of the hWASp expression cassette, in accordance with some embodiments of the present disclosure. In some embodiments, the human WASP gene is a wild-type human WASP gene (e.g. SEQ ID NO: 67) or a variant thereof (e.g. one that includes one, two, three, or four silent mutations) (e.g. SEQ ID NO: 68). In some embodiments, the human WASP gene is codon optimized (e.g. SEQ ID NO: 69). In some embodiments, the vector comprises at least 90% sequence identity to SEQ ID NO: 42. In some embodiments, the vector comprises at least 90% sequence identity to SEQ ID NO: 46.

FIG. 1E sets forth a diagram of a vector including (i) a nucleic acid sequence encoding a human WASP gene under the control of an MND promoter, and (ii) a nucleic acid sequence encoding a shRNA designed to knockdown HPRT, where the 7sk/sh734 expression cassette is located upstream of the hWASp expression cassette, in accordance with some embodiments of the present disclosure. As compared with the vector illustrated in FIG. 1D, the 7sk/sh734 expression cassette is comparatively oriented in a reverse orientation. In some embodiments, the human WASP gene is a wild-type human WASP gene (e.g. SEQ ID NO: 67) or a variant thereof (e.g. one that includes one, two, three, or four silent mutations) (e.g. SEQ ID NO: 68). In some embodiments, the human WASP gene is codon optimized (e.g. SEQ ID NO: 69). In some embodiments, the vector comprises at least 90% sequence identity to SEQ ID NO: 43. In some embodiments, the vector comprises at least 90% sequence identity to SEQ ID NO: 47.

FIG. 1F sets forth a diagram of a vector including (i) a nucleic acid sequence encoding a human WASP gene under the control of an MND promoter, and (ii) a nucleic acid sequence encoding a shRNA designed to knockdown HPRT, where the 7sk/sh734 expression cassette is located downstream of the hWASP expression cassette, in accordance with some embodiments of the present disclosure. In some embodiments, the human WASP gene is a wild-type human WASP gene (e.g. SEQ ID NO: 67) or a variant thereof (e.g. one that includes one, two, three, or four silent mutations) (e.g. SEQ ID NO: 68). In some embodiments, the human WASP gene is codon optimized (e.g. SEQ ID NO: 69). In some embodiments, the vector comprises at least 90% sequence identity to SEQ ID NO: 51. In some embodiments, the vector comprises at least 90% sequence identity to SEQ ID NO: 55.

FIG. 1G sets forth a diagram of a vector including (i) a nucleic acid sequence encoding a human WASP gene under the control of an MND promoter, and (ii) a nucleic acid sequence encoding a shRNA designed to knockdown HPRT, where the 7sk/sh734 expression cassette is located downstream of the hWASP expression cassette, in accordance with some embodiments of the present disclosure. As compared with the vector illustrated in FIG. 1F, the 7sk/sh734 expression cassette is comparatively oriented in a reverse orientation. In some embodiments, the human WASP gene is a wild-type human WASP gene (e.g. SEQ ID NO: 67) or a variant thereof (e.g. one that includes one, two, three, or four silent mutations) (e.g. SEQ ID NO: 68). In some embodiments, the human WASP gene is codon optimized (e.g. SEQ ID NO: 69). In some embodiments, the vector comprises at least 90% sequence identity to SEQ ID NO: 53. In some embodiments, the vector comprises at least 90% sequence identity to SEQ ID NO: 57.

FIG. 1H sets forth a diagram of a vector including (i) a nucleic acid sequence encoding a human WASP gene under the control of an MND promoter, and (ii) a nucleic acid sequence encoding a shRNA designed to knockdown HPRT, where the 7sk/sh734 expression cassette is located upstream of the hWASp expression cassette, in accordance with some embodiments of the present disclosure. In some embodiments, the human WASP gene is a wild-type human WASP gene (e.g. SEQ ID NO: 67) or a variant thereof (e.g. one that includes one, two, three, or four silent mutations) (e.g. SEQ ID NO: 68). In some embodiments, the human WASP gene is codon optimized (e.g. SEQ ID NO: 69). In some embodiments, the vector comprises at least 90% sequence identity to SEQ ID NO: 50. In some embodiments, the vector comprises at least 90% sequence identity to SEQ ID NO: 54.

FIG. 1I sets forth a diagram of a vector including (i) a nucleic acid sequence encoding a human WASP gene under the control of an MND promoter, and (ii) a nucleic acid sequence encoding a shRNA designed to knockdown HPRT, where the 7sk/sh734 expression cassette is located upstream of the hWASp expression cassette, in accordance with some embodiments of the present disclosure. As compared with the vector illustrated in FIG. 1H, the 7sk/sh734 expression cassette is comparatively oriented in a reverse orientation. In some embodiments, the human WASP gene is a wild-type human WASP gene (e.g. SEQ ID NO: 67) or a variant thereof (e.g. one that includes one, two, three, or four silent mutations) (e.g. SEQ ID NO: 68). In some embodiments, the human WASP gene is codon optimized (e.g. SEQ ID NO: 69). In some embodiments, the vector comprises at least 90% sequence identity to SEQ ID NO: 52. In some embodiments, the vector comprises at least 90% sequence identity to SEQ ID NO: 56.

FIG. 2 illustrates the secondary structure and theoretical primary DICER cleavage sites (arrows) of sh734 (see also SEQ ID NO: 26). The secondary structure has an MFE value of about −30.9 kcal/mol.

FIG. 3 illustrates the secondary RNA structure and minimum free energy (dG) for sh616 (see also SEQ ID NO: 23).

FIG. 4 illustrates the secondary RNA structure and minimum free energy (dG) for sh212 (see also SEQ ID NO: 24).

FIG. 5 illustrates a modified version of sh734 (sh734.1) (see also SEQ ID NO: 25). The secondary structure has an 1VIFE value of −36.16 kcal/mol.

FIG. 6 illustrates the de novo design of an artificial miRNA734 (111 nt) (see also SEQ ID NO: 19).

FIG. 7 illustrates the de novo design of an artificial miRNA211 (111 nt) (see also SEQ ID NO: 20).

FIG. 8 illustrates a sh734 embedded in the miRNA-3G backbone, a third generation miRNA scaffold derived from the native miRNA 16-2 structure (see also SEQ ID NO: 22).

FIG. 9 illustrates the sh211 embedded in the miRNA-3G backbone, a 3rd generation miRNA scaffold derived from the native miRNA 16-2 structure (see also SEQ ID NO: 21).

FIG. 10 illustrate human 7sk promoter mutations. Mutations (arrows) and deletions introduced into the cis-distal sequence enhancer (DSE) and proximal sequence enhancer (PSE) elements (long, wide boxes) in the 7sk promoter relative to the TATA box (tall, thin boxes) are illustrated. The mutations are also described by Boyd, D. C., Turner, P. C., Watkins, N.J., Gerster, T. & Murphy, S. Functional Redundancy of Promoter Elements Ensures Efficient Transcription of the Human 7SK Gene in vivo. Journal of Molecular Biology 253, 677-690 (1995), the disclosure of which is hereby incorporated by reference herein in its entirety.

FIG. 11 sets forth a flowchart illustrating methods of treating a subject with transduced HSCs, including the steps of conditioning and chemoselection in accordance with certain embodiments of the present disclosure.

FIG. 12 illustrates a 3 kb fragment containing the MND promoter, WASP codon-optimized cDNA, an WPRE element and 7SK/ShRNA. The fragment can be first “built” in any cloning plasmid vector allowing quick and easy cloning of different modules in different combinations. The expression cassette can then be isolated by MluI and NotI digestion and cloned into the unique MluI and NotI sites of a pT20Lc lentivirus vector to generate the final expression vector.

FIG. 13 illustrates a Moloney murine leukemia (MoMuLV) long terminal repeat. An MND promoter may be derived from the MoMuLV LTR.

FIG. 14 illustrates the relative expression of levels of HPRT and further illustrates a cutoff at which point HPRT deficient cells may be selected for with a purine analog.

FIG. 15 sets forth representative results of WASp+ cells and WASp expression for the pTL20c-MND/hWASwt-r7SK/sh734 and pTL20c-r7SK/sh734-MND/hWAS^{co} vector candidates (see Table 15).

FIG. 16 provides a graph of the percentage of WASp+ expression for the eight vector candidates set forth in Table 15 and for a control.

FIG. 17 provides a graph of the mean fluorescence intensity for the eight vector candidates set forth in Table 15 and for a control.

FIG. 18 provides a graph of the percentage of WASp+ Cells versus Vector Copy Number (VCN) for four different vector candidates.

FIG. 19 provides bar graphs illustrating the variation of WASp expression in MFI (Mean Fluorescence Intensity) per Vector Copy Number (VCN) for four different vector candidates.

FIG. 20A provides a graph showing an initial titration of 6TG for Jurkat cells. The optimal 6TG dose is further illustrated.

FIG. 20B provides a graph illustrating the vector copy number (VCN) after chemoselection of transduced Jurkat cells.

FIG. 21 schematically illustrates the irradiation of WASp knockout mice and the subsequent engraftment of murine HSCs.

FIG. 22A sets forth a vector map of pTL20c_SK734fwd_MND_WAS_650 (SEQ ID NO: 50).

FIG. 22B sets forth a vector map of pTL20c_MND_WAS_SK734fwd_650 (SEQ ID NO: 51).

FIG. 22C sets forth a vector map of pTL20c_SK734rev_MND_WAS_650(SEQ ID NO: 52).

FIG. 22D sets forth a vector map of pTL20c_MND_WAS_SK734rev_650 (SEQ ID NO: 53).

FIG. 22E sets forth a vector map of pTL20c_SK734fwd_MND_coWAS_650 (SEQ ID NO: 54).

FIG. 22F sets forth a vector map of pTL20c_MND_coWAS_SK734fwd_650 (SEQ ID NO: 55).

FIG. 22G sets forth a vector map of pTL20c_SK734rev_MND_coWAS_650 (SEQ ID NO: 56).

FIG. 22H sets forth a vector map of pTL20c_MND_coWAS_SK734rev 650 (SEQ ID NO: 57).

FIG. 23A provides a graph showing the relative titer levels measured following transduction of 293T cells with a lentiviral vector candidate.

FIG. 23B provides a graph showing the relative titer levels measured following transduction of 293T cells with a lentiviral vector candidate.

FIG. 24 provides a graph showing the relative titer levels measured following transduction of 293T cells with a lentiviral vector candidate.

### SEQUENCE LISTING

The nucleic acid and amino acid sequences appended hereto are shown using standard letter abbreviations for nucleotide bases, and three letter code for amino acids, as defined in 37 C.F.R. 1.822. The sequence listing is submitted as an ASCII text file, named “Calimmune-071WO_ST25.txt” created on Dec. 18, 2019, 311 KB, which is incorporated by reference herein.

### DETAILED DESCRIPTION

### Definitions

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless the context clearly indicates otherwise. Similarly, the word “or” is intended to include “and” unless the context clearly indicates otherwise.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the terms “comprising,” “including,” “having,” and the like are used interchangeably and have the same meaning. Similarly, “comprises,” “includes,” “has,” and the like are used interchangeably and have the same meaning. Specifically, each of the terms is to be interpreted as an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

As used herein, the terms “administer” or “administering” refer to providing a composition, formulation, or specific agent to a subject (e.g. a human patient) in need of treatment, including those described herein.

As used herein, the term “expression cassette” refers to one or more genetic sequences within a vector which can express a RNA, and, in some embodiments, subsequently a protein. The expression cassette comprises at least one promoter and at least one gene of interest. In some embodiments, the expression cassette includes at least one promoter, at least one gene of interest, and at least one additional nucleic acid sequence encoding a molecule for expression (e.g. a RNAi). In some embodiments, expression cassette is positionally and sequentially oriented within the vector such that the nucleic acid in the cassette can be transcribed into RNA, and when necessary, translated into a protein or a polypeptide, undergo appropriate post-translational modifications required for activity in the transformed cell (e.g. transduced stem cell), and be translocated to the appropriate compartment for biological activity by targeting to appropriate intracellular compartments or secretion into extracellular compartments. In some embodiments, the cassette has its 3′ and 5′ ends adapted for ready insertion into a vector, e.g., it has restriction endonuclease sites at each end.

As used herein, the term “functional nucleic acid” refers to molecules having the capacity to reduce expression of a protein by directly interacting with a transcript that encodes the protein. siRNA molecules, ribozymes, and antisense nucleic acids constitute exemplary functional nucleic acids.

As used herein, the term “gene” refers broadly to any segment of DNA associated with a biological function. A gene encompasses sequences including but not limited to a coding sequence, a promoter region, a cis-regulatory sequence, a non-expressed DNA segment is a specific recognition sequence for regulatory proteins, a non-expressed DNA segment that contributes to gene expression, a DNA segment designed to have desired parameters, or combinations thereof.

As used herein, the term “gene silencing” is meant to describe the downregulation, knock-down, degradation, inhibition, suppression, repression, prevention, or decreased expression of a gene, transcript and/or polypeptide product. Gene silencing and interference also describe the prevention of translation of mRNA transcripts into a polypeptide. In some embodiments, translation is prevented, inhibited, or decreased by degrading mRNA transcripts or blocking mRNA translation.

As used herein, the term “gene expression” refers to the cellular processes by which a biologically active polypeptide is produced from a DNA sequence.

As used herein, the terms “hematopoietic cell transplant” or “hematopoietic cell transplantation” refer to bone marrow transplantation, peripheral blood stem cell transplantation, umbilical vein blood transplantation, or any other source of pluripotent hematopoietic stem cells. Likewise, the terms “stem cell transplant,” or “transplant,” refer to a composition comprising stem cells that are in contact with (e.g. suspended in) a pharmaceutically acceptable carrier. Such compositions are capable of being administered to a subject through a catheter.

As used herein, the term “host cell” refers to cells that is to be modified using the methods of the present disclosure. In some embodiments, the host cells are mammalian cells in which the expression vector can be expressed. Suitable mammalian host cells include, but are not limited to, human cells, murine cells, non-human primate cells (e.g. rhesus monkey cells), human progenitor cells or stem cells, 293 cells, HeLa cells, D17 cells, MDCK cells, BHK cells, and Cf2Th cells. In certain embodiments, the host cell comprising an expression vector of the disclosure is a hematopoietic cell, such as hematopoietic progenitor/stem cell (e.g. CD34-positive hematopoietic progenitor/stem cell), a monocyte, a macrophage, a peripheral blood mononuclear cell, a CD4+T lymphocyte, a CD8+ T lymphocyte, or a dendritic cell. The hematopoietic cells (e.g. CD4+T lymphocytes, CD8+ T lymphocytes, and/or monocyte/macrophages) to be transduced with an expression vector of the disclosure can be allogeneic, autologous, or from a matched sibling. The hematopoietic cells are, in some embodiments, CD34-positive and can be isolated from the patient's bone marrow or peripheral blood. The isolated CD34-positive hematopoietic cells (and/or other hematopoietic cell described herein) is, in some embodiments, transduced with an expression vector as described herein.

As used herein, the term “hematopoietic stem cells” or “HSCs” refer to multipotent cells capable of differentiating into all the cell types of the hematopoietic system, including, but not limited to, granulocytes, monocytes, erythrocytes, megakaryocytes, lymphocytes, dendritic cells; and self-renewal activity, i.e. the ability to divide and generate at least one daughter cell with the identical (e.g., self-renewing) characteristics of the parent cell.

As used herein, “HPRT” is an enzyme involved in purine metabolism encoded by the HPRT1 gene. HPRT1 is located on the X chromosome, and thus is present in single copy in males. HPRT1 encodes the transferase that catalyzes the conversion of hypoxanthine to inosine monophosphate and guanine to guanosine monophosphate by transferring the 5-phosphorobosyl group from 5-phosphoribosyl 1-pyrophosphate to the purine. The enzyme functions primarily to salvage purines from degraded DNA for use in renewed purine synthesis (see also FIG. 37).

As used herein, the term “lentivirus” refers to a genus of retroviruses that are capable of infecting dividing and non-dividing cells. Several examples of lentiviruses include HIV (human immunodeficiency virus: including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (Hy), which causes immune deficiency in cats; bovine immune deficiency virus (BIV), which causes lymphadenopathy, lymphocytosis, and possibly central nervous system infection in cattle; and simian immunodeficiency virus (SIV), which causes immune deficiency and encephalopathy in sub-human primates.

As used herein, the term “lentiviral vector” is used to denote any form of a nucleic acid derived from a lentivirus and used to transfer genetic material into a cell via transduction. The term encompasses lentiviral vector nucleic acids, such as DNA and RNA, encapsulated forms of these nucleic acids, and viral particles in which the viral vector nucleic acids have been packaged.

As used herein, the terms “knock down” or “knockdown” when used in reference to an effect of RNAi on gene expression, means that the level of gene expression is inhibited, or is reduced to a level below that generally observed when examined under substantially the same conditions, but in the absence of RNAi.

As used herein, the term “minicell” refers to anucleate forms of bacterial cells, engendered by a disturbance in the coordination, during binary fission, of cell division with DNA segregation. Minicells are distinct from other small vesicles that are generated and released spontaneously in certain situations and, in contrast to minicells, are not due to specific genetic rearrangements or episomal gene expression. Minicells of the present disclosure are anucleate forms of E. coli or other bacterial cells, engendered by a disturbance in the coordination, during binary fission, of cell division with DNA segregation. Prokaryotic chromosomal replication is linked to normal binary fission, which involves mid-cell septum formation. In E. coli, for example, mutation of min genes, such as minCD, can remove the inhibition of septum formation at the cell poles during cell division, resulting in production of a normal daughter cell and an anucleate minicell. See de Boer et al., 1992; Raskin & de Boer, 1999; Hu & Lutkenhaus, 1999; Harry, 2001. Minicells are distinct from other small vesicles that are generated and released spontaneously in certain situations and, in contrast to minicells, are not due to specific genetic rearrangements or episomal gene expression. For practicing the present disclosure, it is desirable for minicells to have intact cell walls (“intact minicells”). In addition to min operon mutations, anucleate minicells also are generated following a range of other genetic rearrangements or mutations that affect septum formation, for example in the divIVB1 in B. subtilis. See Reeve and Cornett, 1975; Levin et al., 1992. Minicells also can be formed following a perturbation in the levels of gene expression of proteins involved in cell division/chromosome segregation. For example, overexpression of minE leads to polar division and production of minicells. Similarly, chromosome-less minicells may result from defects in chromosome segregation for example the smc mutation in Bacillus subtilis (Britton et al., 1998), spoOJ deletion in B. subtilis (Ireton et al., 1994), mukB mutation in E. coli (Hiraga et al., 1989), and parC mutation in E. coli (Stewart and D'Ari, 1992). Gene products may be supplied in trans. When over-expressed from a high-copy number plasmid, for example, CafA may enhance the rate of cell division and/or inhibit chromosome partitioning after replication (Okada et al., 1994), resulting in formation of chained cells and anucleate minicells (Wachi et al., 1989; Okada et al., 1993). Minicells can be prepared from any bacterial cell of Gram-positive or Gram-negative origin.

As used herein, the terms “miRNA” or “microRNA” refer to small non-coding RNAs of 20-22 nucleotides, typically excised from an about 70 nucleotide foldback RNA precursor structures known as pre-miRNAs. miRNAs negatively regulate their targets in one of two ways depending on the degree of complementarity between the miRNA and the target. First, miRNAs that bind with perfect or nearly perfect complementarity to protein-coding mRNA sequences induce the RNA-mediated interference (RNAi) pathway. miRNAs that exert their regulatory effects by binding to imperfect complementary sites within the 3′ untranslated regions (UTRs) of their mRNA targets, repress target-gene expression post-transcriptionally, apparently at the level of translation, through a RISC complex that is similar to, or possibly identical with, the one that is used for the RNAi pathway. Consistent with translational control, miRNAs that use this mechanism reduce the protein levels of their target genes, but the mRNA levels of these genes are only minimally affected. miRNAs encompass both naturally occurring miRNAs as well as artificially designed miRNAs that can specifically target any mRNA sequence. For example, in one embodiment, the skilled artisan can design short hairpin RNA constructs expressed as human miRNA (e.g., miR-30 or miR-21) primary transcripts. This design adds a Drosha processing site to the hairpin construct and has been shown to greatly increase knockdown efficiency (Pusch et al., 2004). The hairpin stem consists of 22-nt of dsRNA (e.g., antisense has perfect complementarity to desired target) and an about 15 to about 19 nucleotide loop from a human miR. It is believed that by adding the miR loop and miR30 flanking sequences on either or both sides of the hairpin results in greater than 10-fold increase in Drosha and DICER processing of the expressed hairpins when compared with conventional shRNA designs without microRNA. Increased Drosha and DICER processing translates into greater siRNA/miRNA production and greater potency for expressed hairpins.

As used herein, the term “mutated” refers to a change in a sequence, such as a nucleotide or amino acid sequence, from a native, standard, or reference version of the respective sequence, i.e. the non-mutated sequence. A mutated gene can result in a mutated gene product. A mutated gene product will differ from the non-mutated gene product by one or more amino acid residues. In some embodiments, a mutated gene which results in a mutated gene product can have a sequence identity of about 70%, about 75%, about 80%, about 85%, about 90%, about 95%, or greater to the corresponding non-mutated nucleotide sequence.

As used herein, the term “nanocapsules” refers to nanoparticles having a shell, e.g. a polymeric shell, encapsulating one or more components, e.g. one or more proteins and/or one or more nucleic acids. In some embodiments, the nanocapsules have an average diameter of less than or equal to about 200 nanometers (nm), for example between about 1 to 200 nm, or between about 5 to about 200 nm, or between about 10 to about 150 nm, or 15 to 100 nm, or between about 15 to about 150 nm, or between about 20 to about 125 nm, or between about 50 to about 100 nm, or between about 50 to about 75 nm. In other embodiments, the nanocapsules have an average diameter of between about 10 nm to about 20 nm, about 20 to about 25 nm, about 25 nm to about 30 nm, about 30 nm to about 35 nm, about 35 nm to about 40 nm, about 40 nm to about 45 nm, about 45 nm to about 50 nm, about 50 nm to about 55 nm, about 55 nm to about 60 nm, about 60 nm to about 65 nm, about 70 to about 75 nm, about 75 nm to about 80 nm, about 80 nm to about 85 nm, about 85 nm to about 90 nm, about 90 nm to about 95 nm, about 95 nm to about 100 nm, or about 100 nm to about 110 nm. In some embodiments, the nanocapsules are designed to degrade in about 1 hour, or about 2 hours, or about 3 hours, or about 4 hours, or about 5 hours, or about 6 or about 12 hours, or about 1 day, or about 2 days, or about 1 week, or about 1 month. In some embodiments, the surface of the nanocapsule can have a charge between about 1 to about 15 millivolts (mV) (such as measured in a standard phosphate solution). In other embodiments, the surface of the nanocapsule can have a charge of between about 1 to about 10 mV.

As used herein, the term “operably linked” refers to functional linkage between a nucleic acid expression control sequence (such as a promoter, signal sequence, enhancer or array of transcription factor binding sites) and a second nucleic acid sequence, wherein the expression control sequence affects transcription and/or translation of the nucleic acid corresponding to the second sequence when the appropriate molecules (e.g., transcriptional activator proteins) are bound to the expression control sequence.

As used herein, the term “promoter” refers to a recognition site of a polynucleotide (DNA or RNA) to which an RNA polymerase binds. An RNA polymerase initiates and transcribes polynucleotides operably linked to the promoter. In some embodiments, promoters operative in mammalian cells comprise an AT-rich region located approximately 25 to 30 bases upstream from the site where transcription is initiated and/or another sequence found about 70 to about 80 bases upstream from the start of transcription, e.g. a CNCAAT region where N may be any nucleotide.

As used herein, the term “retroviruses” refers to viruses having an RNA genome that is reverse transcribed by retroviral reverse transcriptase to a cDNA copy that is integrated into the host cell genome. Retroviral vectors and methods of making retroviral vectors are known in the art. Briefly, to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., Cell, Vol. 33:153-159, 1983). When a recombinant plasmid containing a cDNA, together with the retroviral LTR and packaging sequences, is introduced into this cell line, the packaging sequence allows the RNA transcript of the recombinant plasmid to be packaged into viral particles, which are then secreted into the culture media. The media containing the recombinant retroviruses is then collected, optionally concentrated, and used for gene transfer.

As used herein, the terms “siRNA” or “small interference RNA” refer to a short double-strand RNA composed of about ten nucleotides to several tens of nucleotides, which induce RNAi (RNA interference), i.e. induce the degradation of the target mRNA or inhibit the expression of the target gene via cleavage of the target mRNA. RNA interference (“RNAi”) is a method of post-transcriptional inhibition of gene expression that is conserved throughout many eukaryotic organisms, and it refers to a phenomenon in which a double-stranded RNA composed of a sense RNA having a sequence homologous to the mRNA of the target gene and an antisense RNA having a sequence complementary thereto is introduced into cells or the like so that it can selectively induce the degradation of the mRNA of the target gene or can inhibit the expression of the target gene. RNAi is induced by a short (i.e., less than about 30 nucleotides) double-stranded RNA molecule present in cells (Fire A. et al., Nature, 391: 806-811, 1998). When siRNA is introduced into cells, the expression of the mRNA of the target gene having a nucleotide sequence complementary to that of the siRNA will be inhibited.

As used herein, the terms “small hairpin RNA” or “shRNA” refer to RNA molecules comprising an antisense region, a loop portion and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme DICER, which is a member of the RNase III family. As used herein, the phrase “post-transcriptional processing” refers to mRNA processing that occurs after transcription and is mediated, for example, by the enzymes DICER and/or Drosha.

As used herein, the term “subject” refers to a mammal such as a human, mouse or primate. Typically, the mammal is a human (Homo sapiens).

As used herein, the term “therapeutic gene” refers to a gene that can be administered to a subject for the purpose of treating or preventing a disease.

As used herein, the terms “transduce” or “transduction” refer to the delivery of a gene(s) using a viral or retroviral vector by means of infection rather than by transfection. For example, an anti-HPRT gene carried by a retroviral vector (a modified retrovirus used as a vector for introduction of nucleic acid into cells) can be transduced into a cell through infection and provirus integration. Thus, a “transduced gene” is a gene that has been introduced into the cell via lentiviral or vector infection and provirus integration. Viral vectors (e.g., “transducing vectors”) transduce genes into “target cells” or host cells.

As used herein, the terms “treatment,” “treating,” or “treat,” with respect to a specific condition, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment”, as used herein, covers any treatment of a disease or disorder in a subject, particularly in a human, and includes: (a) preventing the disease or disorder from occurring in a subject which may be predisposed to the disease but has not yet been diagnosed as having it; (b) inhibiting the disease or disorder, i.e., arresting its development; and (c) relieving or alleviating the disease or disorder, i.e., causing regression of the disease or disorder and/or relieving one or more disease or disorder symptoms. “Treatment” can also encompass delivery of an agent or administration of a therapy in order to provide for a pharmacologic effect, even in the absence of a disease, disorder or condition. The term “treatment” is used in some embodiments to refer to administration of a compound of the present disclosure to mitigate a disease or a disorder in a host, preferably in a mammalian subject, more preferably in humans. Thus, the term “treatment” can include includes: preventing a disorder from occurring in a host, particularly when the host is predisposed to acquiring the disease but has not yet been diagnosed with the disease; inhibiting the disorder; and/or alleviating or reversing the disorder. As far as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure can occur prior to onset of a disease. The term does not mean that the disease state must be completely avoided.

As used herein, the term “vector” refers to a nucleic acid molecule capable of mediating entry of, e.g., transferring, transporting, etc., another nucleic acid molecule into a cell. The transferred nucleic acid is generally linked to, e.g., inserted into, the vector nucleic acid molecule. A vector may include sequences that direct autonomous replication or may include sequences sufficient to allow integration into host cell DNA. As will be evident to one of ordinary skill in the art, viral vectors may include various viral components in addition to nucleic acid(s) that mediate entry of the transferred nucleic acid. Numerous vectors are known in the art including, but not limited to, linear polynucleotides, polynucleotides associated with ionic or amphiphilic compounds, plasmids, and viral vectors. Examples of viral vectors include, but are not limited to, adenoviral vectors, adeno-associated virus vectors, retroviral vectors (including lentiviral vectors), and the like.

Expression Vectors

The present disclosure provides, in some embodiments, expression vectors (e.g. lentiviral expression vectors) including at least two nucleic acid sequences for expression. In some embodiments, the nucleic acid sequences encode a nucleic acid molecule (e.g. RNA, mRNA, or another molecule which may be found in the cytoplasm of a cell). In some embodiments, the expression vectors include a first nucleic acid sequence encoding an agent designed to knockdown the HPRT gene or otherwise effectuate a decrease in HPRT expression. In some embodiments, the expression vectors include a second nucleic acid encoding a Wiskott-Aldrich Syndrome protein.

In some embodiments, the expression vector is a self-inactivating lentiviral vector. In other embodiments, the expression vector is a retroviral vector. A lentiviral genome is generally organized into a 5′ long terminal repeat (LTR), the gag gene, the pol gene, the env gene, the accessory genes (nef, vif, vpr, vpu) and a 3′ LTR. The viral LTR is divided into three regions called U3, R and U5. The U3 region contains the enhancer and promoter elements. The U5 region contains the polyadenylation signals. The R (repeat) region separates the U3 and U5 regions and transcribed sequences of the R region appear at both the 5′ and 3′ ends of the viral RNA. See, for example, “RNA Viruses: A Practical Approach” (Alan J. Cann, Ed., Oxford University Press, (2000)); O Narayan and Clements (1989) J. Gen. Virology, Vol. 70:1617-1639; Fields et al. (1990) Fundamental Virology Raven Press.; Miyoshi H, Blamer U, Takahashi M, Gage F H, Verma I M. (1998) J Virol., Vol. 72(10):8150 7, and U.S. Pat. No. 6,013,516. Examples of lentiviral vectors that have been used to infect HSCs are described in the publications which follows, each of which are hereby incorporated herein by reference in their entireties: Evans et al., Hum Gene Ther., Vol. 10:1479-1489, 1999; Case et al., Proc Natl Acad Sci USA, Vol. 96:2988-2993, 1999; Uchida et al., Proc Natl Acad Sci USA, Vol. 95:11939-11944, 1998; Miyoshi et al., Science, Vol. 283:682-686, 1999; and Sutton et al., J. Virol., Vol. 72:5781-5788, 1998.

In some embodiments, the expression vector is a modified lentivirus, and thus is able to infect both dividing and non-dividing cells. In some embodiments, the modified lentiviral genome lacks genes for lentiviral proteins required for viral replication, thus preventing undesired replication, such as replication in the target cells. In some embodiments, the required proteins for replication of the modified genome are provided in trans in the packaging cell line during production of the recombinant retrovirus or lentivirus.

In some embodiments, the expression vector comprises sequences from the 5′ and 3′ long terminal repeats (LTRs) of a lentivirus. In some embodiments, the vector comprises the R and U5 sequences from the 5′ LTR of a lentivirus and an inactivated or self-inactivating 3′ LTR from a lentivirus. In some embodiments, the LTR sequences are HIV LTR sequences.

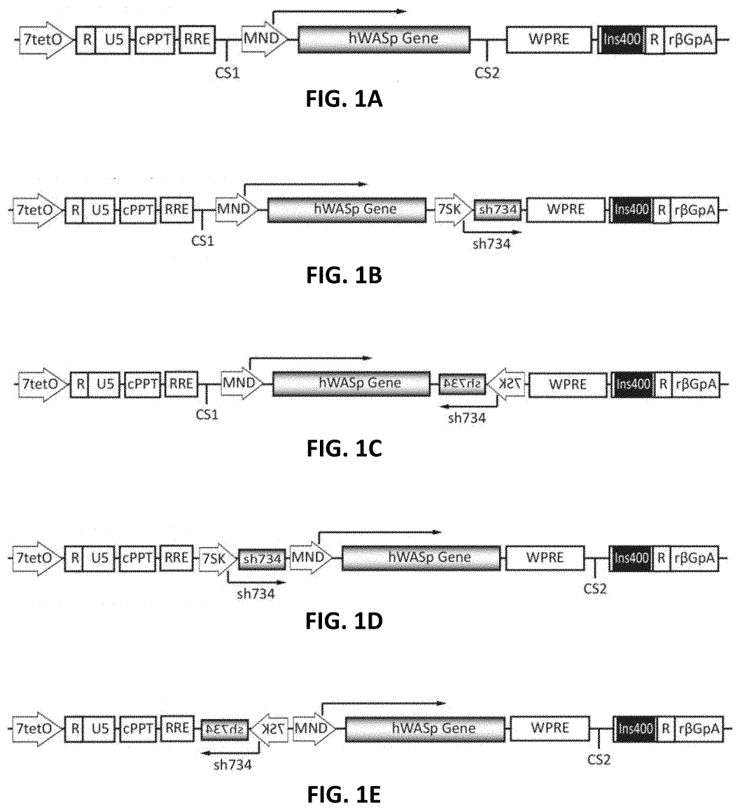

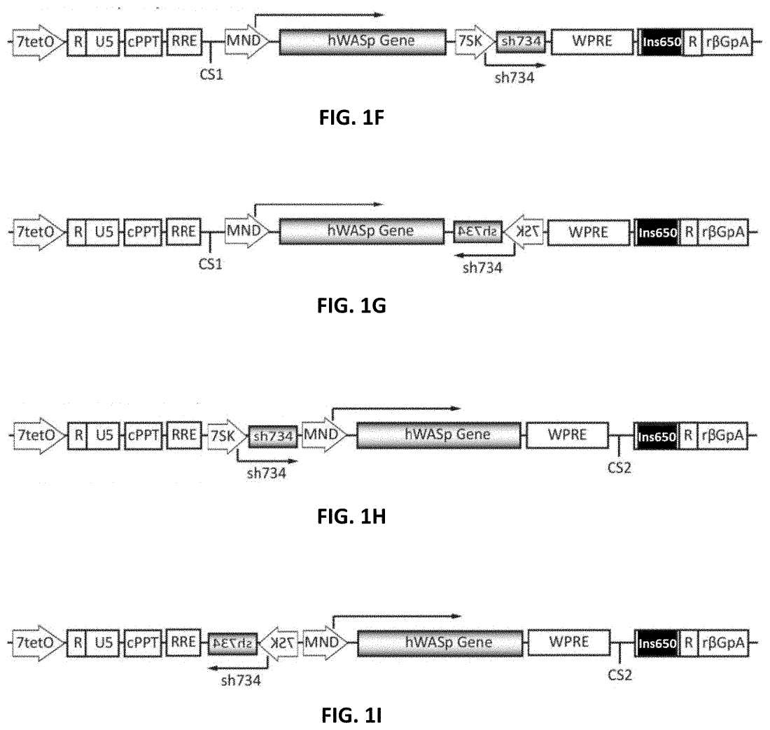

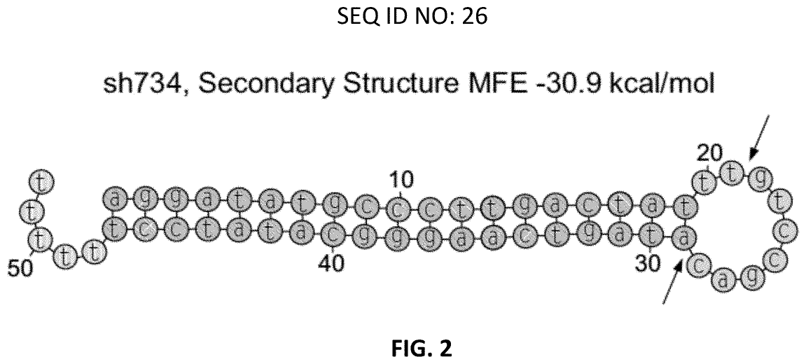

Additional components of a lentiviral expression vector (and methods of synthesizing and/or producing such vectors) are disclosed in United States Patent Application Publication No. 2018/0112220, the disclosure of which is hereby incorporated by reference herein in its entirety. For example, the lentiviral expression vectors may include one or more of a central polypurine tract (e.g. having SEQ ID NO: 41), a WPRE element (e.g. having SEQ ID NO: 13), and a Rev response element (e.g. having SEQ ID NO: 38). These additional elements are illustrated, for example, in FIGS. 1A to 1E.

In some embodiments, the lentiviral vectors contemplated herein may be integrative or non-integrating (also referred to as an integration defective lentivirus). As used herein, the term “integration defective lentivirus” or “IDLV” refers to a lentivirus having an integrase that lacks the capacity to integrate the viral genome into the genome of the host cells. In some applications, the use of by an integrating lentivirus vector may avoid potential insertional mutagenesis induced by an integrating lentivirus. Integration defective lentiviral vectors typically are generated by mutating the lentiviral integrase gene or by modifying the attachment sequences of the LTRs (see, e.g., Sarkis et al., Curr. Gene. Ther., 6: 430-437 (2008)). Lentiviral integrase is coded for by the HIV-1 Pol region and the region cannot be deleted as it encodes other critical activities including reverse transcription, nuclear import, and viral particle assembly. Mutations in pol that alter the integrase protein fall into one of two classes: those which selectively affect only integrase activity (Class I); or those that have pleiotropic effects (Class II). Mutations throughout the N and C terminals and the catalytic core region of the integrase protein generate Class II mutations that affect multiple functions including particle formation and reverse transcription. Class I mutations limit their affect to the catalytic activities, DNA binding, linear episome processing and multimerization of integrase. The most common Class I mutation sites are a triad of residues at the catalytic core of integrase, including D64, D116, and E152. Each mutation has been shown to efficiently inhibit integration with a frequency of integration up to four logs below that of normal integrating vectors while maintaining transgene expression of the NILV. Another alternative method for inhibiting integration is to introduce mutations in the integrase DNA attachment site (LTR att sites) within a 12 base-pair region of the U3 region or within an 11 base-pair region of the U5 region at the terminal ends of the 5′ and 3′ LTRs, respectively. These sequences include the conserved terminal CA dinucleotide which is exposed following integrase-mediated end-processing. Single or double mutations at the conserved CA/TG dinucleotide result in up to a three to four log reduction in integration frequency; however, it retains all other necessary functions for efficient viral transduction.

In some embodiments the vector is an adeno-associated virus (AAV) vector. As used herein, the term “adeno-associated virus (AAV) vector” means an AAV viral particle containing an AAV vector genome (which, in turn, comprises the first and second expression cassettes referred to herein). It is meant to include AAV vectors of all serotypes, preferably AAV-1 through AAV-9, more preferably AAV-1, AAV-2, AAV-4, AAV-5, AAV-6, AAV-7, AAV-8, AAV-9, and combinations thereof. AAV vectors resulting from the combination of different serotypes may be referred to as hybrid AAV vectors. In one embodiment, the AAV vector is selected from the group consisting of AAV-1, AAV-2, AAV-4, AAV-5 and AAV-6, and combinations thereof. In one embodiment, the AAV vector is an AAV-5 vector. In one embodiment, the AAV vector is an AAV-5 vector comprising AAV-2 inverted terminal repeats (ITRs). Also contemplated by the present disclosure are AAV vectors comprising variants of the naturally occurring viral proteins, e.g., one or more capsid proteins.

Components to Effectuate the Knockdown of the HPRT Gene

In some embodiments, the nucleic acid sequence encoding the agent designed to knockdown the HPRT gene or otherwise effectuate a decrease in its expression is an RNA interference agent (RNAi). In some embodiments, the RNAi agent is an shRNA, a microRNA, or a hybrid thereof.

RNAi

In some embodiments, the expression vector comprises a first nucleic acid sequence encoding an RNAi. RNA interference is an approach for post-transcriptional silencing of gene expression by triggering degradation of homologous transcripts through a complex multistep enzymatic process, e.g. a process involving sequence-specific double-stranded small interfering RNA (siRNA). A simplified model for the RNAi pathway is based on two steps, each involving a ribonuclease enzyme. In the first step, the trigger RNA (either dsRNA or miRNA primary transcript) is processed into a short, interfering RNA (siRNA) by the RNase II enzymes DICER and Drosha. In the second step, siRNAs are loaded into the effector complex RNA-induced silencing complex (RISC). The siRNA is unwound during RISC assembly and the single-stranded RNA hybridizes with mRNA target. It is believed that gene silencing is a result of nucleolytic degradation of the targeted mRNA by the RNase H enzyme Argonaute (Slicer). If the siRNA/mRNA duplex contains mismatches the mRNA is not cleaved. Rather, gene silencing is a result of translational inhibition.

In some embodiments, the RNAi agent is an inhibitory or silencing nucleic acid. As used herein, a “silencing nucleic acid” refers to any polynucleotide which is capable of interacting with a specific sequence to inhibit gene expression. Examples of silencing nucleic acids include RNA duplexes (e.g. siRNA, shRNA), locked nucleic acids (“LNAs”), antisense RNA, DNA polynucleotides which encode sense and/or antisense sequences of the siRNA or shRNA, DNAzymses, or ribozymes. The skilled artisan will appreciate that the inhibition of gene expression need not necessarily be gene expression from a specific enumerated sequence, and may be, for example, gene expression from a sequence controlled by that specific sequence.

Methods for constructing interfering RNAs are known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA may be assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering RNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. When expressed, such an RNA molecule desirably forms a “hairpin” structure and is referred to herein as an “shRNA.” In some embodiments, the loop region is generally between about 2 and about 10 nucleotides in length (by way of example only, see SEQ ID NO: 32). In other embodiments, the loop region is from about 6 to about 9 nucleotides in length. In some embodiments, the sense region and the antisense region are between about 15 and about 30 nucleotides in length. Following post-transcriptional processing, the small hairpin RNA is converted into a siRNA by a cleavage event mediated by the enzyme DICER, which is a member of the RNase III family. The siRNA is then capable of inhibiting the expression of a gene with which it shares homology. Further details are described by see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); and Yu et al. Proc NatlAcadSci USA 99:6047-6052, (2002), the disclosures of which are hereby incorporated by reference herein in their entireties.

shRNA

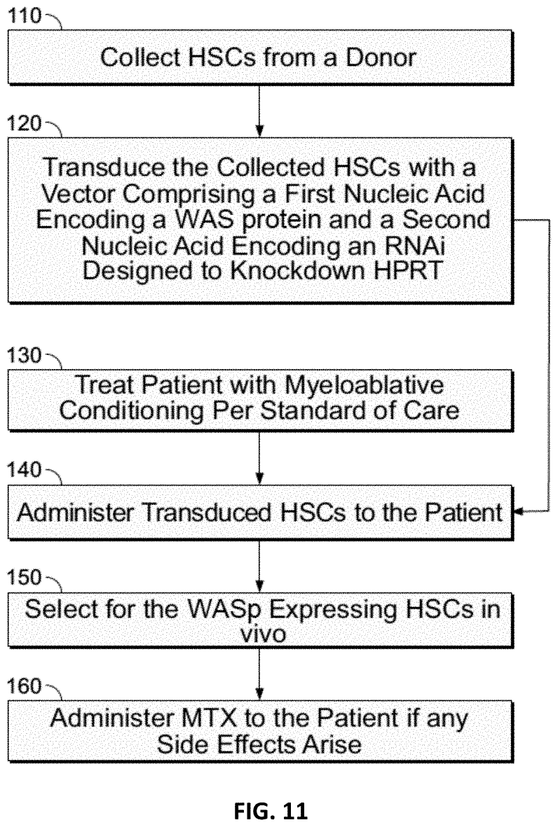

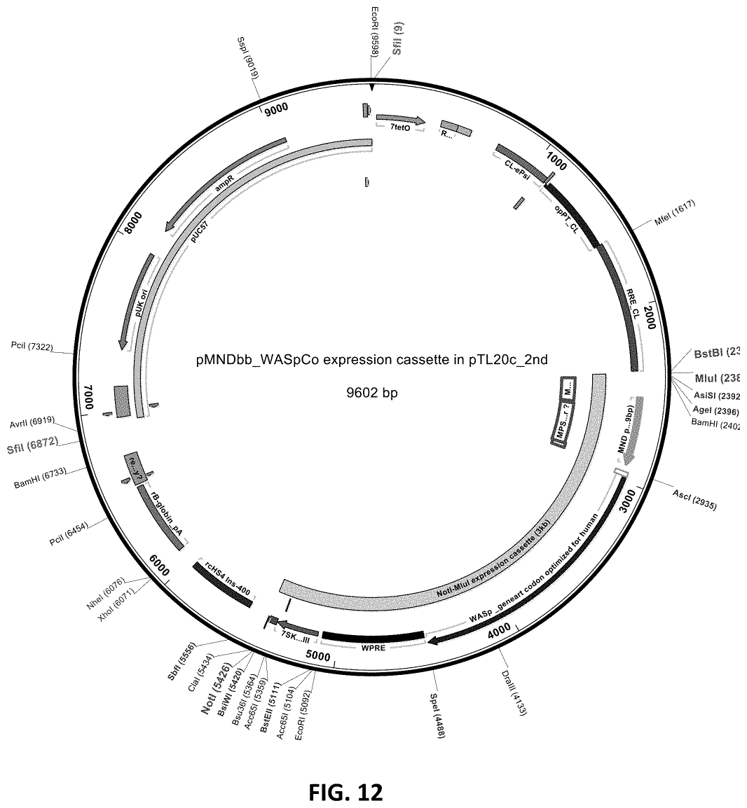

In some embodiments, the first nucleic acid sequence encodes a shRNA targeting an HPRT gene. In some embodiments, the first nucleic acid sequence encoding a shRNA targeting an HPRT gene has a sequence having at least 80% identity to that of SEQ ID NO: 26 (referred to herein as “sh734”). In some embodiments, the first nucleic acid sequence encoding a shRNA targeting an HPRT gene has a sequence having at least 85% identity to that of SEQ ID NO: 26. In other embodiments, the first nucleic acid sequence encoding a shRNA targeting an HPRT gene has a sequence having at least 90% identity to that of SEQ ID NO: 26. In yet other embodiments the first nucleic acid sequence encoding a shRNA targeting an HPRT gene has a sequence having at least 95% identity to that of SEQ ID NO: 26. In yet other embodiments the first nucleic acid sequence encoding a shRNA targeting an HPRT gene has a sequence having at least 96% identity to that of SEQ ID NO: 26. In further embodiments, the first nucleic acid sequence encoding a shRNA targeting an HPRT gene has a sequence having at least 97% identity to that of SEQ ID NO: 26. In even further embodiments, the first nucleic acid sequence encoding a shRNA targeting an HPRT gene has a sequence having at least 98% identity to that of SEQ ID NO: 26. In yet further embodiments, the first nucleic acid sequence encoding a shRNA targeting an HPRT gene has a sequence having at least 99% identity to that of SEQ ID NO: 26. In other embodiments, the first nucleic acid sequence encoding a shRNA targeting an HPRT gene has the sequence of SEQ ID NO: 26 (see also FIG. 11).

In some embodiments, the nucleic acid sequence of SEQ ID NO: 26 may be modified. In some embodiments, modifications include: (i) the incorporation of a hsa-miR-22 loop sequence (e.g. CCUGACCCA) (SEQ ID NO: 33); (ii) the addition of a 5′-3′ nucleotide spacer, such as one having two or three nucleotides (e.g. TA); (iii) a 5′ start modification, such as the addition of one or more nucleotides (e.g. G); and/or (iv) the addition of two nucleotides 5′ and 3′ to the stem and loop (e.g. 5′ A and 3′ T). In general, first generation shRNAs are processed into a heterogenous mix of small RNAs, and the accumulation of precursor transcripts has been shown to induce both sequence-dependent and independent nonspecific off-target effects in vivo. Therefore, based on the current understanding of DICER processing and specificity, design rules were applied design that would optimize the structure of the sh734 and DICER processivity and efficiency (see also Gu, S., Y. Zhang, L. Jin, Y. Huang, F. Zhang, M. C. Bassik, M. Kampmann, and M. A. Kay. 2014. Weak base pairing in both seed and 3′ regions reduce RNAi off-targets and enhances si/shRNA designs. Nucleic Acids Research 42:12169-12176).

In some embodiments, the nucleic acid sequence of SEQ ID NO: 26 is modified by adding two nucleotides 5′ and 3′ (e.g., G and C, respectively) to the hairpin loop (SEQ ID NO: 32), thereby lengthening the guide strand from about 19 nucleotides to about 21 nucleotides in length and replacing the loop with the hsa-miR-22 loop CCUGACCCA (SEQ ID NO: 33), to provide the nucleotide sequence of SEQ ID NO: 27. In some embodiments, the nucleic acid sequence encoding a shRNA targeting an HPRT gene has a sequence having at least 90% identity to that of SEQ ID NO: 27. In other embodiments, the first nucleic acid sequence encoding a shRNA targeting an HPRT gene has a sequence having at least 95% identity to that of SEQ ID NO: 27. In other embodiments, the first nucleic acid sequence encoding a shRNA targeting an HPRT gene has a sequence having at least 96% identity to that of SEQ ID NO: 27. In other embodiments, the first nucleic acid sequence encoding a shRNA targeting an HPRT gene has a sequence having at least 97% identity to that of SEQ ID NO: 27. In other embodiments, the first nucleic acid sequence encoding a shRNA targeting an HPRT gene has a sequence having at least 98% identity to that of SEQ ID NO: 27. In other embodiments, the first nucleic acid sequence encoding a shRNA targeting an HPRT gene has a sequence having at least 99% identity to that of SEQ ID NO: 27. In yet other embodiments, the nucleic acid sequence encoding a shRNA targeting an HPRT gene has the sequence of SEQ ID NO: 27. It is believed that the shRNA encoded by SEQ ID NO: 27 achieves similar knockdown of HPRT as compared with SEQ ID NO: 26. Likewise, it is believed that a cell rendered HPRT deficient through the knockdown of HPRT via expression of the shRNA encoded by SEQ ID NO: 27 allows for selection using a thioguanine analog (e.g. 6TG).

In some embodiments, the RNAi present within the vector encodes for a nucleic acid molecule, such as one having at least 95% sequence identity to any one of SEQ ID NO: 16 or SEQ ID NO: 17. In other embodiments, the RNAi present within the vector encodes for a nucleic acid molecule, such as one having at least 97% sequence identity to any one of SEQ ID NO: 16 or SEQ ID NO: 17. In some embodiments, the RNAi present within the vector encodes for a nucleic acid molecule, such as one having SEQ ID NO: 16 or SEQ ID NO: 17. In some embodiments, the nucleic acid molecules having SEQ ID NO: 16 or SEQ ID NO: 17 are found in the cytoplasm of a host cell. In some embodiments, the present disclosure provides for a host cell including at least one nucleic acid molecule selected from SEQ ID NO: 16 or SEQ ID NO: 17.

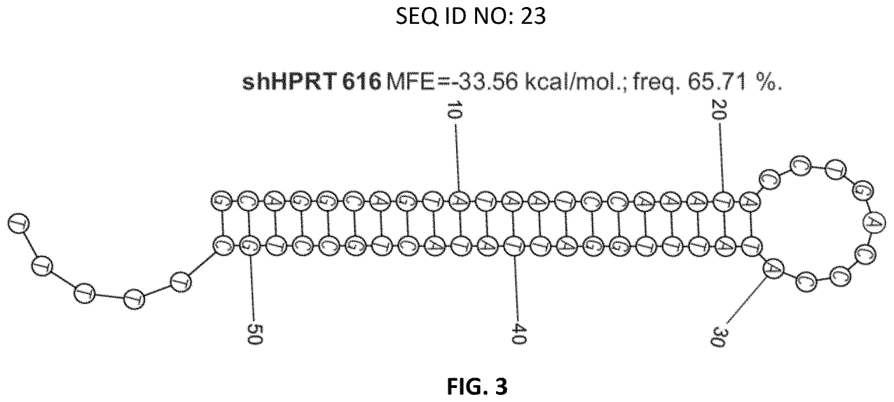

In some embodiments, the first nucleic acid sequence encoding a shRNA targeting an HPRT gene has a sequence having at least 80% identity to that of SEQ ID NO: 23 (referred to herein as “shHPRT 616”). In some embodiments, the first nucleic acid sequence encoding a shRNA targeting an HPRT gene has a sequence having at least 85% identity to that of SEQ ID NO: 23. In other embodiments, the nucleic acid sequence encoding a shRNA targeting an HPRT gene has a sequence having at least 90% identity to that of SEQ ID NO: 23. In yet other embodiments, the nucleic acid sequence encoding a shRNA targeting an HPRT gene shRNA has a sequence having at least 95% identity to that of SEQ ID NO: 23. In yet other embodiments, the nucleic acid sequence encoding a shRNA targeting an HPRT gene shRNA has a sequence having at least 96% identity to that of SEQ ID NO: 23. In further embodiments, the nucleic acid sequence encoding a shRNA targeting an HPRT gene has a sequence having at least 97% identity to that of SEQ ID NO: 23. In even further embodiments, the nucleic acid sequence encoding a shRNA targeting an HPRT gene has a sequence having at least 98% identity to that of SEQ ID NO: 23. In yet further embodiments, the nucleic acid sequence encoding a shRNA targeting an HPRT gene has a sequence having at least 99% identity to that of SEQ ID NO: 23. In other embodiments, the nucleic acid sequence encoding a shRNA targeting an HPRT gene has the sequence of SEQ ID NO: 23 (see also FIG. 3).

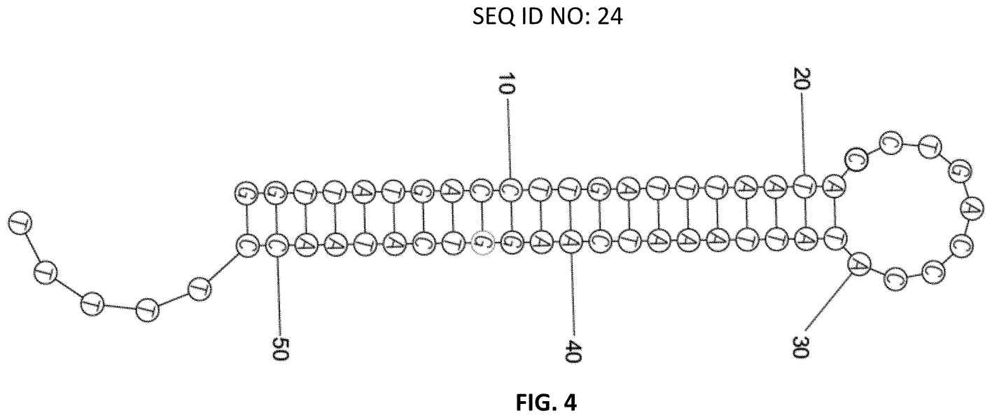

In some embodiments, the first nucleic acid sequence encoding a shRNA targeting an HPRT gene has a sequence having at least 80% identity to that of SEQ ID NO: 24 (referred to herein as “shHPRT 211”). In some embodiments, the first nucleic acid sequence encoding a shRNA targeting an HPRT gene has a sequence having at least 85% identity to that of SEQ ID NO: 24. In other embodiments, the nucleic acid sequence encoding a shRNA targeting an HPRT gene has a sequence having at least 90% identity to that of SEQ ID NO: 24. In yet other embodiments, the nucleic acid sequence encoding a shRNA targeting an HPRT gene shRNA has a sequence having at least 95% identity to that of SEQ ID NO: 24. In yet other embodiments, the nucleic acid sequence encoding a shRNA targeting an HPRT gene shRNA has a sequence having at least 96% identity to that of SEQ ID NO: 24. In further embodiments, the nucleic acid sequence encoding a shRNA targeting an HPRT gene has a sequence having at least 97% identity to that of SEQ ID NO: 24. In even further embodiments, the nucleic acid sequence encoding a shRNA targeting an HPRT gene has a sequence having at least 98% identity to that of SEQ ID NO: 24. In yet further embodiments, the nucleic acid sequence encoding a shRNA targeting an HPRT gene has a sequence having at least 99% identity to that of SEQ ID NO: 24. In other embodiments, the nucleic acid sequence encoding a shRNA targeting an HPRT gene has the sequence of SEQ ID NO: 24 (see also FIG. 4).

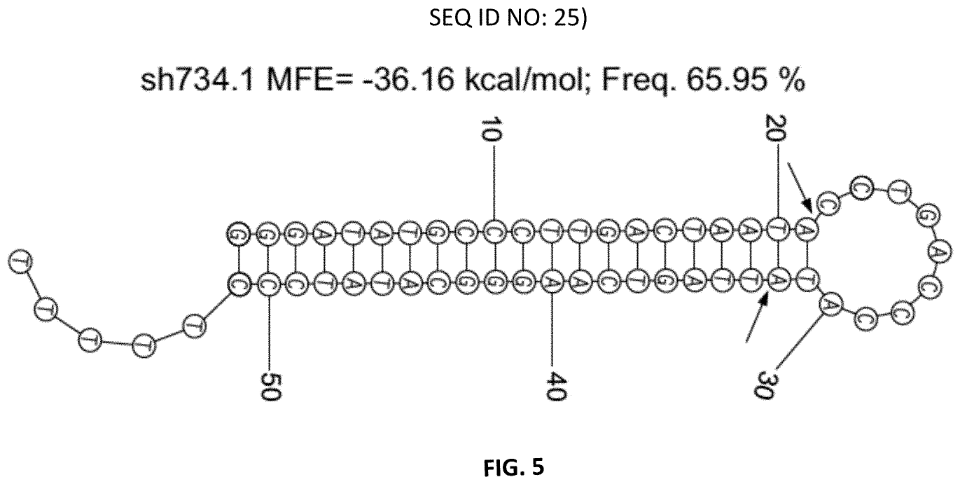

In some embodiments, the nucleic acid sequence encoding a shRNA targeting an HPRT gene has a sequence having at least 80% identity to that of SEQ ID NO: 25 (referred to herein as “shHPRT 734.1”). In some embodiments, the nucleic acid sequence encoding a shRNA targeting an HPRT gene has a sequence having at least 85% identity to that of SEQ ID NO: 25. In other embodiments, the nucleic acid sequence encoding a shRNA targeting an HPRT gene has a sequence having at least 90% identity to that of SEQ ID NO: 25. In yet other embodiments, the nucleic acid sequence encoding a shRNA targeting an HPRT gene shRNA has a sequence having at least 95% identity to that of SEQ ID NO: 25. In yet other embodiments, the nucleic acid sequence encoding a shRNA targeting an HPRT gene shRNA has a sequence having at least 96% identity to that of SEQ ID NO: 25. In further embodiments, the nucleic acid sequence encoding a shRNA targeting an HPRT gene has a sequence having at least 97% identity to that of SEQ ID NO: 25. In even further embodiments, the nucleic acid sequence encoding a shRNA targeting an HPRT gene has a sequence having at least 98% identity to that of SEQ ID NO: 25. In yet further embodiments, the nucleic acid sequence encoding a shRNA targeting an HPRT gene has a sequence having at least 99% identity to that of SEQ ID NO: 25. In other embodiments, the nucleic acid sequence encoding a shRNA targeting an HPRT gene has the sequence of SEQ ID NO: 25 (see also FIG. 5).

MicroRNAs

MicroRNAs (miRs) are a group of non-coding RNAs which post-transcriptionally regulate the expression of their target genes. It is believed that these single stranded molecules form a miRNA-mediated silencing complex (miRISC) complex with other proteins which bind to the 3′ untranslated region (UTR) of their target mRNAs so as to prevent their translation in the cytoplasm.

In some embodiments, shRNA sequences are embedded into micro-RNA secondary structures (“micro-RNA based shRNA”). In some embodiments, shRNA nucleic acid sequences targeting HPRT are embedded within micro-RNA secondary structures. In some embodiments, the micro-RNA based shRNAs target coding sequences within HPRT to achieve knockdown of HPRT expression, which is believed to be equivalent to the utilization of shRNA targeting HPRT without attendant pathway saturation and cellular toxicity or off-target effects. In some embodiments, the micro-RNA based shRNA is a de novo artificial microRNA shRNA. The production of such de novo micro-RNA based shRNAs are described by Fang, W. & Bartel, David P. The Menu of Features that Define Primary MicroRNAs and Enable De Novo Design of MicroRNA Genes. Molecular Cell 60, 131-145, the disclosure of which is hereby incorporated by reference herein in its entirety.

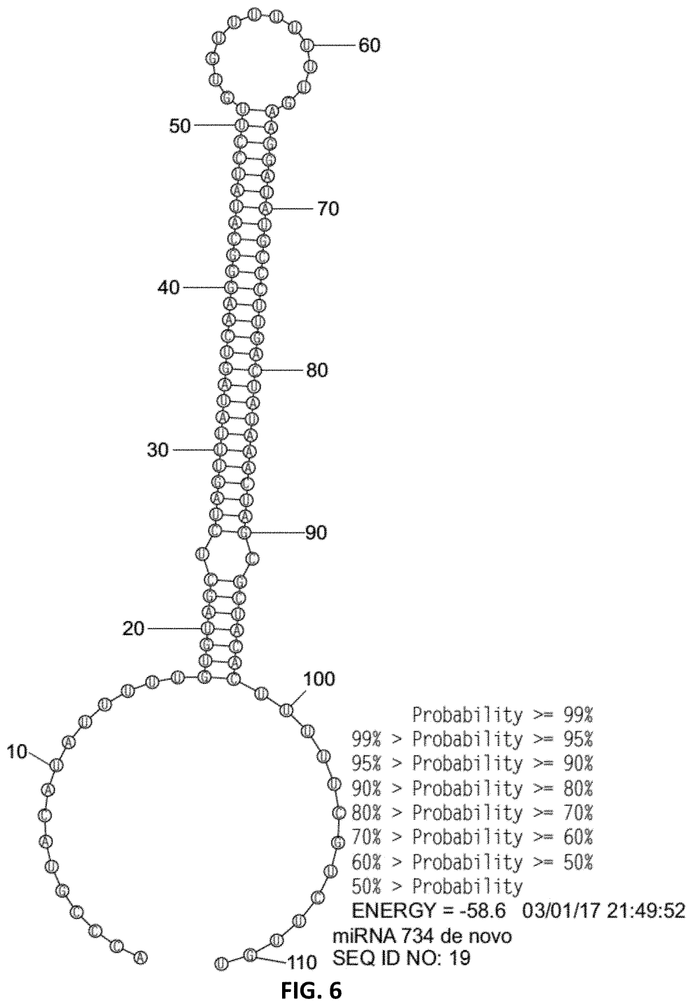

In some embodiments, the micro-RNA based shRNA has a nucleic acid sequence having at least 80% identity to that of SEQ ID NO: 34. In some embodiments, the micro-RNA based shRNA has a nucleic acid sequence having at least 85% identity to that of SEQ ID NO: 34. In some embodiments, the micro-RNA based shRNA has nucleic acid a sequence having at least 90% identity to that of SEQ ID NO: 34. In some embodiments, the micro-RNA based shRNA has nucleic acid a sequence having at least 95% identity to that of SEQ ID NO: 34. In some embodiments, the micro-RNA based shRNA has nucleic acid a sequence having at least 96% identity to that of SEQ ID NO: 34. In some embodiments, the micro-RNA based shRNA has nucleic acid a sequence having at least 97% identity to that of SEQ ID NO: 34. In some embodiments, the micro-RNA based shRNA has nucleic acid a sequence having at least 98% identity to that of SEQ ID NO: 34. In some embodiments, the micro-RNA based shRNA has nucleic acid a sequence having at least 98% identity to that of SEQ ID NO: 34. In some embodiments, the micro-RNA based shRNA has the nucleic acid sequence of SEQ ID NO: 34 (“miRNA734-Denovo”) (see also FIG. 6). The RNA form of SEQ ID NO: 34 is found at SEQ ID NO: 19.

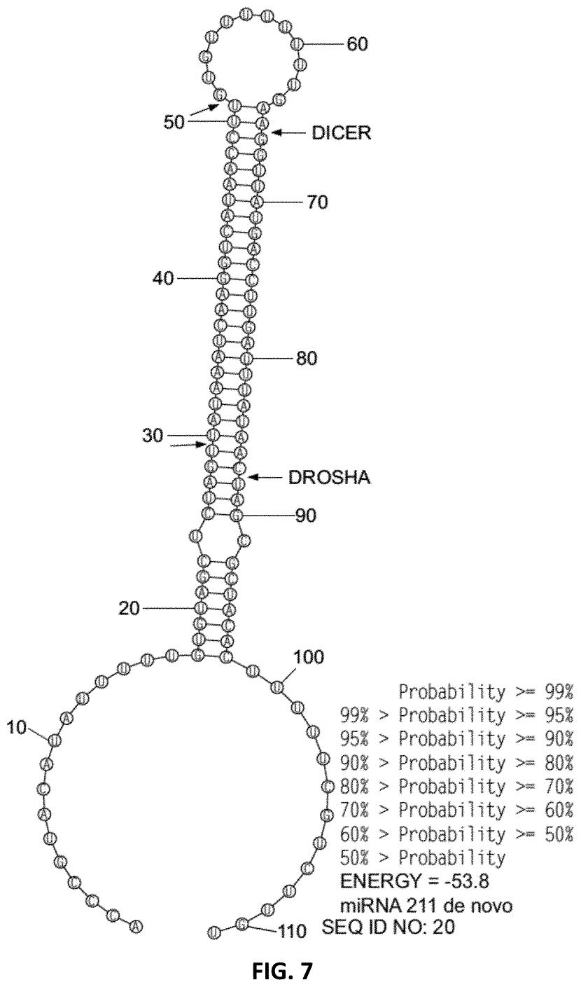

In some embodiments, the micro-RNA based shRNA has nucleic acid a sequence having at least 80% identity to that of SEQ ID NO: 35. In some embodiments, the micro-RNA based shRNA has nucleic acid a sequence having at least 85% identity to that of SEQ ID NO: 35. In some embodiments, the micro-RNA based shRNA has nucleic acid a sequence having at least 90% identity to that of SEQ ID NO: 35. In some embodiments, the micro-RNA based shRNA has nucleic acid a sequence having at least 95% identity to that of SEQ ID NO: 35. In some embodiments, the micro-RNA based shRNA has nucleic acid a sequence having at least 96% identity to that of SEQ ID NO: 35. In some embodiments, the micro-RNA based shRNA has nucleic acid a sequence having at least 97% identity to that of SEQ ID NO: 35. In some embodiments, the micro-RNA based shRNA has nucleic acid a sequence having at least 98% identity to that of SEQ ID NO: 35. In some embodiments, the micro-RNA based shRNA has nucleic acid a sequence having at least 99% identity to that of SEQ ID NO: 35. In some embodiments, the micro-RNA based shRNA has the nucleic acid sequence of SEQ ID NO: 35 (“miRNA211-Denovo”) (see also FIG. 7). The RNA form of SEQ ID NO: 35 is found at SEQ ID NO: 20.

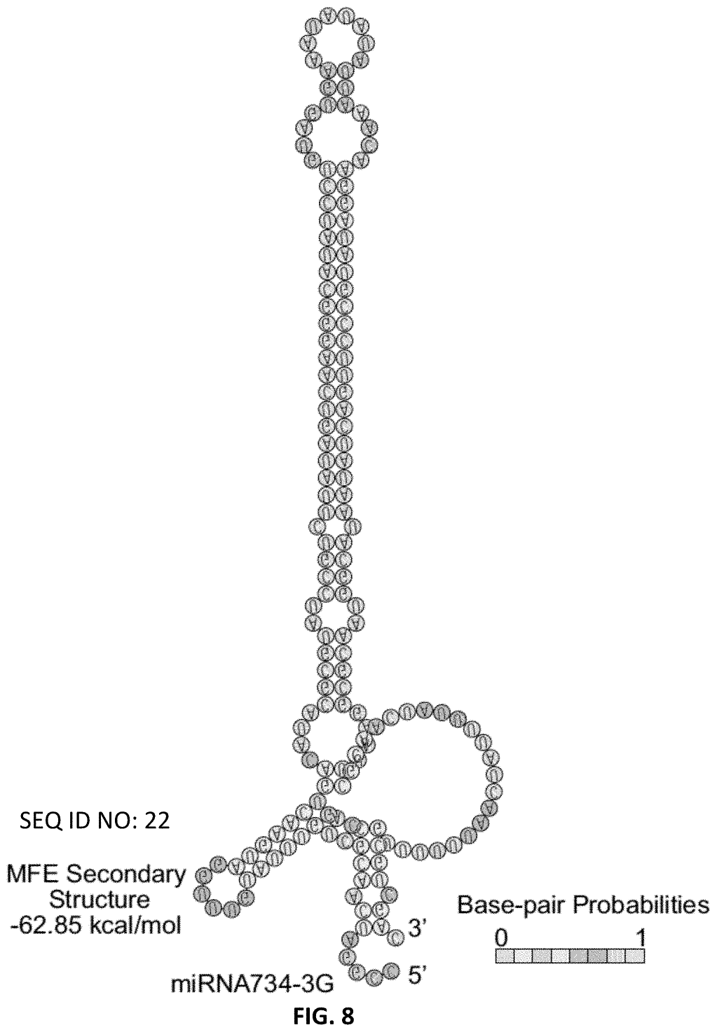

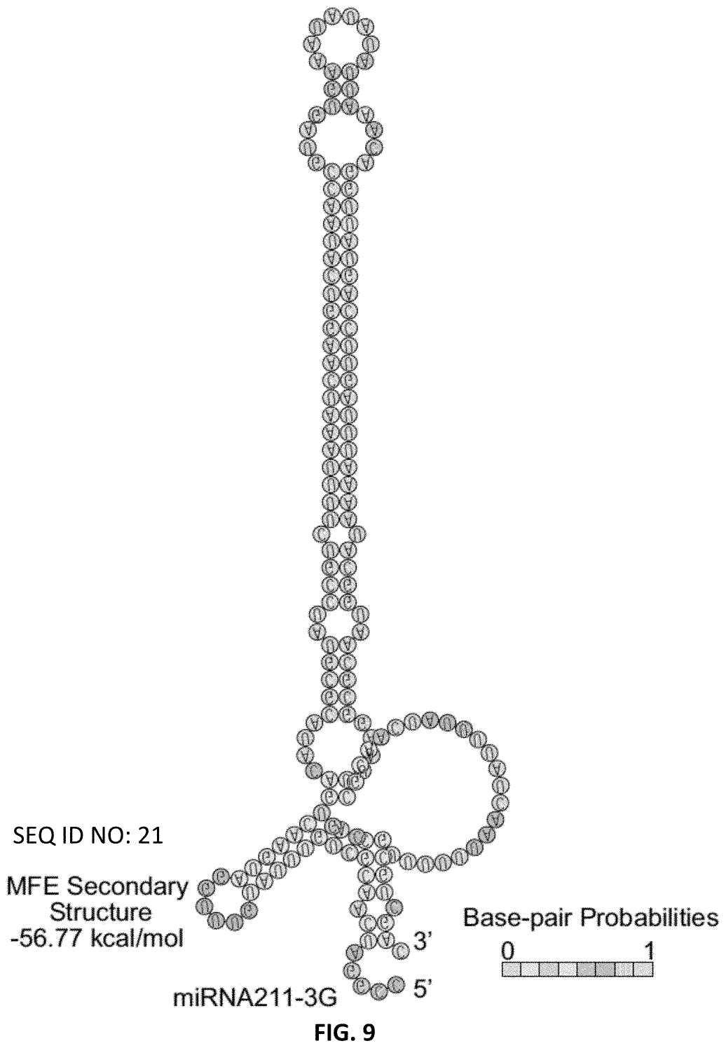

In other embodiments, the micro-RNA based shRNA is a third generation miRNA scaffold modified miRNA 16-2 (hereinafter “miRNA-3G”) (see, e.g., FIGS. 8 and 9). The synthesis of such miRNA-3G molecules is described by Watanabe, C., Cuellar, T. L. & Haley, B. “Quantitative evaluation of first, second, and third generation hairpin systems reveals the limit of mammalian vector-based RNAi,” RNA Biology 13, 25-33 (2016), the disclosure of which is hereby incorporated by reference herein in its entirety.

In some embodiments, the miRNA-3G has a nucleic acid sequence having at least 80% identity to that of SEQ ID NO: 21. In some embodiments, the miRNA-3G has a nucleic acid sequence having at least 85% identity to that of SEQ ID NO: 21. In some embodiments, the miRNA-3G has a nucleic acid sequence having at least 90% identity to that of SEQ ID NO: 21. In some embodiments, the micro-RNA based shRNA has a nucleic acid sequence having at least 95% identity to that of SEQ ID NO: 35. In some embodiments, the micro-RNA based shRNA has a nucleic acid sequence having at least 96% identity to that of SEQ ID NO: 35. In some embodiments, the micro-RNA based shRNA has a nucleic acid sequence having at least 97% identity to that of SEQ ID NO: 35. In some embodiments, the micro-RNA based shRNA has a nucleic acid sequence having at least 98% identity to that of SEQ ID NO: 35. In some embodiments, the micro-RNA based shRNA has a nucleic acid sequence having at least 99% identity to that of SEQ ID NO: 35. In some embodiments, the miRNA-3G has the nucleic acid sequence of SEQ ID NO: 21 (“miRNA211-3G”) (see also FIG. 9).

In some embodiments, the miRNA-3G has a nucleic acid sequence having at least 80% identity to that of SEQ ID NO: 22. In some embodiments, the miRNA-3G has a nucleic acid sequence having at least 85% identity to that of SEQ ID NO: 22. In some embodiments, the miRNA-3G has a nucleic acid sequence having at least 90% identity to that of SEQ ID NO: 22. In some embodiments, the miRNA-3G has a nucleic acid sequence having at least 95% identity to that of SEQ ID NO: 22. In some embodiments, the miRNA-3G has a nucleic acid sequence having at least 97% identity to that of SEQ ID NO: 22. In some embodiments, the miRNA-3G has a nucleic acid sequence having at least 98% identity to that of SEQ ID NO: 22. In some embodiments, the miRNA-3G has a nucleic acid sequence having at least 99% identity to that of SEQ ID NO: 22. In other embodiments, the miRNA-3G has the nucleic acid sequence of SEQ ID NO: 22 (“miRNA734-3G”) (see also FIG. 8).

In some embodiments, the sh734 shRNA is adapted to mimic a miRNA-451 (see SEQ ID NO: 36) structure with a 17 nucleotide base pair stem and a 4-nucleotide loop (miR-451 regulates the drug-transporter protein P-glycoprotein). Notably, this structure does not require processing by DICER. It is believed that the pre-451 mRNA structure is cleaved by Ago2 and subsequently by poly(A)-specific ribonuclease (PARN) to generate the mature miRNA-451 structural mimic (see SEQ ID NO: 37). It is believed that Ago-shRNA mimics of the structure of the endogenous miR-451 and may have the advantage of being DICER independent. This is believed to restrict off target effects of passenger loading, with variable 3′-5′ exonucleolytic activity (23-26 nt mature) (see Herrera-Carrillo, E., and B. Berkhout. 2017. DICER-independent processing of small RNA duplexes: mechanistic insights and applications. Nucleic Acids Res. 45:10369-10379). It is also believed that there exist advantages of utilizing alternate dicer independent processing of shRNAs, including efficient reduced off-target effects of single RNAi-active guide, no saturation of cellular RNAi DICER machinery, and shorter RNA duplexes are less likely to trigger innate RIG-I response.

Alternatives to RNAi

As an alternative to the incorporation of a RNAi, in some embodiments, the expression vectors may include a nucleic acid sequence which encodes antisense oligonucleotides that bind sites in messenger RNA (mRNA). Antisense oligonucleotides of the present disclosure specifically hybridize with a nucleic acid encoding a protein and interfere with transcription or translation of the protein. In some embodiments, an antisense oligonucleotide targets DNA and interferes with its replication and/or transcription. In other embodiments, an antisense oligonucleotide specifically hybridizes with RNA, including pre-mRNA (i.e. precursor mRNA which is an immature single strand of mRNA), and mRNA. Such antisense oligonucleotides may affect, for example, translocation of the RNA to the site of protein translation, translation of protein from the RNA, splicing of the RNA to yield one or more mRNA species, and catalytic activity that may be engaged in or facilitated by the RNA. The overall effect of such interference is to modulate, decrease, or inhibit target protein expression.

In some embodiments, the expression vectors incorporate a nucleic acid sequence encoding for an exon skipping agent or exon skipping transgene. As used herein, the phrase “exon skipping transgene” or “exon skipping agent” refers to any nucleic acid that encodes an antisense oligonucleotide that can generate exon skipping. “Exon skipping” refers to an exon that is skipped and removed at the pre-mRNA level during protein production. It is believed that antisense oligonucleotides may interfere with splice sites or regulatory elements within an exon. This can lead to truncated, partially functional, protein despite the presence of a genetic mutation. Generally, the antisense oligonucleotides may be mutation-specific and bind to a mutation site in the pre-messenger RNA to induce exon skipping.

Exon skipping transgenes encode agents that can result in exon skipping, and such agents are antisense oligonucleotides. The antisense oligonucleotides may interfere with splice sites or regulatory elements within an exon to lead to truncated, partially functional, protein despite the presence of a genetic mutation. Additionally, the antisense oligonucleotides may be mutation-specific and bind to a mutation site in the pre-messenger RNA to induce exon skipping. Antisense oligonucleotides for exon skipping are known in the art and are generally referred to as AONs. Such AONs include small nuclear RNAs (“snRNAs”), which are a class of small RNA molecules that are confined to the nucleus and which are involved in splicing or other RNA processing reactions. Examples of antisense oligonucleotides, methods of designing them, and related production methods are disclosed, for example, in U.S. Publication Nos. 20150225718, 20150152415, 20150140639, 20150057330, 20150045415, 20140350076, 20140350067, and 20140329762, the disclosures of which are each hereby incorporated by reference herein in their entireties.

In some embodiments, the expression vectors of the present disclosure include a nucleic acid which encodes an exon skipping agent which results in exon skipping during the expression of HPRT or which causes an HPRT duplication mutation (e.g. a duplication mutation in Exon 4) (see Baba S, et al. Novel mutation in HPRT1 causing a splicing error with multiple variations. Nucleosides Nucleotides Nucleic Acids. 2017 Jan. 2; 36(1):1-6, the disclosure of which is hereby incorporated by reference herein in its entirety).

In some embodiments, HPRT may be replaced with a modified mutated sequence by spliceosome trans-splicing, thus facilitating knockdown of HPRT. In some embodiments, this (1) requires a mutated coding region to replace the coding sequence in a target RNA, (2) a 5′ or 3′ splice site, and/or (3) a binding domain, i.e., antisense oligonucleotide sequence, which is complementary to the target HPRT RNA. In some embodiments, all three components are required.

WASP Therapeutic Gene

As noted herein, the expression vectors (e.g. the lentiviral expression vectors or AAV vectors) of the present disclosure may also include a second nucleic acid sequence encoding a therapeutic gene (e.g. WASP), whereby the therapeutic gene may correct a defect in a target cell (e.g. HSCs). As will be understood by those in the art, the term “therapeutic gene” includes genomic sequences, cDNA sequences, and smaller engineered gene segments that express, or may be adapted to express, proteins, polypeptides, domains, fusion proteins, and mutants that maintain some or all of the therapeutic function of the full-length polypeptide encoded by the therapeutic gene. Encompassed within the definition of “therapeutic gene” is a “biologically functional equivalent” therapeutic gene. Accordingly, sequences that have about 70% sequence homology to about 99% sequence homology and any range or amount of sequence homology derivable therein, such as, for example, about 70% to about 80%, or about 85% and about 90%; or between about 95% and about 99%; of amino acids that are identical or functionally equivalent to the amino acids of the therapeutic gene will be sequences that are biologically functional equivalents provided the biological activity of the polypeptide is maintained.

In some embodiments, the expression vectors of the present disclosure include a nucleic acid sequence encoding wild-type WASP. By “nucleic acid sequence encoding wild-type WASP,” it is meant that the nucleotide sequence of WASP may (i) be in its natural, non-mutated form (see, e.g., SEQ ID NO: 67); or (ii) differ from its natural, non-mutated form by including one, two, three, or four silent mutations (see, e.g., SEQ ID NO: 68). In some embodiments, the nucleic acid sequence encoding wild-type WASP may include one silent mutation. In other embodiments, the nucleic acid sequence encoding wild-type WASP may include two silent mutations. In yet other embodiments, the nucleic acid sequence encoding wild-type WASP may include three silent mutations. In further embodiments, the nucleic acid sequence encoding wild-type WASP may include four silent mutations. In other embodiments, the expression vectors of the present disclosure include a nucleic acid sequence encoding a codon-optimized WASP. In some embodiments, while the nucleic acid sequences encoding wild-type WASP and codon-optimized WASP are different, they both encode the same protein sequence. In some embodiments, itis believed that the nucleic acid encoding the codon-optimized WASP, in comparison to the nucleic acid encoding wild-type WASP, may offer (i) higher expression, (ii) may lead to comparatively higher transcription levels and therefore comparatively higher Wiskott-Aldrich Syndrome protein levels; and/or iii) other advantages. In some embodiments, the use of a codon-optimized WASP enables the detection of expression of the resulting protein over endogenous WAS.

In some embodiments, the expression vector comprises a nucleic acid sequence encoding a Wiskott-Aldrich syndrome protein, i.e. one that encodes WASP. In some embodiments, the nucleic acid sequence encoding the Wiskott-Aldrich Syndrome protein has a sequence having at least 80% identity to any one of SEQ ID NOS: 1, 2, 3, or 4. In other embodiments, the nucleic acid sequence encoding the Wiskott-Aldrich Syndrome protein has a sequence having at least 85% identity to any one of SEQ ID NOS: 1, 2, 3, or 4. In yet other embodiments, the nucleic acid sequence encoding the Wiskott-Aldrich Syndrome protein has a sequence having at least 90% identity to any one of SEQ ID NOS: 1, 2, 3, or 4. In further embodiments, the nucleic acid sequence encoding the Wiskott-Aldrich Syndrome protein has a sequence having at least 95% identity to any one of SEQ ID NOS: 1, 2, 3, or 4. In further embodiments, the nucleic acid sequence encoding the Wiskott-Aldrich Syndrome protein has a sequence having at least 96% identity to any one of SEQ ID NOS: 1, 2, 3, or 4. In yet further embodiments, the nucleic acid sequence encoding the Wiskott-Aldrich Syndrome protein has a sequence having at least 97% identity to any one of SEQ ID NOS: 1, 2, 3, or 4. In even further embodiments, the nucleic acid sequence encoding the Wiskott-Aldrich Syndrome protein has a sequence having at least 98% identity to any one of SEQ ID NOS: 1, 2, 3, or 4. In even further embodiments, the nucleic acid sequence encoding the Wiskott-Aldrich Syndrome protein has a sequence having at least 99% identity to any one of SEQ ID NOS: 1, 2, 3, or 4. In yet further embodiments, the nucleic acid sequence encoding the Wiskott-Aldrich Syndrome protein has a sequence comprising any one of SEQ ID NOS: 1, 2, 3, or 4.

In some embodiments, the nucleic acid sequence encoding the Wiskott-Aldrich Syndrome protein has a sequence having at least 80% identity to any one of SEQ ID NOS: 67, 68, and 69. In other embodiments, the nucleic acid sequence encoding the Wiskott-Aldrich Syndrome protein has a sequence having at least 85% identity to any one of SEQ ID NOS: 67, 68, and 69. In yet other embodiments, the nucleic acid sequence encoding the Wiskott-Aldrich Syndrome protein has a sequence having at least 90% identity to any one of SEQ ID NOS: 67, 68, and 69. In further embodiments, the nucleic acid sequence encoding the Wiskott-Aldrich Syndrome protein has a sequence having at least 95% identity to any one of SEQ ID NOS: 67, 68, and 69. In further embodiments, the nucleic acid sequence encoding the Wiskott-Aldrich Syndrome protein has a sequence having at least 96% identity to any one of SEQ ID NOS: 67, 68, and 69. In yet further embodiments, the nucleic acid sequence encoding the Wiskott-Aldrich Syndrome protein has a sequence having at least 97% identity to any one of SEQ ID NOS: 67, 68, and 69. In even further embodiments, the nucleic acid sequence encoding the Wiskott-Aldrich Syndrome protein has a sequence having at least 98% identity to any one of SEQ ID NOS: 67, 68, and 69. In even further embodiments, the nucleic acid sequence encoding the Wiskott-Aldrich Syndrome protein has a sequence having at least 99% identity to any one of SEQ ID NOS: 67, 68, and 69. In yet further embodiments, the nucleic acid sequence encoding the Wiskott-Aldrich Syndrome protein has a sequence comprising any one of SEQ ID NOS: 67, 68, and 69.

In some embodiments, the expression vector comprises a nucleic acid which encodes for an amino acid sequence having an identity of at least about 80% to any one of SEQ ID NOS: 5 or 6. In other embodiments, the nucleic acid sequence encodes an amino acid having an identity of at least about 85% to any one of SEQ ID NOS: 5 or 6. In yet other embodiments, the nucleic acid sequence encodes an amino acid having an identity of at least about 90% to any one of SEQ ID NOS: 5 or 6. In further embodiments, the nucleic acid sequence encodes an amino acid having an identity of at least about 95% to any one of SEQ ID NOS: 5 or 6. In further embodiments, the nucleic acid sequence encodes an amino acid having an identity of at least about 96% to any one of SEQ ID NOS: 5 or 6. In yet further embodiments, the nucleic acid sequence encodes an amino acid having an identity of at least about 97% to any one of SEQ ID NOS: 5 or 6. In even further embodiments, the nucleic acid sequence encodes an amino acid having an identity of at least about 98% to any one of SEQ ID NOS: 5 or 6. In even further embodiments, the nucleic acid sequence encodes an amino acid having an identity of at least about 99% to any one of SEQ ID NOS: 5 or 6. In yet further embodiments, the nucleic acid sequence encodes an amino acid comprising any one of SEQ ID NOS: 5 or 6.

Promoters

In some embodiments, different promoters are used to drive expression of each of the nucleic acid sequences incorporated within the disclosed expression vectors. For example, a first nucleic acid sequence encoding an RNAi (e.g. an anti-HPRT shRNA) may be expressed from a first promoter, and a second nucleic acid sequence encoding a therapeutic gene (e.g. a WAS gene) may be expressed from a second promoter, wherein the first and second promoters are different. Likewise, and by way of another example, a first nucleic acid sequence encoding a micro-RNA based shRNA to downregulate HPRT may be expressed from a first promoter and a second nucleic acid sequence encoding a therapeutic gene (e.g. the WAS gene) may be expressed from a second promoter, wherein the first and second promoters are different.

In some embodiments, the promoters may be constitutive promoters or inducible promoters as known to those of ordinary skill in the art. In some embodiments, the promoter includes at least a portion of an HIV LTR (e.g. TAR).

Examples of suitable promoters include, but are not limited to, RNA polymerase I (pol I), polymerase II (pol II), or polymerase III (pol III) promoters. By “RNA polymerase III promoter” or “RNA pol III promoter” or “polymerase III promoter” or “pol III promoter” it is meant any invertebrate, vertebrate, or mammalian promoter, e.g., human, murine, porcine, bovine, primate, simian, etc. that, in its native context in a cell, associates or interacts with RNA polymerase III to transcribe its operably linked gene, or any variant thereof, natural or engineered, that will interact in a selected host cell with an RNA polymerase III to transcribe an operably linked nucleic acid sequence. RNA pol III promoters suitable for use in the expression vectors of the disclosure include, but are not limited, to human U6, mouse U6, and human H1 others.

Examples of pol II promoters include, but are not limited to, Ef1 alpha, CMV, and ubiquitin. Other specific pol II promoters include, but are not limited to, ankyrin promoter (Sabatino D E, et al., Proc Natl Acad Sd USA. (24):13294-9 (2000)), spectrin promoter (Gallagher P G, et al., J Biol Chem. 274(10):6062-73, (2000)), transferrin receptor promoter (Marziali G, et al., Oncogene. 21(52):7933-44, (2002)), band 3/anion transporter promoter (Frazar T F, et al., MoI Cell Biol (14):4753-63, (2003)), band 4.1 promoter (Harrison P R, et al., Exp Cell Res. 155(2):321-44, (1984)), BcI-X1 promoter (Tian C, et al., Blood 15; 101(6):2235-42 (2003)), EKLF promoter (Xue L, et al., Blood. 103(11):4078-83 (2004)). Epub 2004 Feb. 5), ADD2 promoter (Yenerel M N, et al., Exp Hematol. 33(7):758-66 (2005)), DYRK3 promoter (Zhang D, et al., Genomics 85(1): 117-30 (2005)), SOCS promoter (Sarna M K, et al., Oncogene 22(21):3221-30 (2003)), LAF promoter (To M D, et al., bit J Cancer 1; 115(4):568-74, (2005)), PSMA promoter (Zeng H, et al., J Androl (2):215-21, (2005)), PSA promoter (Li H W, et al., Biochem Biophys Res Commun 334(4): 1287-91, (2005)), Probasin promoter (Zhang J, et al., 145(1):134-48, (2004)). Epub 2003 Sep. 18), ELAM-I promoter/E-Selectin (Walton T, et al., Anticancer Res. 18(3A):1357-60, (1998)), Synapsin promoter (Thiel G, et al., Proc Natl Acad Sd USA., 88(8):3431-5(1988)), Willebrand factor promoter (Jahroudi N, Lynch D C. MoI Cell-5zo/.14(2):999-1008, (1994)), FLT1 (Nicklin S A, et al., Hypertension 38(1):65-70, (2001)), Tau promoter (Sadot E, et al., J MoI Biol. 256(5):805-12, (1996)), Tyrosinase promoter (Lillehammer T, et al., Cancer Gene Ther. (2005)), pander promoter (Burkhardt B R, et al., Biochim Biophys Acta. (2005)), neuron-specific enolase promoter (Levy Y S, et al., J Mol Neurosci.21(2):121-32, (2003)), hTERT promoter (Ito H, et al., Hum Gene Ther 16(6):685-98, (2005)), HRE responsive element (Chadderton N, et al., Int J Radiat Oncol Biol Phys.62(1):2U-22, (2005)), lck promoter (Zhang D J, et al., J Immunol. 174(11):6725-31, (2005)), MHCII promoter (De Geest B R, et al., Blood. 101(7):2551-6, (2003), Epub 2002 Nov. 21), and CD1 Ic promoter (Lopez-Rodriguez C, et al., J Biol Chem. 272(46):29120-6 (1997)), the disclosures of which are hereby incorporated by reference herein in their entireties.

In some embodiments, the promoter driving expression of the agent designed to knockdown HPRT is an RNA pol III promoter. In some embodiments, the promoter driving expression of the agent designed to knockdown HPRT is a 7sk promoter (e.g. a 7SK human 7S K RNA promoter). In some embodiments, the 7sk promoter has the nucleic acid sequence provided by ACCESSION AY578685 (Homo sapiens cell-line HEK-293 7SK RNA promoter region, complete sequence, ACCESSION AY578685).

In some embodiments, the 7sk promoter has a nucleic acid sequence having at least 90% identity to that of SEQ ID NO: 28. In some embodiments, the 7sk promoter has a nucleic acid sequence having at least 95% identity to that of SEQ ID NOS: 28. In some embodiments, the 7sk promoter has a nucleic acid sequence having at least 96% identity to that of SEQ ID NOS: 28. In some embodiments, the 7sk promoter has a nucleic acid sequence having at least 97% identity to that of SEQ ID NOS: 28. In some embodiments, the 7sk promoter has a nucleic acid sequence having at least 98% identity to that of SEQ ID NOS: 28. In some embodiments, the 7sk promoter has a nucleic acid sequence having at least 99% identity to that of SEQ ID NOS: 28. In some embodiments, the 7sk promoter has the nucleic acid sequence set forth in SEQ ID NOS: 28.

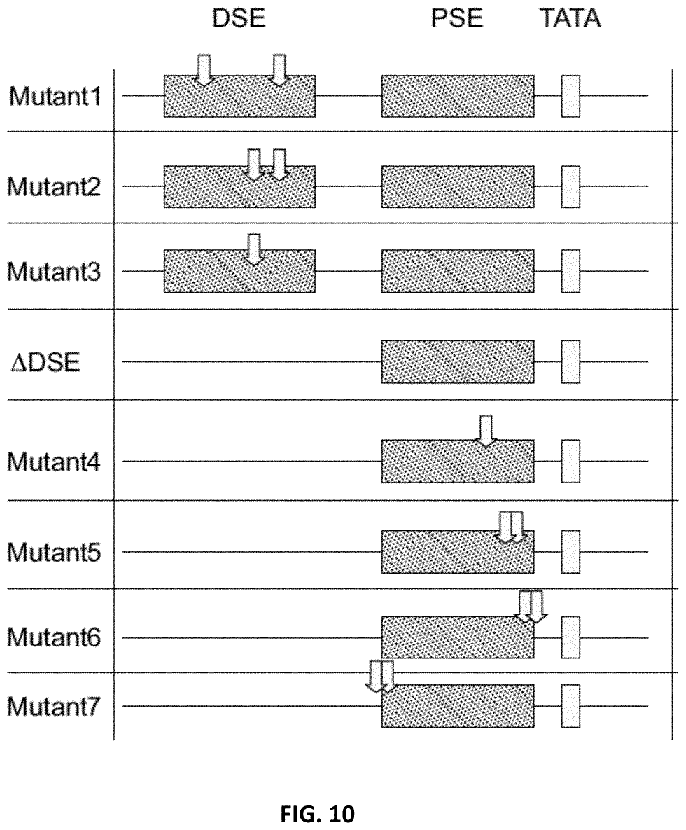

In some embodiments, the 7sk promoter utilized comprises at least one mutation and/or deletion in its nucleic acid sequence in comparison to the 7sk promoter. Suitable 7sk promoter mutations are described in Boyd, D. C., Turner, P. C., Watkins, N.J., Gerster, T. & Murphy, S. Functional Redundancy of Promoter Elements Ensures Efficient Transcription of the Human 7SK Gene in vivo. Journal of Molecular Biology 253, 677-690 (1995), the disclosure of which is hereby incorporated by reference herein in its entirety (see also FIG. 10). In some embodiments, functional mutations or deletions in the 7sk promoter are made in cis-regulatory elements to regulate expression levels of the promoter-driven transgene, including sh734 (see SEQ ID NO: 29). The mutations described are used to establish the correlation between sh734 expression levels driven by the Pol III promoter and to introduce functionality to undergo stable selection in the presence of 6TG therapy and long-term stability and safety. The location of 7sk promoter mutations are depicted in FIG. 10.

In some embodiments, the 7sk promoter has a sequence having at least 95% identity to that of SEQ ID NOS: 29. In some embodiments, the 7sk promoter has a sequence having at least 96% identity to that of SEQ ID NO: 29. In some embodiments, the 7sk promoter has a sequence having at least 97% identity to that of SEQ ID NO: 29. In some embodiments, the 7sk promoter has a sequence having at least 98% identity to that of SEQ ID NO: 29. In some embodiments, the 7sk promoter has a sequence having at least 99% identity to that of SEQ ID NO: 29. In some embodiments, the 7sk promoter has the sequence set forth in SEQ ID NO: 29.

In some embodiments, the promoter that drives expression of a nucleic acid sequence encoding WASP is an MND promoter. Examples of expression cassettes including an MND promoter are illustrated in FIGS. 1A-1E. It is believed that the MND promoter provides a better and more consistent expression in myeloid and lymphoid lineages, especially for WAS.

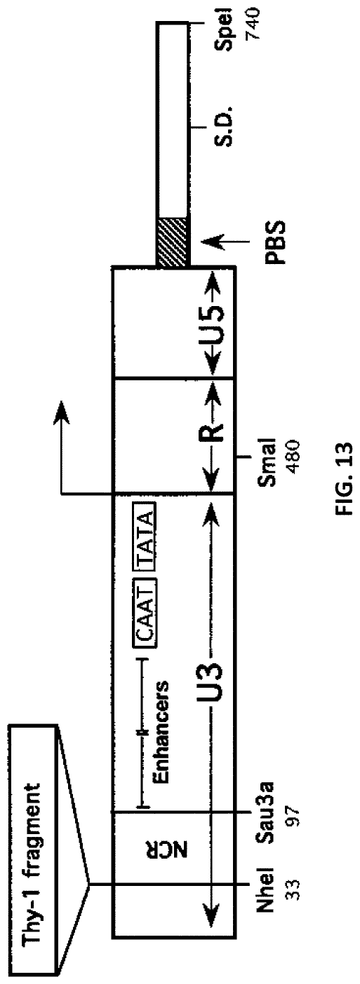

The MND promoter is a synthetic viral promoter. The MND promoter is defined as a “myeloproliferative sarcoma virus enhancer, negative control region deleted, d1587rev primer-binding site substituted.” See Halene, et. al., “Improved Expression in Hematopoietic and Lymphoid Cells in Mice After Transplantation of Bone Marrow Transduced with a Modified Retroviral Vector,” Blood 1999 94:3349-3357 (the disclosure of which is hereby incorporated by reference herein in its entirety). Challita et. al., “Multiple Modifications in cis Elements of the Long Terminal Repeat of Retroviral Vectors Lead to Increased Expression and Decreased DNA Methylation in Embryonic Carcinoma Cells,” Journal of Virology, February 1995, p. 748-755, which is incorporated by reference herein in its entirety, describes the construction of a series of retroviral vectors, containing modifications of the MoMuLV (Moloney murine leukemia virus) transcriptional unit. Such modifications may include (i) substitution of the MoMuLV enhancer to the one from MPSV, (ii) deletion of the NCR, (iii) substitution of the MoMuLV PBS with the PBS of the d1587rev strain, and (iv) insertion of a demethylating fragment cloned from the 59 upstream region of the murine Thy-1 gene. A schematic diagram illustrating the locations of the aforementioned modifications to the Moloney murine leukemia virus transcriptional unit is illustrated in FIG. 13. Other modifications of the MoMuLV (Moloney murine leukemia virus) transcriptional unit have been described. A skilled artisan would understand the “MND promoter” in the context of the present disclosure, encompasses other described modifications or variants of the MoMuLV (Moloney murine leukemia virus) transcriptional unit suitable for driving expression of a nucleic acid sequence encoding WASP. For example, US2017/0051308, which is incorporated by reference herein in its entirety, describes other modifications of the MoMuLV (Moloney murine leukemia virus) transcriptional unit which may be applied to the present disclosure to drive expression of any nucleic acid sequence encoding WASP.

As noted in Challita et. al., “Multiple Modifications in cis Elements of the Long Terminal Repeat of Retroviral Vectors Lead to Increased Expression and Decreased DNA Methylation in Embryonic Carcinoma Cells,” Journal of Virology, February 1995, p. 748-755, an MND promoter may be derived from the modified MoMuLV long terminal repeat. For example, and in some embodiments, an MND promoter used to drive expression of the WASP gene comprises a truncated MND promoter that is truncated in comparison to the MoMuLV long terminal repeat shown in FIG. 13. In some embodiments, the truncation comprises removal of one or more of the regions identified in FIG. 13. In other embodiments, the truncation comprises removal of a portion of one or more of the regions identified in FIG. 13. In some embodiments, the MND promoter used to drive expression of the WASP gene comprises the U3 section of the MoMuLV long terminal repeat. In other embodiments, the MND promoter used to drive expression of the WASP gene comprises a modified U3 region, where the U3 region lacks the negative control region (NCR). In yet other embodiments, the MND promoter used to drive expression of the WASP gene does not include the PBS region, the R region, or the U5 region of the MoMuLV long terminal repeat but includes at least a portion of the U3 region. In further embodiments, the MND promoter used to drive expression of the WASP gene comprises a modified U3 region, where the U3 region lacks the negative control region (NCR); and where the MND promoter does not include the PBS region, the R region, or the U5 region of the MoMuLV long terminal repeat. In even further embodiments, the MND promoter used to drive expression of the WASP gene does not include the PBS region of the MoMuLV long terminal repeat.

In some embodiments, the MND promoter has a nucleic acid sequence having at least 90% identity to any one of SEQ ID NOS: 7, 8, 9, 10, 11, or 12. In other embodiments, the MND promoter has a nucleic acid sequence having at least 95% identity to any one of SEQ ID NOS: 7, 8, 9, 10, 11, or 12. In yet other embodiments, the MND promoter has a nucleic acid sequence having at least 97% identity to any one of SEQ ID NOS: 7, 8, 9, 10, 11, or 12. In further embodiments, the MND promoter has a nucleic acid sequence having at least 98% identity to any one of SEQ ID NOS: 7, 8, 9, 10, 11, or 12. In even further embodiments, the MND promoter has a nucleic acid sequence having at least 99% identity to any one of SEQ ID NOS: 7, 8, 9, 10, 11, or 12. In yet further embodiments, the MND promoter comprises the nucleic acid sequence of any one of SEQ ID NOS: 6, 7, 8, 9, 10, 11, or 12.

Without wishing to be bound by any particular theory, it is believed that an MND promoter may drive more efficient expression of WASP as compared with other promoters, such as a WS1.6 promoter. It is believed that the WS1.6 promoter (a section of an endogenous WAS promoter) is less active in primary human cells compared with MND. It is also believed that WS1.6-huWASp LV mediates limited WASP expression in vivo. It is also believed that transplantation of stem cells transduced with MND-huWASp LV may result in sustained, endogenous levels of WASP in all hematopoietic lineages, progressive selection for WASP+T, natural killer T and B cells, rescue of T-cell proliferation and cytokine production, and/or substantial restoration of marginal zone (MZ) B cells. In view of the foregoing, it is believed that the MND may provide the most efficacious results.

Insulators

In some embodiments, the expression vectors of the present disclosure include an insulator, e.g. a chromatin insulator. Without wishing to be bound by any particular theory, it is believed that chromatin insulator elements prevent the spread of heterochromatin and silencing of genes, reduces chromatin position effects, and has enhancer blocking activity. It is believed that these properties are desirable for consistent predictable expression and safe transgene delivery with randomly integrating vectors. It is also believed that overcoming chromatin position effects can reduce the number of copies required for a therapeutic effect and reduce the risk of genotoxicity of vectors. Studies have shown that insulated vectors showed consistent, predictable expression, regardless of integration site in the differentiated progeny of hematopoietic stem cells, resulting in about 2 to about 4-fold higher overall expression. Recent evidence also suggests that cHS4 insulated lentivirus vectors may reduce the risk of insertional activation of cellular oncogenes. Despite the beneficial effects of insulated vectors, they also lead to a significant reduction in titers with insertion of the full-length 1.2 Kb cHS4 insulator element in the 3′LTR of lentivirus vectors. There are similar reports of lowering of viral titers or unstable transmission with gamma-retrovirus vectors containing insertions in the 3′ LTR. This reduction in titer is believed to become practically limiting for scale-up of vector production for clinical trials, especially with vectors carrying relatively large expression cassettes. It is believed that these results have important implications for vector design for clinical gene therapy. Studies on the chicken hypersensitive site-4 (cHS4) element, a prototypic insulator, have identified CTCF and USF-1/2 motifs in the proximal 250 bp of cHS4, termed the “core”, which provide enhancer blocking activity and reduce position effects. However, the core alone does not insulate viral vectors effectively. While the full-length cHS4 is believed to have excellent insulating properties, its large size severely compromises vector titers.

In some embodiments, the titer of lentiviral vectors is increased by incorporating one or more reduced-length chromatin insulators containing functional portions of a full-length chromatin insulator. In some embodiments, the functional reduced-length chromatin insulator is derived from a chicken hypersensitive site-4 (cHS4) element. In some embodiments, the functional reduced-length insulator is a cHS4-derived insulator of 650 base pairs or less. In some embodiments, the insulator is a 650cHS4, a 400cHS4, or a foamy virus insulator. In some embodiments, the functional portions are derived from one type of full-length chromatin insulator. In some embodiments, the reduced-length functional insulator comprises functional portions of two or more separate variants of chromatin insulators. In some embodiments, the insulator has a nucleic acid sequence having at least 90% identity to any one of SEQ ID NOS: 38, 39, or 40. In other embodiments, the insulator has a nucleic acid sequence having at least 95% identity to any one of SEQ ID NOS; 38, 39, or 40. In yet other embodiments, the insulator has a nucleic acid sequence having at least 97% identity to any one of SEQ ID NOS: 38, 39, or 40. In further embodiments, the insulator has the nucleic acid sequence of any one of SEQ ID NOS: 38, 39, or 40. Other insulators are described in U.S. Patent Publication Nos. 2018/0142255 and 2007/0154456, the disclosures of which are hereby incorporated by reference herein in their entireties.

It is believed that chromatin insulators offer two levels of activity. It is believed that they prevent epigenetic silencing of the integrated sequences. It is believed that epigenetic silencing of parts of the genome is a process which occurs during differentiation of HSCs into cell lineages. As an example, WASP could be well expressed in transduced HSCs. It is believed, however, that once HSCs give rise to differentiated cells, a lower WASP expression may be achieved due to these silencing events. It is also believed that insulators prevent promoter activity outside of the integration site (enhancer blocking activity). For example, if a lentivirus integrates “in” or “near” a particular gene (“gene x”), it prevents a promoter, such as an MND, to function on WASP and on the “gene x” located around the integration site.

Production of Vectors

In some embodiments, an expression cassette, such as one having a particular transgene for expression (e.g. SEQ ID NO: 15), is inserted into expression vector, such as a lentiviral expression vector, to provide for a vector having at least one transgene for expression. In some embodiments, the lentiviral expression vector may be selected from the group consisting of pTL20c, pTL20d, FG, pRRL, pCL20, pLKO.1 puro, pLKO.1, pLKO.3G, Tet-pLKO-puro, pSico, pLJM1-EGFP, FUGW, pLVTHM, pLVUT-tTR-KRAB, pLL3.7, pLB, pWPXL, pWPI, EF.CMV.RFP, pLenti CMV Puro DEST, pLenti-puro, pLOVE, pULTRA, pLJM1-EGFP, pLX301, pInducer20, pHIV-EGFP, Tet-pLKO-neo, pLV-mCherry, pCW57.1, pLionII, pSLIK-Hygro, and pInducer10-mir-RUP-PheS. In other embodiments, the lentiviral expression vector may be selected from AnkT9W vector, a T9Ank2W vector, a TNS9 vector, a lentiglobin HPV569 vector, a lentiglobin BB305 vector, a BG-1 vector, a BGM-1 vector, a d432βAγ vector, a mLAβΔγV5 vector, a GLOBE vector, a G-GLOBE vector, a βAS3-FB vector, a V5 vector, a V5m3 vector, a V5m3-400 vector, a G9 vector, and a BCL11A shmir vector. In some embodiments, the lentiviral expression vector may be selected from the group consisting pTL20c, pTL20d, FG, pRRL and pCL20. In still other embodiments, the lentiviral expression vector is pTL20c.

For example, an expression cassette having a transgene for expression (e.g. a WAS transgene) may be inserted into a pTL20c vector (SEQ ID NO: 18) according to the methods described in in United States Patent Publication No. 2018/0112233, the disclosure of which is hereby incorporated by reference herein in its entirety. This “intermediate” is illustrated in FIG. 1A. In some embodiments, the pTL20c vector includes a vector backbone having a nucleic acid sequence having at least 95% identity to that of SEQ ID NO: 30. In some embodiments, the pTL20c vector includes a vector backbone having a nucleic acid sequence having at least 90% identity to that of SEQ ID NO: 30.

In some embodiments, following insertion of the expression cassette into the expression vector, a second expression cassette is inserted into the vector having a second transgene for expression. For example, an expression cassette including a nucleic acid sequence to knockdown HPRT (e.g. SEQ ID NO: 14) may be inserted into the vector having the at least one transgene for expression.

In other embodiments, the expression cassette may comprise a first transgene for expression operably linked to a promoter, and at least one second transgene for expression operably linked to a second promoter. For example, the expression cassette for insertion into the expression vector may include a first transgene for expression, and a second nucleic acid sequence to knockdown HPRT (e.g. SEQ ID NO: 14), for example the 3.2 kb hWASWT cassette (SEQ ID NO: 58) or 3.2 kb hWAS^{CO} cassette (SEQ ID NO: 59). In some embodiments, the expression cassette comprises a first transgene for expression and a second nucleic acid sequence 7sk/sh734 (e.g. SEQ ID NO: 14). In some embodiments, the expression cassette comprises a WAS transgene for expression and a second nucleic acid sequence 7sk/sh734 (e.g. SEQ ID NO: 14), for example the 3.2 kb hWASWT cassette (SEQ ID NO: 58) or 3.2 kb hWAS^{CO} cassette (SEQ ID NO: 59). In some embodiments, the expression cassette comprises a WAS transgene operably linked to a promoter and a second nucleic acid sequence to knockdown HPRT operably linked to a promoter, for example the 3.2 kb hWASWT cassette (SEQ ID NO: 58) or 3.2 kb hWAS^{CO} cassette (SEQ ID NO: 59). In some embodiments, the expression cassette comprises a WAS transgene operably linked to an MND promoter and 7sk/sh734 (e.g. SEQ ID NO: 14), for example the 3.2 kb hWASWT cassette (SEQ ID NO: 58) or 3.2 kb hWAS^{CO} cassette (SEQ ID NO: 59). In some embodiments, the expression cassette comprising a first and second transgene may be inserted into an expression vector. For example, in some embodiments, the expression cassette comprising a first and second transgene may be inserted into an expression vector in a single step.

Non-limiting examples of resulting vectors are depicted in FIGS. 1B through 1E. As illustrated in FIGS. 1B-1E, the 7sk expression cassette (e.g. an expression cassette having SEQ ID NO: 14) may be inserted into an expression vector in different locations (e.g. different locations relative to a WASP expression cassette). In addition, the 7sk expression cassette may be inserted into an expression vector in different orientations (compare, for example, the orientations of the 7sk promoter between FIG. 1B vs. 1C, and between FIGS. 1D and 1E). It is believed that the different locations and/or orientations of the 7sk expression cassette relative to the WASP expression cassette may enhance expression of sh734. Additional examples of different configurations are set forth below:

TABLE 1
Various configurations of different vectors.

| | | | shRNA and Location/Orientation to WAS transgene | | | |
|---|---|---|---|---|---|---|
| SEQ ID NO: | Promoter | cDNA | Type | Location | Orientation | Insulator |
| 42 | MND | Wild-type | 7SK/sh734 | Upstream | Forward | 400ins |
| 43 | MND | Wild-type | 7SK/sh734 | Upstream | Reverse | 400ins |
| 44 | MND | Wild-type | 7SK/sh734 | Downstream | Forward | 400ins |
| 45 | MND | Wild-type | 7SK/sh734 | Downstream | Reverse | 400ins |
| 46 | MND | Codon-optimized | 7SK/sh734 | Upstream | Forward | 400ins |
| 47 | MND | Codon-optimized | 7SK/sh734 | Upstream | Reverse | 400ins |
| 48 | MND | Codon-optimized | 7SK/sh734 | Downstream | Forward | 400ins |
| 49 | MND | Codon-optimized | 7SK/sh734 | Downstream | Reverse | 400ins |
| 50 | MND | Wild-type | 7SK/sh734 | Upstream | Forward | 650ins |
| 51 | MND | Wild-type | 7SK/sh734 | Downstream | Forward | 650ins |
| 52 | MND | Wild-type | 7SK/sh734 | Upstream | Reverse | 650ins |
| 53 | MND | Wild-type | 7SK/sh734 | Downstream | Reverse | 650ins |
| 54 | MND | Codon-optimized | 7SK/sh734 | Upstream | Forward | 650ins |
| 55 | MND | Codon-optimized | 7SK/sh734 | Downstream | Forward | 650ins |
| 56 | MND | Codon-optimized | 7SK/sh734 | Upstream | Reverse | 650ins |
| 57 | MND | Codon-optimized | 7SK/sh734 | Downstream | Reverse | 650ins |

In some embodiments, the WASP expression cassette is located upstream relative to the 7sk/sh734 expression cassette.

In some embodiments, the WASP expression cassette is located downstream relative to the 7sk/sh734 expression cassette.

In some embodiments, the 7SK/sh734 expression cassette and the WASP expression cassette are oriented in the same direction.

In some embodiments, the 7SK/sh734 expression cassette and the WASP expression cassette are oriented in opposing directions.

In some embodiments, the 7SK/sh734 expression cassette is oriented in a forward direction relative to the WASP cassette.

In some embodiments, the 7SK/sh734 expression cassette is oriented in a reverse direction relative to the WASP expression cassette.

In some embodiments, the 7SK/sh734 expression cassette is located upstream and oriented in a forward direction relative to the WASP cassette.

In some embodiments, the 7SK/sh734 expression cassette is located upstream and oriented in a reverse direction relative to the WASP expression cassette.

In some embodiments, the 7SK/sh734 expression cassette is located downstream and oriented in a forward direction relative to the WASP cassette.

In some embodiments, the 7SK/sh734 expression cassette is located downstream and oriented in a reverse direction relative to the WASP expression cassette.

In some embodiments, the first transgene expression cassette is located upstream relative to the 7sk/sh734 expression cassette.

In some embodiments, the first transgene expression cassette is located downstream relative to the 7sk/sh734 expression cassette.

In some embodiments, the 7SK/sh734 expression cassette and the first transgene expression cassette are oriented in the same direction.

In some embodiments, the 7SK/sh734 expression cassette and the first transgene expression cassette are oriented in opposing directions.

In some embodiments, the 7sk/sh734 expression cassette is oriented in a forward direction relative to the first transgene expression cassette.

In some embodiments, the 7SK/sh734 expression cassette is oriented in a reverse direction relative to the first transgene expression cassette.

In some embodiments, the 7SK/sh734 expression cassette is located upstream and oriented in a forward direction relative to the first transgene expression cassette.

In some embodiments, the 7SK/sh734 expression cassette is located upstream and oriented in a reverse direction relative to the first transgene expression cassette.

In some embodiments, the 7SK/sh734 expression cassette is located downstream and oriented in a forward direction relative to the first transgene expression cassette.

In some embodiments, the 7SK/sh734 expression cassette is located downstream and oriented in a reverse direction relative to the first transgene expression cassette.

In yet other embodiments, the expression cassette may comprise a first transgene for expression operably linked to a promoter, and at least one second nucleic acid sequence 7sk/sh734 (e.g. SEQ ID NO: 14). In some embodiments, the expression cassette may comprise a first transgene for expression operably linked to a promoter, and at least two nucleic acid sequences 7sk/sh734 (e.g. SEQ ID NO: 14). In some embodiments, the expression cassette comprises a WAS transgene for expression and at least two 7SK/sh734 nucleic acid sequences (e.g. SEQ ID NO: 14).

Additional examples of suitable expression cassettes comprising a first transgene for expression operably linked to a promoter, and at least one second transgene for expression operably linked to a second promoter include:

$$7 SK / sh {734}_{-} MND / {hWASWT}_{-} {WPRE}_{-} 7 SK / sh 734 ;$$

$$SK / sh {734}_{-} {MND}_{-} {hWASCO}_{-} {WPRE}_{-} r7 SK / sh 734 ;$$

$$r7 SK / sh {734}_{-} MND / {hWASWT}_{-} {WPRE}_{-} r7 SK / sh 734 ; and$$

$$7 SK / sh {734}_{-} MND / {hWASCO}_{-} {WPRE}_{-} 7 SK / sh 734.$$

In some embodiments, the expression cassette may comprise a first transgene for expression operably linked to a promoter, and at least two nucleic acid sequences to knockdown HPRT (e.g. SEQ ID NO: 14), for example:

7SK/sh734_MND/hWASWT_WPRE_7SK/sh734;

r7SK/sh734_MND_hWASCO_WPRE_r7SK/sh734;

r7SK/sh734_MND/hWASWT_WPRE_r7SK/sh734; or

7SK/sh734_MND/hWASCO_WPRE_7SK/sh734.

A skilled artisan will appreciate that expression cassettes of this type, comprising and at least two nucleic acid sequences to knockdown HPRT may provide further access to a range of constructs with differing location and/or orientation of the 7SK/sh734 expression cassette by selective removal of one of the two nucleic acid sequences to knockdown HPRT.

In some embodiments, the expression cassette may be inserted into an expression vector. For example, the expression cassette comprising a first and second transgene may be inserted into an expression vector in a single step, for example the 3.2 kb hWASWT cassette (SEQ ID NO: 58) or 3.2 kb hWAS^{CO} cassette (SEQ ID NO: 59). Further examples of configurations are set forth in Example 1 and Table 10, herein.

It will be appreciated by a skilled artisan, that an expression cassette of the type described in SEQ ID NO: 58 or SEQ ID NO: 59 comprising a first transgene for expression operably linked to a promoter, and at least one second transgene for expression operably linked to a second promoter, may be further modified or derivatized prior to insertion into an expression vector, after insertion into an expression vector, or combinations thereof. For example, it is envisaged that the expression cassette comprising a first transgene for expression operably linked to a promoter and at least one second transgene for expression operably linked to a second promoter may be further modified or derivatized prior to insertion into an expression vector to i) add one or more additional transgenes or genetic elements, ii) remove one or more transgenes or genetic elements, iii) replace one or more of the transgenes or genetic elements with one or more alternative transgenes or genetic elements, or combinations thereof.

In some embodiments, the 3.2 kb hWASWT cassette (SEQ ID NO: 58) or 3.2 kb hWAS^{CO} cassette (SEQ ID NO: 59) may be modified or derivatized as described above. In some embodiments, the WAS transgene of the 3.2 kb hWASWT cassette or 3.2 kb hWAS^{CO} (SEQ ID NOS: 58 or 59) may be replaced with an alternative transgene for expression, to provide an expression cassette for insertion into the expression vector comprising a transgene for expression other than the WAS transgene, and a second nucleic acid sequence to knockdown HPRT (e.g. SEQ ID NO: 14). In still other embodiments, at least one of the two 7SK/sh734 nucleic acid sequences of the 3.2 kb hWASWT cassette or 3.2 kb hWAS^{CO} (SEQ ID NOS: 58 or 59) may be replaced with an alternative transgene for expression, to provide an expression cassette for insertion into the expression vector comprising an alternative transgene for expression other than 7SK/sh734, and the WAS transgene. In other embodiments, each of the 7SK/sh734 nucleic acid sequences and the WAS transgene of the 3.2 kb hWASWT cassette or 3.2 kb hWAS^{CO} (SEQ ID NOS: 58 or 59) may be replaced with an alternative transgenes for expression, to provide an expression cassette for insertion into the expression vector comprising alternative transgenes for expression, other than 7SK/sh734 and the WAS transgene. Specifically, the first transgene for expression and the second transgene for expression in an expression cassette such as the 3.2 kb hWASWT cassette (SEQ ID NO: 58) or 3.2 kb hWAS^{CO} cassette (SEQ ID NO: 59) may be replaced by alternative transgenes, each of which may be the same or different. An expression cassette derived from the 3.2 kb hWASWT cassette or the 3.2 kb hWAS^{CO} (SEQ ID NOS: 58 or 59) comprising a third transgene for expression operably linked to a third promoter, and at least one fourth transgene for expression operably linked to a fourth promoter is thus provided.

Alternatively, it is envisaged that the expression cassette comprising a first transgene for expression operably linked to a promoter and at least one second transgene for expression operably linked to a second promoter may be further modified or derivatized after insertion into an expression vector to provide i) add one or more additional transgenes or genetic elements, ii) remove one or more transgenes or genetic elements, iii) replace one or more of the transgenes or genetic elements with alternative transgenes or genetic elements, or combinations thereof.

In some embodiments, the 3.2 kb hWASWT cassette or 3.2 kb hWAS^{CO} cassette may be modified or derivatized after insertion into an expression vector, for example, in intermediate vectors of the type described by SEQ ID NOS: 63, 64, 65 or 66, as described above. In some embodiments, the wild-type WAS transgene of the 3.2 kb hWASWT cassette or the codon optimized WAS transgene of the 3.2 kb hWAS^{CO} cassette in any one of intermediate vectors SEQ ID NOS: 63, 64, 65 or 66 may be replaced with an alternative transgene for expression, to provide an expression vector comprising a transgene for expression other than the WAS transgene, and a second nucleic acid sequence to knockdown HPRT (e.g. SEQ ID NO: 14). In still other embodiments, at least one of the two 7SK/sh734 nucleic acid sequences of the 3.2 kb hWASWT cassette or 3.2 kb hWAS^{CO} (SEQ ID NOS: 58 or 59) in intermediate vectors of the type described by SEQ ID NOS: 63, 64, 65 or 66 may be replaced with an alternative transgene for expression, to provide an expression vector comprising an alternative transgene for expression, other than 7SK/sh734. In other embodiments, each of the 7SK/sh734 nucleic acid sequences and the WAS transgene of the 3.2 kb hWASWT or 3.2 kb hWAS^{CO} cassette in intermediate vectors of the type described by SEQ ID NOS: 63, 64, 65 or 66 may be replaced with alternative transgenes for expression, to provide an expression vector comprising alternative transgenes for expression, other than 7SK/sh734 and the WAS transgene. Specifically, the first transgene for expression and the second transgene for expression in an expression cassette, such as the 3.2 kb hWASWT or 3.2 kb hWAS^{CO} cassette, in intermediate vectors of the type described by SEQ ID NOS: 63, 64, 65 or 66 may be replaced with alternative transgenes, each of which may be the same or different. An expression vector, derived from intermediate vectors of the type described by SEQ ID NOS: 63, 64, 65 or 66, comprising a third transgene for expression operably linked to a third promoter, and at least one fourth transgene for expression operably linked to a fourth promoter is thus provided.

Polynucleotides

The present disclosure also provides polynucleotides having at least 90% sequence identity to any one of SEQ ID NOS: 42-57.

In some embodiments, the polynucleotide has 90% identity to that of SEQ ID NO: 42. In some embodiments, the polynucleotide has 95% identity to that of SEQ ID NO: 42. In some embodiments, the polynucleotide has 96% identity to that of SEQ ID NO: 42. In some embodiments, the polynucleotide has 97% identity to that of SEQ ID NO: 42. In some embodiments, the polynucleotide has 98% identity to that of SEQ ID NO: 42. In some embodiments, the polynucleotide has 99% identity to that of SEQ ID NO: 42. In some embodiments, the polynucleotide comprises the nucleic acid sequence of SEQ ID NO: 42.

In some embodiments, the polynucleotide has 90% identity to that of SEQ ID NO: 43. In some embodiments, the polynucleotide has 95% identity to that of SEQ ID NO: 43. In some embodiments, the polynucleotide has 96% identity to that of SEQ ID NO: 43. In some embodiments, the polynucleotide has 97% identity to that of SEQ ID NO: 43. In some embodiments, the polynucleotide has 98% identity to that of SEQ ID NO: 43. In some embodiments, the polynucleotide has 99% identity to that of SEQ ID NO: 43. In some embodiments, of the polynucleotide comprises the nucleic acid sequence of SEQ ID NO: 43.

In some embodiments, the polynucleotide has 90% identity to that of SEQ ID NO: 44. In some embodiments, the polynucleotide has 95% identity to that of SEQ ID NO: 44. In some embodiments, the polynucleotide has 96% identity to that of SEQ ID NO: 44. In some embodiments, the polynucleotide has 97% identity to that of SEQ ID NO: 44. In some embodiments, the polynucleotide has 98% identity to that of SEQ ID NO: 44. In some embodiments, the polynucleotide has 99% identity to that of SEQ ID NO: 44. In some embodiments, the polynucleotide comprises the nucleic acid sequence of SEQ ID NO: 44.

In some embodiments, the polynucleotide has 90% identity to that of SEQ ID NO: 45. In some embodiments, the polynucleotide has 95% identity to that of SEQ ID NO: 45. In some embodiments, the polynucleotide has 96% identity to that of SEQ ID NO: 45. In some embodiments, the polynucleotide has 97% identity to that of SEQ ID NO: 45. In some embodiments, the polynucleotide has 98% identity to that of SEQ ID NO: 45. In some embodiments, the polynucleotide has 99% identity to that of SEQ ID NO: 45. In some embodiments, the polynucleotide comprises the nucleic acid sequence of SEQ ID NO: 45.

In some embodiments, the polynucleotide has 90% identity to that of SEQ ID NO: 46. In some embodiments, the polynucleotide has 95% identity to that of SEQ ID NO: 46. In some embodiments, the polynucleotide has 96% identity to that of SEQ ID NO: 46. In some embodiments, the polynucleotide has 97% identity to that of SEQ ID NO: 46. In some embodiments, the polynucleotide has 98% identity to that of SEQ ID NO: 46. In some embodiments, the polynucleotide has 99% identity to that of SEQ ID NO: 46. In some embodiments, the polynucleotide comprises the nucleic acid sequence of SEQ ID NO: 46.

In some embodiments, the polynucleotide has 90% identity to that of SEQ ID NO: 47. In some embodiments, the polynucleotide has 95% identity to that of SEQ ID NO: 47. In some embodiments, the polynucleotide has 96% identity to that of SEQ ID NO: 47. In some embodiments, the polynucleotide has 97% identity to that of SEQ ID NO: 47. In some embodiments, the polynucleotide has 98% identity to that of SEQ ID NO: 47. In some embodiments, the polynucleotide has 99% identity to that of SEQ ID NO: 47. In some embodiments, the polynucleotide comprises the nucleic acid sequence of SEQ ID NO: 47.

In some embodiments, the polynucleotide has 90% identity to that of SEQ ID NO: 48. In some embodiments, the polynucleotide has 95% identity to that of SEQ ID NO: 48. In some embodiments, the polynucleotide has 96% identity to that of SEQ ID NO: 48. In some embodiments, the polynucleotide has 97% identity to that of SEQ ID NO: 48. In some embodiments, the polynucleotide has 98% identity to that of SEQ ID NO: 48. In some embodiments, the polynucleotide has 99% identity to that of SEQ ID NO: 48. In some embodiments, the polynucleotide comprises the nucleic acid sequence of SEQ ID NO: 48.

In some embodiments, the polynucleotide has 90% identity to that of SEQ ID NO: 49. In some embodiments, the polynucleotide has 95% identity to that of SEQ ID NO: 49. In some embodiments, the polynucleotide has 96% identity to that of SEQ ID NO: 49. In some embodiments, the polynucleotide has 97% identity to that of SEQ ID NO: 49. In some embodiments, the polynucleotide has 98% identity to that of SEQ ID NO: 49. In some embodiments, the polynucleotide has 99% identity to that of SEQ ID NO: 49. In some embodiments, the polynucleotide comprises the nucleic acid sequence of SEQ ID NO: 49.

In some embodiments, the polynucleotide has 90% identity to that of SEQ ID NO: 50. In some embodiments, the polynucleotide has 95% identity to that of SEQ ID NO: 50. In some embodiments, the polynucleotide has 96% identity to that of SEQ ID NO: 50. In some embodiments, the polynucleotide has 97% identity to that of SEQ ID NO: 50. In some embodiments, the polynucleotide has 98% identity to that of SEQ ID NO: 50. In some embodiments, the polynucleotide has 99% identity to that of SEQ ID NO: 50. In some embodiments, the polynucleotide comprises the nucleic acid sequence of SEQ ID NO: 50 (see also FIG. 1F). In some embodiments, the polynucleotide of SEQ ID NO: 50 comprises the components identified in Table 2:

TABLE 2
Elements of the vector
pBRNGTR20_pTL20c_SK734fwd_MND_WAS_650
(“pBRNGTR20”).

| | | Start | End |
|---|---|---|---|
| Element | Orientation | (nt position) | (nt position) |
| 7tetO promoter | forward | 28 | 315 |
| 7SK promoter | forward | 2396 | 2644 |
| shRNA 734 | forward | 2645 | 2691 |
| MND promoter | forward | 2710 | 3056 |
| WAS cDNA (wild-type ORF) | forward | 3098 | 4606 |
| WPRE | forward | 4615 | 5204 |
| rcHS4 Ins-650 insulator | forward | 5340 | 5998 |
| rabbit beta-globin | forward | 6122 | 6570 |
| polyadenylation signal | | | |

In some embodiments, the polynucleotide has 90% identity to that of SEQ ID NO: 51. In some embodiments, the polynucleotide has 95% identity to that of SEQ ID NO: 51. In some embodiments, the polynucleotide has 96% identity to that of SEQ ID NO: 51. In some embodiments, the polynucleotide has 97% identity to that of SEQ ID NO: 51. In some embodiments, the polynucleotide has 98% identity to that of SEQ ID NO: 51. In some embodiments, the polynucleotide has 99% identity to that of SEQ ID NO: 51. In some embodiments, the polynucleotide comprises the nucleic acid sequence of SEQ ID NO: 51 (see also FIG. 1G). In some embodiments, the polynucleotide of SEQ ID NO: 51 comprises the components identified in Table 3:

TABLE 3
Elements of the vector
pBRNGTR21_pTL20c_MND_WAS_SK734fwd_650
(“pBRNGTR21”).

| | | Start | End |
|---|---|---|---|
| Element | Orientation | (nt position) | (nt position) |
| 7tetO promoter | forward | 28 | 315 |
| MND promoter | forward | 2402 | 2748 |
| WAS cDNA (wild-type ORF) | forward | 2790 | 4298 |
| WPRE | forward | 4307 | 4896 |
| 7SK promoter | forward | 4909 | 5157 |
| shRNA 734 | forward | 5158 | 5204 |
| rcHS4 Ins-650 insulator | forward | 5340 | 5998 |
| rabbit beta-globin | forward | 6122 | 6570 |
| polyadenylation signal | | | |

In some embodiments, the polynucleotide has 90% identity to that of SEQ ID NO: 52. In some embodiments, the polynucleotide has 95% identity to that of SEQ ID NO: 52. In some embodiments, the polynucleotide has 96% identity to that of SEQ ID NO: 52. In some embodiments, the polynucleotide has 97% identity to that of SEQ ID NO: 52. In some embodiments, the polynucleotide has 98% identity to that of SEQ ID NO: 52. In some embodiments, the polynucleotide has 99% identity to that of SEQ ID NO: 52. In some embodiments, the polynucleotide comprises the nucleic acid sequence of SEQ ID NO: 52 (see also FIG. 1H). In some embodiments, the polynucleotide of SEQ ID NO: 52 comprises the components identified in Table 4:

TABLE 4
Elements of the vector
pBRNGTR22_pTL20c_SK734rev_MND_WAS_650
(“pBRNGTR22”).

| | | Start | End |
|---|---|---|---|
| Element | Orientation | (nt position) | (nt position) |
| 7tetO promoter | forward | 28 | 315 |
| shRNA 734 | reverse | 2402 | 2448 |
| 7SK promoter | reverse | 2449 | 2697 |
| MND promoter | forward | 2710 | 3056 |
| WAS cDNA (wild-type ORF) | forward | 3098 | 4606 |
| WPRE | forward | 4615 | 5204 |
| rcHS4 Ins-650 insulator | forward | 5340 | 5998 |
| rabbit beta-globin | forward | 6122 | 6570 |
| polyadenylation signal | | | |

In some embodiments, the polynucleotide has 90% identity to that of SEQ ID NO: 53. In some embodiments, the polynucleotide has 95% identity to that of SEQ ID NO: 53. In some embodiments, the polynucleotide has 96% identity to that of SEQ ID NO: 53. In some embodiments, the polynucleotide has 97% identity to that of SEQ ID NO: 53. In some embodiments, the polynucleotide has 98% identity to that of SEQ ID NO: 53. In some embodiments, the polynucleotide has 99% identity to that of SEQ ID NO: 53. In some embodiments, the polynucleotide comprises the nucleic acid sequence of SEQ ID NO: 53 (see also FIG. 1I). In some embodiments, the polynucleotide of SEQ ID NO: 53 comprises the components identified in Table 5:

TABLE 5
Elements of the vector
pBRNGTR23_pTL20c_MND_WAS_SK734rev_650
(“pBRNGTR23”).

| | | Start | End |
|---|---|---|---|
| Element | Orientation | (nt position) | (nt position) |
| 7tetO promoter | forward | 28 | 315 |
| MND promoter | forward | 2402 | 2748 |
| WAS cDNA (wild-type ORF) | forward | 2790 | 4298 |
| WPRE | forward | 4307 | 4896 |
| shRNA 734 | reverse | 4915 | 4961 |
| 7SK promoter | reverse | 4962 | 5210 |
| rcHS4 Ins-650 insulator | forward | 5340 | 5998 |
| rabbit beta-globin | forward | 6122 | 6570 |
| polyadenylation signal | | | |

In some embodiments, the polynucleotide has 90% identity to that of SEQ ID NO: 54. In some embodiments, the polynucleotide has 95% identity to that of SEQ ID NO: 4. In some embodiments, the polynucleotide has 96% identity to that of SEQ ID NO: 54. In some embodiments, of the polynucleotide has 97% identity to that of SEQ ID NO: 54. In some embodiments, the polynucleotide has 98% identity to that of SEQ ID NO: 4. In some embodiments, the polynucleotide has 99% identity to that of SEQ ID NO: 54. In some embodiments, the polynucleotide comprises the nucleic acid sequence of SEQ ID NO: 54 (see also FIG. 1J). In some embodiments, the polynucleotide of SEQ ID NO: 54 comprises the components identified in Table 6:

TABLE 6
Elements of the vector
pTL20c_SK734fwd_MND_coWAS_650
(“pBRNGTR24”).

| | | Start | End |
|---|---|---|---|
| Element | Orientation | (nt position) | (nt position) |
| 7tetO promoter | forward | 28 | 315 |
| 7SK promoter | forward | 2396 | 2644 |
| shRNA 734 | forward | 2645 | 2691 |
| MND promoter | forward | 2710 | 3056 |
| WAS cDNA | forward | 3098 | 4606 |
| (codon optimized ORF) | | | |
| WPRE | forward | 4615 | 5204 |
| rcHS4 Ins-650 insulator | forward | 5340 | 5998 |
| rabbit beta-globin | forward | 6122 | 6570 |
| polyadenylation signal | | | |

In some embodiments, the polynucleotide has 90% identity to that of SEQ ID NO: 55. In some embodiments, the polynucleotide has 95% identity to that of SEQ ID NO: 55. In some embodiments, the polynucleotide has 96% identity to that of SEQ ID NO: 55. In some embodiments, the polynucleotide has 97% identity to that of SEQ ID NO: 55. In some embodiments, the polynucleotide has 98% identity to that of SEQ ID NO: 55. In some embodiments, the polynucleotide has 99% identity to that of SEQ ID NO: 55. In some embodiments, the polynucleotide comprises the nucleic acid sequence of SEQ ID NO: 55 (see also FIG. 1K). In some embodiments, the polynucleotide of SEQ ID NO: 55 comprises the components identified in Table 7:

TABLE 7
Elements of the vector
pTL20c_MND_coWAS_SK734fwd_650
(“pBRNGTR25”).

| | | Start | End |
|---|---|---|---|
| Element | Orientation | (nt position) | (nt position) |
| 7tetO promoter | forward | 28 | 315 |
| MND promoter | forward | 2402 | 2748 |
| WAS cDNA | forward | 2790 | 4298 |
| (codon optimized ORF) | | | |
| WPRE | forward | 4307 | 4896 |
| 7SK promoter | forward | 4909 | 5157 |
| shRNA 734 | forward | 5158 | 5204 |
| rcHS4 Ins-650 insulator | forward | 5340 | 5998 |
| rabbit beta-globin | forward | 6122 | 6570 |
| polyadenylation signal | | | |

In some embodiments, the polynucleotide has 90% identity to that of SEQ ID NO: 56. In some embodiments, the polynucleotide has 95% identity to that of SEQ ID NO: 56. In some embodiments, the polynucleotide has 96% identity to that of SEQ ID NO: 56. In some embodiments, the polynucleotide has 97% identity to that of SEQ ID NO: 56. In some embodiments, the polynucleotide has 98% identity to that of SEQ ID NO: 56. In some embodiments, the polynucleotide has 99% identity to that of SEQ ID NO: 56. In some embodiments, the polynucleotide comprises the nucleic acid sequence of SEQ ID NO: 56 (see also FIG. 1L). In some embodiments, the polynucleotide of SEQ ID NO: 56 comprises the components identified in Table 8:

TABLE 8
Elements of the vector
pTL20c_SK734rev_MND_coWAS_650
(“pBRNGTR26”).

| | | Start | End |
|---|---|---|---|
| Element | Orientation | (nt position) | (nt position) |
| 7tetO promoter | forward | 28 | 315 |
| shRNA 734 | reverse | 2402 | 2448 |
| 7SK promoter | reverse | 2449 | 2697 |
| MND promoter | forward | 2710 | 3056 |
| WAS cDNA | forward | 3098 | 4606 |
| (codon optimized ORF) | | | |
| WPRE | forward | 4615 | 5204 |
| rcHS4 Ins-650 insulator | forward | 5340 | 5998 |
| rabbit beta-globin | forward | 6122 | 6570 |
| polyadenylation signal | | | |

In some embodiments, the polynucleotide has 90% identity to that of SEQ ID NO: 57. In some embodiments, the polynucleotide has 95% identity to that of SEQ ID NO: 57. In some embodiments, the polynucleotide has 96% identity to that of SEQ ID NO: 57. In some embodiments, the polynucleotide has 97% identity to that of SEQ ID NO: 57. In some embodiments, the polynucleotide has 98% identity to that of SEQ ID NO: 57. In some embodiments, the polynucleotide has 99% identity to that of SEQ ID NO: 57. In some embodiments, the polynucleotide comprises the nucleic acid sequence of SEQ ID NO: 57 (see also FIG. 1M). In some embodiments, the polynucleotide of SEQ ID NO: 57 comprises the components identified in Table 9:

TABLE 9
Elements of the vector
pTL20c_MND_coWAS_SK734rev_650
(“pBRNGTR27”).

| | | Start | End |
|---|---|---|---|
| Element | Orientation | (nt position) | (nt position) |
| 7tetO promoter | forward | 28 | 315 |
| MND promoter | forward | 2402 | 2748 |
| WAS cDNA | forward | 2790 | 4298 |
| (codon optimized ORF) | | | |
| WPRE | forward | 4307 | 4896 |
| shRNA 734 | reverse | 4915 | 4961 |
| 7SK promoter | reverse | 4962 | 5210 |
| rcHS4 Ins-650 insulator | forward | 5340 | 5998 |
| rabbit beta-globin | forward | 6122 | 6570 |
| polyadenylation signal | | | |

Host Cells

The present disclosure also provides a host cell comprising the novel expression vectors of the present disclosure. A “host cell” or “target cell” means a cell that is to be transformed using the methods and expression vectors of the present disclosure. In some embodiments, the host cells are mammalian cells in which the expression vector can be expressed. Suitable mammalian host cells include, but are not limited to, human cells, murine cells, non-human primate cells (e.g. rhesus monkey cells), human progenitor cells or stem cells, 293 cells, HeLa cells, D17 cells, MDCK cells, BHK cells, and Cf2Th cells. In certain embodiments, the host cell comprising an expression vector of the disclosure is a hematopoietic cell, such as hematopoietic progenitor/stem cell (e.g. CD34-positive hematopoietic progenitor/stem cell), a monocyte, a macrophage, a peripheral blood mononuclear cell, a CD4+ T lymphocyte, a CD8+ T lymphocyte, or a dendritic cell.

The hematopoietic stem cells (e.g. CD4+ T lymphocytes, CD8+ T lymphocytes, and/or monocyte/macrophages) to be transduced with an expression vector of the disclosure can be allogeneic, autologous, or from a matched sibling. The HSCs are, in some embodiments, CD34-positive and can be isolated from the patient's bone marrow or peripheral blood. The isolated CD34-positive HSCs (and/or other hematopoietic cell described herein) is, in some embodiments, transduced with an expression vector as described herein.

In some embodiments, the host cells or transduced host cells are combined with a pharmaceutically acceptable carrier. In some embodiments, the host cells or transduced host cells are formulated with PLASMA-LYTE A (e.g. a sterile, nonpyrogenic isotonic solution for intravenous administration; where one liter of PLASMA-LYTE A has an ionic concentration of 140 mEq sodium, 5 mEq potassium, 3 mEq magnesium, 98 mEq chloride, 27 mEq acetate, and 23 mEq gluconate). In other embodiments, the host cells or transduced host cells are formulated in a solution of PLASMA-LYTE A, the solution comprising between about 8% and about 10% dimethyl sulfoxide (DMSO). In some embodiments, the less than about 2×10⁷ host cells/transduced host cells are present per mL of a formulation including PLASMA-LYTE A and DMSO.

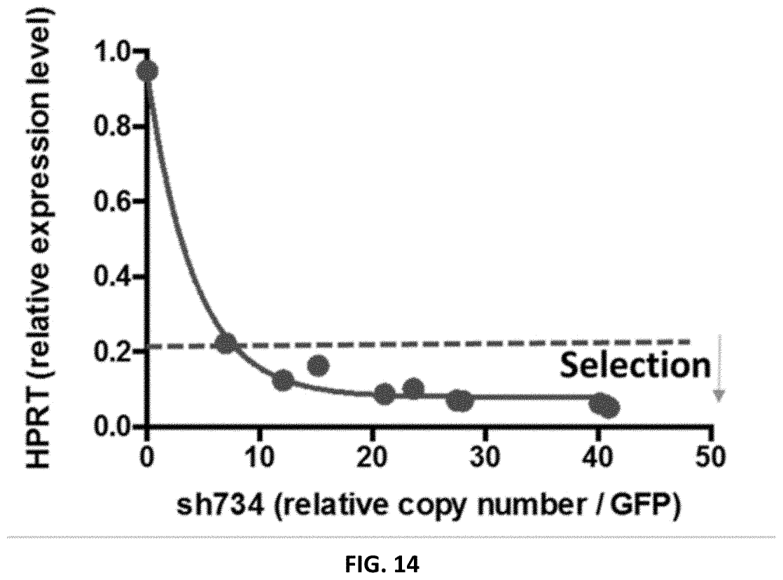

In some embodiments, the host cells are rendered substantially HPRT deficient after transduction with a vector according to the present disclosure. In some embodiments, the level of HPRT gene expression is reduced by at least 50%. In some embodiments, the level of HPRT gene expression is reduced by at least 55%. In some embodiments, the level of HPRT gene expression is reduced by at least 60%. In some embodiments, the level of HPRT gene expression is reduced by at least 65%. In some embodiments, the level of HPRT gene expression is reduced by at least 70%. In some embodiments, the level of HPRT gene expression is reduced by at least 75%. In some embodiments, the level of HPRT gene expression is reduced by at least 80%. In some embodiments, the level of HPRT gene expression is reduced by at least 85%. In some embodiments, the level of HPRT gene expression is reduced by at least 90%. In some embodiments, the level of HPRT gene expression is reduced by at least 95%. It is believed that cells having 20% or less residual HPRT gene expression (e.g. are sensitive to a purine analog, such as 6TG, allowing for their selection with the purine analog (see, for example, FIG. 14). In some embodiments, the host cells include a nucleic acid molecule including at least one of SEQ ID NO: 16 or SEQ ID NO: 17.

In other embodiments, host cells may be transduced with an expression vector comprising (i) a first expression control sequence operably linked to a first nucleic acid sequence, the first nucleic acid sequence encoding a shRNA to knockdown HPRT; and (ii) a second expression control sequence operably linked to a second nucleic acid sequence, the second nucleic acid sequence encoding a Wiskott-Aldrich Syndrome protein. In some embodiments, the transduced host cells are rendered substantially HPRT deficient. In some embodiments, the transduced host cells expresses a Wiskott-Aldrich Syndrome protein.

In some embodiments, transduction of host cells may be increased by contacting the host cell, in vitro, ex vivo, or in vivo, with an expression vector of the present disclosure and one or more compounds that increase transduction efficiency. For example, in some embodiments, the one or more compounds that increase transduction efficiency are compounds that stimulate the prostaglandin EP receptor signaling pathway, i.e. one or more compounds that increase the cell signaling activity downstream of a prostaglandin EP receptor in the cell contacted with the one or more compounds compared to the cell signaling activity downstream of the prostaglandin EP receptor in the absence of the one or more compounds. In some embodiments, the one or more compounds that increase transduction efficiency are a prostaglandin EP receptor ligand including, but not limited to, prostaglandin E2 (PGE2), or an analog or derivative thereof. In other embodiments, the one or more compounds that increase transduction efficiency include but are not limited to, RetroNectin (a 63 kD fragment of recombinant human fibronectin fragment, available from Takara); Lentiboost (a membrane-sealing poloxamer, available from Sirion Biotech), Protamine Sulphate, Cyclosporin H, and Rapamycin.

Pharmaceutical Compositions

The present disclosure also provides for compositions, including pharmaceutical compositions, comprising one or more expression vectors and/or non-viral delivery vehicles (e.g. nanocapsules) as disclosed herein. In some embodiments, pharmaceutical compositions comprise an effective amount of at least one of the expression vectors and/or non-viral delivery vehicles as described herein and a pharmaceutically acceptable carrier. For instance, in certain embodiments, the pharmaceutical composition comprises an effective amount of an expression vector and a pharmaceutically acceptable carrier. An effective amount can be readily determined by those skilled in the art based on factors such as body size, body weight, age, health, sex of the subject, ethnicity, and viral titers.

The phrases “pharmaceutically acceptable” or “pharmacologically acceptable” refer to molecular entities and compositions that do not produce adverse, allergic, or other untoward reactions when administered to an animal or a human. For example, an expression vector may be formulated with a pharmaceutically acceptable carrier. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. Methods for the formulation of compounds with pharmaceutical carriers are known in the art and are described in, for example, in Remington's Pharmaceutical Science, (17th ed. Mack Publishing Company, Easton, Pa. 1985); and Goodman & Gillman's: The Pharmacological Basis of Therapeutics (11th Edition, McGraw-Hill Professional, 2005); the disclosures of each of which are hereby incorporated herein by reference in their entirety.

In some embodiments, the pharmaceutical compositions may comprise any of the expression vectors, nanocapsules, or compositions disclosed herein in any concentration that allows the silencing nucleic acid administered to achieve a concentration in the range of from about 0.1 mg/kg to about 1 mg/kg. In some embodiments, the pharmaceutical compositions may comprise the expression vector in an amount of from about 0.1% to about 99.9% by weight. Pharmaceutically acceptable carriers suitable for inclusion within any pharmaceutical composition include water, buffered water, saline solutions such as, for example, normal saline or balanced saline solutions such as Hank's or Earle's balanced solutions), glycine, hyaluronic acid etc. The pharmaceutical composition may be formulated for parenteral administration, such as intravenous, intramuscular or subcutaneous administration. Pharmaceutical compositions for parenteral administration may comprise pharmaceutically acceptable sterile aqueous or non-aqueous solutions, dispersions, suspensions or emulsions as well as sterile powders for reconstitution into sterile injectable solutions or dispersions. Examples of suitable aqueous and non-aqueous carriers, solvents, diluents or vehicles include water, ethanol, polyols (such as glycerol, propylene glycol, polyethylene glycol, etc.), carboxymethylcellulose and mixtures thereof, vegetable oils (such as olive oil), injectable organic esters (e.g. ethyl oleate).

The pharmaceutical composition may be formulated for oral administration. Solid dosage forms for oral administration may include, for example, tablets, dragees, capsules, pills, and granules. In such solid dosage forms, the composition may comprise at least one pharmaceutically acceptable carrier such as sodium citrate and/or dicalcium phosphate and/or fillers or extenders such as starches, lactose, sucrose, glucose, mannitol, and silicic acid; binders such as carboxylmethylcellulose, alginates, gelatin, polyvinylpyrrolidone, sucrose and acacia; humectants such as glycerol; disintegrating agents such as agar-agar, calcium carbonate, potato or tapioca starch, alginic acid, silicates, and sodium carbonate; wetting agents such as acetyl alcohol, glycerol monostearate; absorbants such as kaolin and bentonite clay; and/or lubricants such as talc, calcium stearate, magnesium stearate, solid polyethylene glycol, sodium lauryl sulfate, and mixtures thereof. Liquid dosage forms for oral administration may include, for example, pharmaceutically acceptable emulsions, solutions, suspensions, syrups and elixirs. Liquid dosages may include inert diluents such as water or other solvents, solubilizing agents and/or emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (such as, for example, cottonseed oil, corn oil, germ oil, castor oil, olive oil, sesame oil), glycerol, tetrahydrofurfuryl alcohol, polyethylene glycols and fatty acid esters of sorbitan, and mixtures thereof.

The pharmaceutical compositions may comprise penetration enhancers to enhance their delivery. Penetration enhancers may include fatty acids such as oleic acid, lauric acid, capric acid, myristic acid, palmitic acid, stearic acid, linoleic acid, linolenic acid, dicaprate, reclineate, monoolein, dilaurin, caprylic acid, arachidonic acid, glyceryl 1-monocaprate, mono and di-glycerides and physiologically acceptable salts thereof. The compositions may further include chelating agents such as, for example, ethylenediaminetetraacetic acid (EDTA), citric acid, salicylates (e.g. sodium salicylate, 5-methoxysalicylate, homovanilate).

The pharmaceutical compositions may comprise any of the expression vectors disclosed herein in an encapsulated form. For example, the expression vectors may be encapsulated within a nanocapsule, such as a nanocapsule comprising one or more biodegradable polymers such as polylactide-polyglycolide, poly(orthoesters) and poly(anhydrides). In some embodiments, the vectors are encapsulated within polymeric nanocapsules. In other embodiments, the vectors are encapsulated within biodegradable and/or erodible polymeric nanocapsules. In some embodiments, the polymeric nanocapsules are comprised of two different positively charged monomers, at least one neutral monomer, and a crosslinker. In some embodiments, the nanocapsules further comprise at least one targeting moiety. In some embodiments, the nanocapsules comprise between 2 and between 6 targeting moieties. In some embodiments, the taretinc moieties are antibodies. In some embodiments, the targeting moieties target any one of the CD117, CD10, CD34, CD38, CD45, CD123, CD127, CD135, CD44, CD47, CD96, CD2, CD4, CD3, and CD9 markers. In some embodiments, the targeting moiety targets any one of a human mesenchymal stem cell CD marker, including the CD29, CD44, CD90, CD49a-f, CD51, CD73 (SH3), CD105 (SH2), CD106, CD166, and Stro-1 markers. In some embodiments, the targeting moiety targets any one of a human hematopoietic stem cell CD marker including CD34, CD38, CD45RA, CD90, and CD49.

In other embodiments, the expression vectors may be encapsulated in liposomes or dispersed within a microemulsion. Liposomes may be, for example, lipofectin or lipofectamine. In another example, a composition may comprise the expression vectors disclosed herein in or on anucleated bacterial minicells (Giacalone et al, Cell Microbiology 2006, 8(10): 1624-33). The expression vectors disclosed herein may be combined with nanoparticles.

Kits

In some embodiments is a kit comprising an expression vector or a composition comprising an expression vector as described herein. The kit may include a container, where the container may be a bottle comprising the expression vector or composition in an oral or parenteral dosage form, each dosage form comprising a unit dose of the expression vector. The kit may comprise a label or the like, indicating treatment of a subject according to the methods described herein.

In some embodiments, the kit may include additional active agents. The additional active agents may be housed in a container separate from the container housing the vector or composition comprising the vector. For example, in some embodiments, the kit may comprise one or more doses of a purine analog (e.g. 6TG) and optionally instructions for dosing the purine analog for conditioning and/or chemoselection (as those steps are described further herein). In other embodiments, the kit may comprise one or more doses of a dihydrofolate reductase inhibitor (e.g. MTX or MPA) and optionally instructions for dosing the dihydrofolate reductase inhibitor for negative selection as described herein.

Methods of Treatment

By way of example, an expression vector including a nucleic acid sequence encoding a WAS gene may be administered so as to genetically correct Wiskott-Aldrich Syndrome or to alleviate the pathologies associated with Wiskott-Aldrich Syndrome. In some embodiments, a population of host cells transduced with an expression vector including a nucleic acid sequence encoding a WAS gene may be administered so as to correct Wiskott-Aldrich Syndrome or to alleviate the pathologies associated with Wiskott-Aldrich Syndrome. It is believed that this method is advantageous over currently available therapies, due to its availability to all patients, particularly those who do not have a matched sibling donor. It is further believed that this method also has the potential to be administered as a one-time treatment providing lifelong correction. It is also believed that the method is advantageously devoid of any immune side effects, and if side effects did arise, the side-effects could be mitigated by administering a dihydrofolate reductase inhibitor (e.g. MTX or MPA) as noted herein. It is further believed that an effective gene therapy approach will revolutionize the way Wiskott-Aldrich Syndrome is treated, ultimately improving patient outcome.

In some embodiments, treatment with the vectors or transduced host cells described herein genetically corrects or alleviates one or more of the pathologies associated with Wiskott-Aldrich Syndrome, such as those outlined below. In some embodiments, the pathologies which may be genetically corrected or alleviated by administering the expression vectors or transduced host cells to a patient include, but are not limited to, microthrombocytopenia, eczema, autoimmune diseases, and recurrent infections. An eczema rash is common in patients with classic WAS. In infants, the eczema may occur on the face or scalp and can resemble “cradle cap.” It can also have the appearance of a severe diaper rash, or be more generalized, involving the arms and legs. In older boys, eczema is often limited to the skin creases around the front of the elbows or behind the knees, behind the ears, or around the wrist. Since eczema is extremely itchy, patients often scratch themselves until they bleed, even while asleep. These areas where the skin barrier is broken can then serve as entry points for bacteria that can cause skin and blood stream infections.

It is believed that thrombocytopenia (a reduced number of platelets) is a common feature of patients with Wiskott-Aldrich Syndrome. In addition to being decreased in number, the platelets themselves are small and dysfunctional, less than half the size of normal platelets. As a result, patients with Wiskott-Aldrich Syndrome may bleed easily, even if they have not had an injury. In some embodiments, bleeding into the skin may cause pinhead sized bluish-red spots, called petechiae, or they may be larger and resemble bruises.

It is believed that the immunodeficiency associated with Wiskott-Aldrich Syndrome causes the function of both B- and T-lymphocytes to be significantly abnormal. As a result, infections are common in the classic form of Wiskott-Aldrich Syndrome and may involve all classes of microorganisms. In some embodiments, these infections may include upper and lower respiratory infections such as ear infections, sinus infections and pneumonia. More severe infections such as sepsis (bloodstream infection or “blood poisoning”), meningitis and severe viral infections are less frequent but can occur. Occasionally, patients with the classic form of Wiskott-Aldrich Syndrome may develop pneumonia caused by the fungus (pneumocystis jiroveci carinii). In some embodiments, the skin may become infected with bacteria such as Staphylococcus in areas where patients have scratched their eczema. In some embodiments, a viral skin infection called molluscum contagiosum is also commonly seen in Wiskott-Aldrich Syndrome. It is believed that vaccination to prevent infections is often not effective in Wiskott-Aldrich Syndrome since patients do not make normal protective antibody responses to vaccines.

In some embodiments, the recurrent infections include, but are not limited to, otitis media, skin abscess, pneumonia, enterocolitis, meningitis, sepsis, and urinary tract infection. In some embodiments, the recurrent infections are cutaneous infections. In some embodiments, the eczema experienced by patients diagnosed with Wiskott-Aldrich Syndrome is classified as treatment-resistant eczema.

By way of example, autoimmune diseases often experienced by those having Wiskott-Aldrich Syndrome include hemolytic anemia, vasculitis, arthritis, neutropenia, inflammatory bowel disease, and IgA nephropathy, Henoch-Schönlein-like purpura, dermatomyositis, recurrent angioedema, and uveitis. In some embodiments, the recurrent infections may be caused by any of a bacterial, viral, or fungal infection. In some embodiments, treatment with the vectors or transduced host cells described herein genetically corrects or alleviates a plurality of the pathologies associated with Wiskott-Aldrich Syndrome, such as those outlined below.

As noted herein, in addition to the therapeutic gene, the expression vectors of the present disclosure include an agent designed to knock down (e.g. a shRNA to HPRT to effect knockdown of HPRT expression), and hence provide for an in vivo chemoselection strategy that exploits the essential role that HPRT plays in metabolizing purine analogs, e.g. 6TG, into myelotoxic agents. Because HPRT-deficiency does not impair hematopoietic cell development or function, it can be removed from hematopoietic cells used for transplantation. Conditioning and chemoselection with a purine analog are discussed further herein.

In the context of the treatment of or alleviation of the pathologies associated with Wiskott-Aldrich Syndrome, and with reference to FIG. 11, the treatment of a subject includes: identifying a subject in need of treatment thereof; transducing HSCs (e.g. autologous HSCs, allogenic HSCs, sibling matched HSCs) with an expression vector (e.g. a lentiviral vector) of the present disclosure (step 120); and transplanting or administering the transduced HSCs into the subject (step 140). In some embodiments, the subject in need of treatment thereof is one suffering from the pathologies associated with Wiskott-Aldrich Syndrome.

In some embodiments, the method of treating hemoglobinopathies comprises (i) transducing HSCs with a vector comprising at least two nucleic acid sequences, namely a nucleic acid sequence encoding a shRNA to knockdown the HPRT gene, and a nucleic acid sequence encoding WASP, and (ii) administering the transduced HSCs to a mammalian subject (e.g. a human patient). In some embodiments, the method further comprises a step of myeloablative conditioning prior to the administration of the transduced HSCs (e.g. using a purine analog, chemotherapy, radiation therapy, treatment with one or more internalizing immunotoxins or antibody-drug conjugates, or any combination thereof). In some embodiments, the method further comprises the step of in vivo chemoselection utilizing a purine analog (e.g. 6TG) following administration of the transduced HSCs. In some embodiments, the method further comprises the step of negative selection utilizing a dihydrofolate reductase inhibitor (e.g. MTX or MPA) should side effects arise (e.g. GvHD).

In another aspect of the present disclosure is a method of alleviating one or more pathologies associated with Wiskott-Aldrich Syndrome, comprising administering an effective amount of a pharmaceutical composition to a mammalian subject (e.g. a human patient), wherein the pharmaceutical compositions include an expression vector comprising at least two nucleic acid sequences, and a pharmaceutically acceptable carrier. In another aspect of the present disclosure is a method of alleviating the pathologies associated with Wiskott-Aldrich Syndrome comprising administering an effective amount of a pharmaceutical composition to a mammalian subject (e.g. a human patient), wherein the pharmaceutical compositions include a population of host cells transduced with an expression vector comprising at least two nucleic acid sequences, and a pharmaceutically acceptable carrier. In some embodiments, the expression vector is a lentiviral expression vector including a first nucleic acid encoding an RNAi to knockdown the HPRT gene; and a second nucleic acid encoding WASP. In some embodiments, the method further comprises a step of myeloablative conditioning prior to the administration of the transduced HSCs. In some embodiments, the method further comprises the step of in vivo chemoselection utilizing 6TG following administration of the transduced HSCs. In some embodiments, the method further comprises the step of negative selection utilizing a dihydrofolate reductase inhibitor (e.g. MTX or MPA) should side effects arise (e.g. GvHD).

Conditioning and Chemoselection with a Purine Analog

In some embodiments, the method of treatment comprises the additional steps of (i) conditioning prior to HSC transplantation; and/or (ii) in vivo chemoselection. One or both steps may utilize a purine analog. In some embodiments, the purine analog is 6TG. It is believed that the engrafted Wiskott-Aldrich Syndrome protein-containing HSCs deficient in HPRT activity are highly resistant to the cytotoxic effects of the introduced purine analog. With a combined strategy of conditioning and chemoselection, efficient and high engraftment of HPRT-deficient, Wiskott-Aldrich Syndrome protein-containing HSCs with low overall toxicity can be achieved. It is believed that resultant expression of the Wiskott-Aldrich Syndrome protein, combined with the enhanced engraftment and chemoselection of gene-modified HSCs, can result in sufficient protein production to alleviate the pathologies associated with Wiskott-Aldrich Syndrome.

6TG is a purine analog having both anticancer and immune-suppressive activities. Thioguanine competes with hypoxanthine and guanine for the enzyme hypoxanthine-guanine phosphoribosyltransferase (HGPRTase) and is itself converted to 6-thioguanylic acid (TGMP). This nucleotide reaches high intracellular concentrations at therapeutic doses. TGMP interferes several points with the synthesis of guanine nucleotides. It inhibits de novo purine biosynthesis by pseudo-feedback inhibition of glutamine-5-phosphoribosylpyrophosphateamidotransferase—the first enzyme unique to the de novo pathway for purine ribonucleotide. TGMP also inhibits the conversion of inosinic acid (IMP) to xanthylic acid (XMP) by competition for the enzyme IMP dehydrogenase. At one-time TGMP was felt to be a significant inhibitor of ATP:GMP phosphotransferase (guanylate kinase), but recent results have shown this not to be so. Thioguanylic acid is further converted to the di- and tri-phosphates, thioguanosine diphosphate (TGDP) and thioguanosine triphosphate (TGTP) (as well as their 2′-deoxyribosyl analogues) by the same enzymes which metabolize guanine nucleotides.

As those of skill in the art will appreciate, given the inclusion of an agent designed to reduce HPRT expression, e.g. an RNAi agent to knockdown HPRT, in the vectors of the present disclosure, the resulting transduced HSCs are HPRT-deficient or substantially HPRT-deficient (e.g. such as those having 20% or less residual HPRT gene expression). As such, those HSCs that do express HPRT, i.e. HPRT wild-type cells, may be selectively depleted by administering one or more doses of 6TG. In some embodiments, 6TG may be administered for both myeloablative conditioning of HPRT-wild type recipients and for in vivo chemoselection process of donor cells. Hence, this strategy is believed to allow for the selection of gene-modified cells in vivo, i.e. for the selection of the Wiskott-Aldrich Syndrome protein-containing gene-modified cells in vivo.

With reference again to FIG. 11, in some embodiments, following the collection of HSCs from a donor (step 110), the HSCs are transduced with a vector according to the present disclosure (step 120). The resulting HSCs are HPRT-deficient and express the WAS gene. In parallel, a patient to receive the HSCs is first treated with a myeloablative conditioning step (step 130). Following conditioning, the transduced HSCs are transplanted or administered to the patient (step 140). The WAS gene containing HSCs may then be selected for (step 150) in vivo using 6TG, as discussed herein.

Myeloablative conditioning may be achieved using high-dose conditioning radiation, chemotherapy, and/or treatment with a purine analog (e.g. 6TG). In some embodiments, the HSCs are administered between about 24 and about 96 hours following treatment with the conditioning regimen. In other embodiments, the patient is treated with the HSC graft between about 24 and about 72 hours following treatment with the conditioning regimen. In yet other embodiments, the patient is treated with the HSC graft between about 24 and about 48 hours following treatment with the conditioning regimen. In some embodiments, the HSC graft comprises between about 2×10⁶ cells/kg to about 15×10⁶ cells/kg (body weight of patient). In some embodiments, the HSC graft comprises a minimum of 2×10⁶ cells/kg, with a target of greater than 6×10⁶ cells/kg. In some embodiments, at least 10% of the cells administered are transduced with a lentiviral vector as described herein. In some embodiments, at least 20% of the cells administered are transduced with a lentiviral vector as described herein. In some embodiments, at least 30% of the cells administered are transduced with a lentiviral vector as described herein. In some embodiments, at least 40% of the cells administered are transduced with a lentiviral vector as described herein. In some embodiments, at least 50% of the cells administered are transduced with a lentiviral vector as described herein.

In some embodiments, the therapeutic gene containing, HPRT-deficient HSCs are selected for in vivo using a low dose schedule of 6TG, which is believed to have minimal adverse effects on extra-hematopoietic tissues. In some embodiments, a dosage of 6TG for in vivo chemoselection ranging from between about 0.2 mg/kg/day to about 0.6 mg/kg/day is provided to a patient following introduction of the HSCs into the patient. In some embodiments, the dosage ranges from between about 0.3 mg/kg/day to about 1 mg/kg/day. In some embodiments, the dosage is up to about 2 mg/kg/day.

In some embodiments, the amount of 6TG administered per dose is based on a determination of a patient's HPRT enzyme activity. Those of ordinary skill in the art will appreciate that those presenting with higher levels of HPRT enzyme activity may be provided with doses having lower amounts of 6TG. The higher the level of HPRT the greater conversion of 6TG to toxic metabolites. Therefore, the lower dose you would need to administer to achieve the same goal.

Measurement of TPMT genotypes and/or TPMT enzyme activity before instituting 6TG conditioning may identify individuals with low or absent TPMT enzyme activity. As such, in other embodiments, the amount of 6TG administered is based on thiopurine S-methyltransferase (TPMT) levels or TPMT genotype.

In some embodiments, the dosage of 6TG for in vivo chemoselection is administered to the patient one to three times a week on a schedule with a cycle selected from the group consisting of: (i) weekly; (ii) every other week; (iii) one week of therapy followed by two, three or four weeks off; (iv) two weeks of therapy followed by one, two, three or four weeks off; (v) three weeks of therapy followed by one, two, three, four or five weeks off; (vi) four weeks of therapy followed by one, two, three, four or five weeks off; (vii) five weeks of therapy followed by one, two, three, four or five weeks off; and (viii) monthly.

In some embodiments, between about 3 and about 10 dosages of 6TG are administered to the patient over an administration period ranging from 1 week to about 4 weeks. In some embodiments, 4 or 5 dosages of 6TG are administered to the patient over a 14-day period.

Negative Selection with a Dihydrofolate Reductase Inhibitor

In addition, HPRT-deficient cells can be negatively selected by using a dihydrofolate reductase inhibitor (e.g. MTX) to inhibit the enzyme dihydrofolate reductase (DHFR) in the purine de novo synthetic pathway. This has been developed as a safety procedure to eliminate gene-modified HSCs in case of unexpected adverse effects observed. As such, and in reference to FIG. 11, should any adverse side effects arise, a patient may be treated with a dihydrofolate reductase inhibitor (e.g. MTX or MPA) (step 160). Adverse side effects include, for example, aberrant blood counts/clonal expansion indicating insertional mutagenesis in a particular clone of cells or cytokine storm.

It is believed that a dihydrofolate reductase inhibitor (e.g. MTX or MPA) competitively inhibits dihydrofolate reductase (DHFR), an enzyme that participates in tetrahydrofolate (THF) synthesis. DHFR catalyzes the conversion of dihydrofolate to active tetrahydrofolate. Folic acid is needed for the de novo synthesis of the nucleoside thymidine, required for DNA synthesis. Also, folate is essential for purine and pyrimidine base biosynthesis, so synthesis will be inhibited. The dihydrofolate reductase inhibitor (e.g. MTX or MPA) therefore inhibits the synthesis of DNA, RNA, thymidylates, and proteins. MTX or MPA blocks the de novo pathway by inhibiting DHFR. In HPRT−/− cell, there is no salvage or de novo pathway functional, leading to no purine synthesis, and therefore the cells die. However, the HPRT wild type cells have a functional salvage pathway, their purine synthesis takes place and the cells survive.

Given the sensitivity of the modified HSCs produced according to the present disclosure, a dihydrofolate reductase inhibitor (e.g. MTX or MPA) may be used to selectively eliminate HPRT-deficient cells. In some embodiments, a dihydrofolate reductase inhibitor (e.g. MTX or MPA) is administered as a single dose. In some embodiments, multiple doses of the dihydrofolate reductase inhibitor are administered.

In some embodiments, an amount of MTX administered ranges from about 2 mg/m²/infusion to about 100 mg/m²/infusion. In some embodiments, an amount of MTX administered ranges from about 2 mg/m²/infusion to about 90 mg/m²/infusion. In some embodiments, an amount of MTX administered ranges from about 2 mg/m²/infusion to about 80 mg/m²/infusion. In some embodiments, an amount of MTX administered ranges from about 2 mg/m²/infusion to about 70 mg/m²/infusion. In some embodiments, an amount of MTX administered ranges from about 2 mg/m²/infusion to about 60 mg/m²/infusion. In some embodiments, an amount of MTX administered ranges from about 2 mg/m²/infusion to about 50 mg/m²/infusion. In some embodiments, an amount of MTX administered ranges from about 2 mg/m²/infusion to about 40 mg/m²/infusion. In some embodiments, an amount of MTX administered ranges from about 2 mg/m²/infusion to about 30 mg/m²/infusion. In some embodiments, an amount of MTX administered ranges from about 20 mg/m²/infusion to about 20 mg/m²/infusion. In some embodiments, an amount of MTX administered ranges from about 2 mg/m²/infusion to about 10 mg/m²/infusion. In some embodiments, an amount of MTX administered ranges from about 2 mg/m²/infusion to about 8 mg/m²/infusion. In other embodiments, an amount of MTX administered ranges from about 2.5 mg/m²/infusion to about 7.5 mg/m²/infusion. In yet other embodiments, an amount of MTX administered is about 5 mg/m²/infusion. In yet further embodiments, an amount of MTX administered is about 7.5 mg/m²/infusion.

In some embodiments, between 2 and 6 infusions are made, and the infusions may each comprise the same dosage or different dosages (e.g. escalating dosages, decreasing dosages, etc.). In some embodiments, the administrations may be made on a weekly basis, or a bi-monthly basis.

In some embodiments, MPA is dosed in an amount of between about 500 mg to about 1500 mg per day. In some embodiments, the dose of MPA is administered in a single bolus. In some embodiments, the dose of MPA is divided into a plurality of individual doses totaling between about 500 mg to about 1500 mg per day.

In some embodiments, an analog or derivative of MTX or MPA may be substituted for MTX or MPA. Derivatives of MTX are described in U.S. Pat. No. 5,958,928 and in PCT Publication No. WO/2007/098089, the disclosures of which are hereby incorporated by reference herein in their entireties. In some embodiments, an alternative agent may be used in place of either MTX or MPA, including, but not limited to ribavarin (IMPDH inhibitor); VX-497 (IMPDH inhibitor) (see Jain J, VX-497: a novel, selective IMPDH inhibitor and immunosuppressive agent, J Pharm Sci. 2001 May; 90(5):625-37); lometrexol (DDATHF, LY249543) (GAR and/or AICAR inhibitor); thiophene analog (LY254155) (GAR and/or AICAR inhibitor), furan analog (LY222306) (GAR and/or AICAR inhibitor) (see Habeck et al., A Novel Class of Monoglutamated Antifolates Exhibits Tight-binding Inhibition of Human Glycinamide Ribonucleotide Formyltransferase and Potent Activity against Solid Tumors, Cancer Research 54, 1021-2026, February 1994); DACTHF (GAR and/or AICAR inhibitor) (see Cheng et. al. Design, synthesis, and biological evaluation of 10-methanesulfonyl-DDACTHF, 10-methanesulfonyl-5-DACTHF, and 10-methylthio-DDACTHF as potent inhibitors of GAR Tfase and the de novo purine biosynthetic pathway; Bioorg Med Chem. 2005 May 16; 13(10):3577-85); AG2034 (GAR and/or AICAR inhibitor) (see Boritzki et. al. AG2034: a novel inhibitor of glycinamide ribonucleotide formyltransferase, Invest New Drugs. 1996; 14(3):295-303); LY309887 (GAR and/or AICAR inhibitor) ((2S)-2-[[5-[2-[(6R)-2-amino-4-oxo-5,6,7,8-tetrahydro-1H-pyrido[2,3-d]pyrimidin-6-yl]ethyl]thiophene-2-carbonyl]amino]pentanedioic acid); alimta (LY231514) (GAR and/or AICAR inhibitor) (see Shih et. al. LY231514, a pyrrolo[2,3-d]pyrimidine-based antifolate that inhibits multiple folate-requiring enzymes, Cancer Res. 1997 Mar. 15; 57(6):1116-23); dmAMT (GAR and/or AICAR inhibitor), AG2009 (GAR and/or AICAR inhibitor); forodesine (Immucillin H, BCX-1777; trade names Mundesine and Fodosine) (inhibitor of purine nucleoside phosphorylase [PNP]) (see Kicska et. al., Immucillin H, a powerful transition-state analog inhibitor of purine nucleoside phosphorylase, selectively inhibits human T lymphocytes, PNAS Apr. 10, 2001. 98 (8) 4593-4598); and immucillin-G (inhibitor of purine nucleoside phosphorylase [PNP]).

Combination Therapy

In another aspect of the present disclosure is a combination therapy whereby antibacterial, antifungal, and/or antiviral active pharmaceutical ingredients (depending, of course, upon the particular infection presented) are administered prior to, during, or following the administration or transplantation of transduced HSCs (described above) into a patient in need of treatment thereof, e.g. to alleviate the pathologies associated with Wiskott-Aldrich Syndrome. In some embodiments, patients with Wiskott-Aldrich Syndrome and having severe thrombocytopenia may be treated with high dose intravenous immunoglobulin (2 gm/kg/day) and/or corticosteroids (2 mg/kg/day) prior to, during, or following the administration or transplantation of transduced HSCs (described above) into a patient in need of treatment thereof. Alternatively, an allogenic transplantation of stem cells from healthy donors may be administered before or after treatment with the expression vectors or transduced stem cells of the present disclosure.

### EXAMPLES

### Example 1: Intermediate Vector Production—pTL20c Vectors Comprising 400 bp Insulator

TABLE 10
Four intermediate vectors incorporating a wild type (wt) or codon optimized
(co) WAS transgene, shRNA, cHS4 400 bp insulator, MND promoter.

| No. | Name | SEQ ID NO: |
|---|---|---|
| Int 1 | pTL20c_7SK/sh734_MND/hWAS^{WT}_WPRE_7SK/sh734_Ins400 | 63 |
| Int 2 | pTL20c_r7SK/sh734_MND_hWAS^{CO}_WPRE_r7SK/sh734_Ins400 | 64 |
| Int 3 | pTL20c_r7SK/sh734_MND/hWAS^{WT}_WPRE_r7SK/sh734_Ins400 | 65 |
| Int 4 | pTL20c_7SK/sh734_MND/hWAS^{CO}_WPRE_7SK/sh734_Ins400 | 66 |

“Int 1” vector (Table 10) was generated by inserting a 3.2 kb hWASWT cassette (SEQ ID NO: 58) into BstBI and NotI sites of pTL20c vector. The 3.2 kb was synthesized and contains (in the following order): 1) the 7SK promoter and short hairpin RNA (shRNA) 734 expression cassette (SEQ ID NO: 14) in forward orientation; 2) WAS expression cassette consisting of MND promoter, hWASWT cDNA (SEQ ID NO: 1) and WPRE element (SEQ ID NO: 13); and 3) the 7SK promoter and short hairpin RNA (shRNA) 734 expression cassette (SEQ ID NO: 14) in forward orientation. The WASWT cDNA contains two silent mutations to remove two internal SfiI sites.

“Int 2” vector (Table 10) was generated by inserting a 3.2 kb hWAS^{CO} cassette (SED ID: 59) into BstBI and NotI sites of pTL20c vector. The 3.2 kb was synthesized and contains (in the following order):1) the 7SK promoter and short hairpin RNA (shRNA) 734 expression cassette (SEQ ID NO: 14) in reverse orientation; 2) WAS expression cassette consisting of MND promoter, hWAS^{CO} cDNA (SEQ ID NO: 4) and WPRE element (SEQ ID NO: 13); and 3) the 7SK promoter and short hairpin RNA (shRNA) 734 expression cassette (SEQ aID NO: 14) in reverse orientation. The hWAS^{CO} cDNA was codon-optimized.

“Int 3” vector (Table 10) which contains two shRNA expression cassettes in the reverse orientation, was generated by inserting the hWASWT cDNA into AscI/SpeI sites of the pTL20c_r7SK/sh734_MND_hWASm_WPRE_r7SK/sh734_Ins400 vector (SEQ ID No: 64), replacing the hWAS^{CO} cDNA.

“Int 4” vector (Table 10), which contains two shRNA expression cassettes in the forward orientation, was generated by inserting the hWAS^{CO} cDNA into AscI/SpeI sites of the pTL20c_7SK/sh734_MND_hWASwT_WPRE_7SK/sh734_Ins400 vector (SEQ ID NO: 63), replacing the hWASWT cDNA.

### Example 2: Vector Production—pTL20c Vectors Comprising 400 bp Insulator

Vector candidates comprising a 400 bp extended core element of the chicken hypersensitivity site 4 insulator (cHS4) were prepared as set forth in Table 11.

TABLE 11
Eight candidates incorporating wild type (wt) or codon optimized
(co) WAS transgene, shRNA, cHS4 400 bp insulator, MND promoter.

| Candidate No. | Name | SEQ ID NO |
|---|---|---|
| 1 | pTL20c-7SK/sh734-MND/hWAS^{wt} | 42 |
| 2 | pTL20c-MND/hWAS^{wt}-7SK/sh734 | 43 |
| 3 | pTL20c-r7SK/sh734-MND/hWAS^{wt} | 44 |
| 4 | pTL20c-MND/hWAS^{wt}-r7SK/sh734 | 45 |
| 5 | pTL20c-r7SK/sh734-MND/hWAS^{co} | 46 |
| 6 | pTL20c-MND/hWAS^{co}-r7SK/sh734 | 47 |
| 7 | pTL20c-7SK/sh734-MND/hWAS^{co} | 48 |
| 8 | pTL20c-MND/hWAS^{co}-7SK/sh734 | 49 |

Candidate vectors were prepared from intermediate constructs (Table 10) by removal of one of the short hairpin RNA (shRNA) 734 sequences.

The pTL20c-7SK/sh734-MND/hWASwt vector (SEQ ID NO: 42) was generated by removing the second 7SK/sh734 shRNA expression cassette located downstream of hWAS expression cassette from the “Int 1” vector via AgeI digestion followed by re-ligation.

The pTL20c-MND/hWASwt-7SK/sh734 vector (SEQ ID NO: 43) was generated by removing the second 7SK/sh734 shRNA expression cassette located upstream of hWAS expression cassette from the “Int 1” vector via MluI digestion followed by re-ligation.

The pTL20c-r7SK/sh734-MND/hWASwt vector (SEQ ID NO: 44) was generated by removing the second r7SK/sh734 shRNA expression cassette located downstream of hWAS expression cassette from the “Int 3” vector via AgeI digestion followed by re-ligation.

The pTL20c-MND/hWASwt-r7SK/sh734 vector (SEQ ID NO: 45) was generated by removing the second r7SK/sh734 shRNA expression cassette located upstream of hWAS expression cassette from the “Int 3” vector via MluI digestion followed by re-ligation.

The pTL20c-r7SK/sh734-MND/hWAS^{CO} vector (SEQ ID NO: 46) was generated by removing the second 7SK/sh734 shRNA expression cassette located downstream of hWAS expression cassette from the “Int 4” vector via AgeI digestion followed by re-ligation.

The pTL20c-MND/hWAS^{co}-r7SK/sh734 vector (SEQ ID NO: 47) was generated by removing the r7SK/sh734 shRNA expression cassette located upstream of hWAS expression cassette from the “Int 4” vector via MluI digestion followed by re-ligation.

The pTL20c-7SK/sh734-MND/hWAS^{CO} vector (SEQ ID NO: 48) was generated by removing the second r7SK/sh734 shRNA expression cassette located downstream of hWAS expression cassette from the “Int 2” vector via AgeI digestion followed by re-ligation.

The pTL20c-MND/hWAS^{co}-7SK/sh734 (SEQ ID NO: 49) was generated by removing the second r7SK/sh734 shRNA expression cassette located upstream of hWAS expression cassette from the “Int 2” vector via MluI digestion followed by re-ligation.

### Example 3: Vector Production—pTL20c Vectors Comprising 650 bp Insulator

Candidate vectors comprising a 650 bp extended core element of the chicken hypersensitivity site 4 insulator (cHS4) were prepared according to the protocol which follows.

TABLE 12
Eight candidates incorporating wild type (wt) or codon optimized
(co) WAS transgene, shRNA, cHS4 650 bp insulator, MND promoter.

| Candidate No. | Name | SEQ ID NO. |
|---|---|---|
| 9 | pTL20c_SK734Fwd_pMND_WASwt_Ins650 | 50 |
| 10 | pTL20c_pMND_WASwt_SK734Fwd_Ins650 | 51 |
| 11 | pTL20c_SK734Rev_pMND_WASwt_Ins650 | 52 |
| 12 | pTL20c_pMND_WASwt_SK734Rev_Ins650 | 53 |
| 13 | pTL20c_SK734Fwd_pMND_WASco_Ins650 | 54 |
| 14 | pTL20c_pMND_WASco_SK734Fwd_Ins650 | 55 |
| 15 | pTL20c_SK734Rev_pMND_WASco_Ins650 | 56 |
| 16 | pTL20c_pMND_WASco_SK734Rev_Ins650 | 57 |

Method

pTL20c vector constructs comprising a 650 bp extended core element of the chicken hypersensitivity site 4 insulator (cHS4) sequence (Table 12; Candidate Nos. 9 to 16) were generated. The 650 bp cHS4 sequence was inserted in the 3′ LTR in reverse orientation to the viral transcript. These vectors were prepared by exchanging the 400 bp insulator from preceding vectors Table 11; Candidate Nos. 1-8.

pTL20c vector constructs listed in Table 11, Candidate Nos. 1-8, were digested with NotI and NheI restriction enzymes to remove the 400 bp cHS4 insulator sequence and the digested vector backbones were gel-purified.

Plasmids 11, 12, 15 and 16 (Table 12) were constructed through Gibson Assembly of four fragments. Adjacent to the NotI/NheI-digested vector backbone, each assembly reaction contained a PCR product of the 650 bp cHS4 sequence and two IDT gBlocks Gene Fragments (gBlocks). The gBlocks reintroduced the sequences flanking the insulator in the pTL20c constructs. The sequence between the insulator and the NheI site was reintroduced using gBlock 1 (SEQ ID NO: 60) (Table 13), common to each Gibson Assembly reaction. The sequence between the NotI site and the insulator was reintroduced using gBlock 2 (SEQ ID NO: 61) (Table 12, Candidate Nos. 11 and 15) or gBlock 3 (SEQ ID NO: 62) (Table 12, Candidate Nos. 12 and 16), depending on the absence or presence of a 7sk/sh734 expression cassette in the downstream position, respectively.

TABLE 13
gBlock Sequences.

| | | | SEQ |
|---|---|---|---|
| | Nucleotide | Length/ | ID |
| Name | Sequence (5′ to 3′) | bp | NO: |
| gBlock 1 | GCTGTCCCCGTGAGCTCCCCAGATCTG | 179 | 60 |
| | CTTTTTGCCTGTACTGGGTCTCTCTGG | | |
| | TTAGACCAGATCTGAGCCTGGGAGCTC | | |
| | TCTGGCTAACTAGGGAACCCACTGCTT | | |
| | AAGCCTCAATAAAGCTTCAGCTGCTCG | | |
| | AGCTAGCAGATCTTTTTCCCTCTGCCA | | |
| | AAAATTATGGGGACATC | | |
| gBlock 2 | CTTTGGGCCGCCTCCCCGCACGTACGA | 189 | 61 |
| | CCGGTGCGGCCGCATCGATGCCGTAGT | | |
| | ACCTTTAAGACCAATGACTTACAAGGC | | |
| | AGCTGTAGATCTTAGCCACTTTTTAAA | | |
| | AGAAAAGGGGGGACTGGAAGGGCTAAT | | |
| | TCACTCCCAAAGAAGACAAGGCCCCAT | | |
| | CCTCACTGACTCCGTCCTGGAGTTGGA | | |
| gBlock 3 | GCATGCTAAATACTGCACGTCGATACC | 187 | 62 |
| | GGTGCGGCCGCATCGATGCCGTAGTAC | | |
| | CTTTAAGACCAATGACTTACAAGGCAG | | |
| | CTGTAGATCTTAGCCACTTTTTAAAAG | | |
| | AAAAGGGGGGACTGGAAGGGCTAATTC | | |
| | ACTCCCAAAGAAGACAAGGCCCCATCC | | |
| | TCACTGACTCCGTCCTGGAGTTGGA | | |

Plasmids 9, 10, 13 and 14 (Table 12) were constructed using traditional restriction cloning. The 650 bp cHS4 sequence including flanking regions was isolated by restriction digestion of 15 (Table 12) with NheI and NotI followed by gel-purification. The final plasmids were obtained by ligation of this DNA fragment with the NotI/NheI-digested vector backbones.

### Example 4: Transduction

Materials

293T cells and LV-hWASp/sh7 vectors

Methods

293T cells per well were co-incubated with LV-hWASp/sh7 vectors.

### Example 5: Preliminary Screening Data Transduction and WASp Expression (Mean Fluorescence Intensity)

Intracellular expression of human Wiskott-Aldrich syndrome protein (hWASp) was detected via Flow cytometry.

Materials

BD 5A5 anti-hWASp (BD) Abs was used as the primary antibody. An APC-conjugated Goat Anti-Mouse IgG (Thermo Fisher) antibody was used as a secondary antibody to bind to the primary antibody to assist in detection. BD Stain Buffer (BD), BD Cytofix/Cytoperm Kit (BD) and Fc Receptor Blocker (Innovex) were also used (see Table 14).

Flow cytometry was conducted on a MACSQuant Flow cytometer with a 96-plate reader.

TABLE 14
Summary of Antibodies.

| | | | | | | Final | |
|---|---|---|---|---|---|---|---|
| Type | Antibody | Concentration | | Host | Dilution | Concentration | |
| Primary | 5A5 Anti-WASp | 250 | μg/mL | Mouse | 1:25 | 8 | μg/mL |
| Secondary | APC Goat Anti | 1000 | μg/mL | Goat | 1:100 | 10 | μg/mL |
| | Mouse IgG | | | | | | |

Methods

Sample Preparation

Four days post transduction, the transduced cells were analyzed. Medium was removed and the transduced cells were washed with 500 μL of PBS. PBS was discarded and 200 μl of 1×TrypLE Express was added.

Cells were incubated at room temperature until the cells detached. Cells were resuspended in 1 mL of D10 medium, transferred to a 1.5 mL Eppendorf tube and subsequently centrifuged for 3 minutes at 1,200 rpm in the tabletop centrifuge. Supernatant was subsequently removed with a pipette.

Cells were washed by resuspending in 500 μL of PBS, followed by centrifugation for 3 minutes at 1,200 rpm in the tabletop centrifuge. Supernatant was subsequently with a pipette. Washing was repeated, after which cells were resuspended in 200 μL cold BD Stain Buffer and transferred to a 96-well plate (V-bottom wells) (Controls: with and without staining).

FC Blocking

100 μL of cold BD Stain Buffer was added to each well. Samples were subsequently centrifuged for 3 minutes at 2,000 rpm (800×g). Supernatant was removed quickly by flicking the plate and residual liquid was removed by applying paper towel to the plate. 1 drop (around 50 μL) of Fc Receptor Block was added and the cells were subsequently resuspended by agitation using a multichannel pipet.

Samples were incubated for 30 minutes at 4° C. or on ice. Cells were subsequently washed by addition of 200 μL cold BD Stain Buffer to each well followed by centrifugation for 3 min at 2,000 rpm (800×g). Supernatant was again removed quickly by flicking the plate and residual liquid was removed by applying paper towel to the plate. Washing was then repeated.

Fixation/Permeabilization

100 μL Cytofix/Cytoperm Solution was added to each well and the cells were subsequently resuspended by agitation using a multichannel pipet. Samples were incubated for 30 minutes at 4° C. or on ice, and light was excluded.

Master mixes for the primary antibodies were prepared as noted above. 1×BD Perm/Wash Buffer was prepared comprising 5 mL 10×BD Perm/Wash Buffer+45 mL water and stored at 4° C. Samples were washed repeatedly with cold 1×BD Perm/Wash buffer.

Antibodies Staining

Cells were resuspended in 50 μL 1×BD Perm/Wash buffer containing the primary antibodies and subsequently incubate for 20 minutes at 4° C. with exclusion of light. Samples were repeatedly washed with 1×BD Perm/Wash buffer for 3 minutes at 2,000 rpm (800×g) before being resuspended in 50 μL 1×BD Perm/Wash buffer containing secondary antibodies. Samples were subsequently incubated for 20 minutes at 4° C. with exclusion of light before being repeatedly washed with 1×BD Perm/Wash buffer. Finally, samples were washed with BD Stain Buffer before being resuspended in 200 μL BD Stain Buffer for analysis.

Flow Cytometry

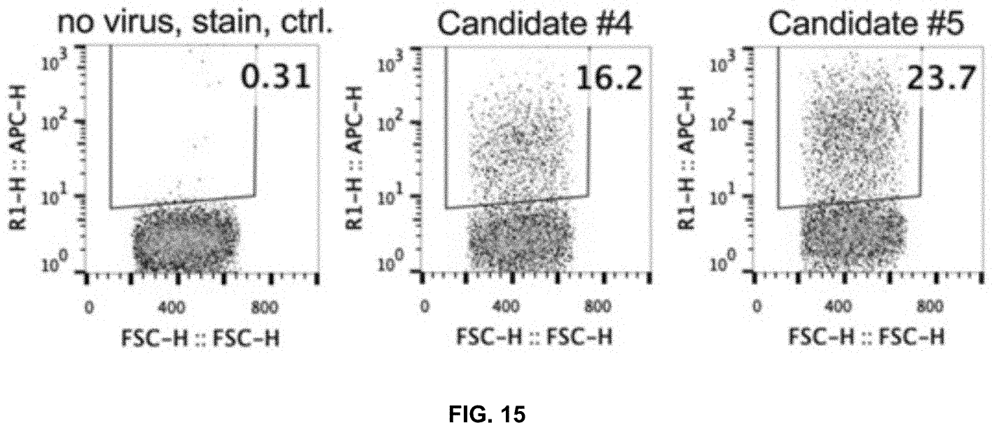

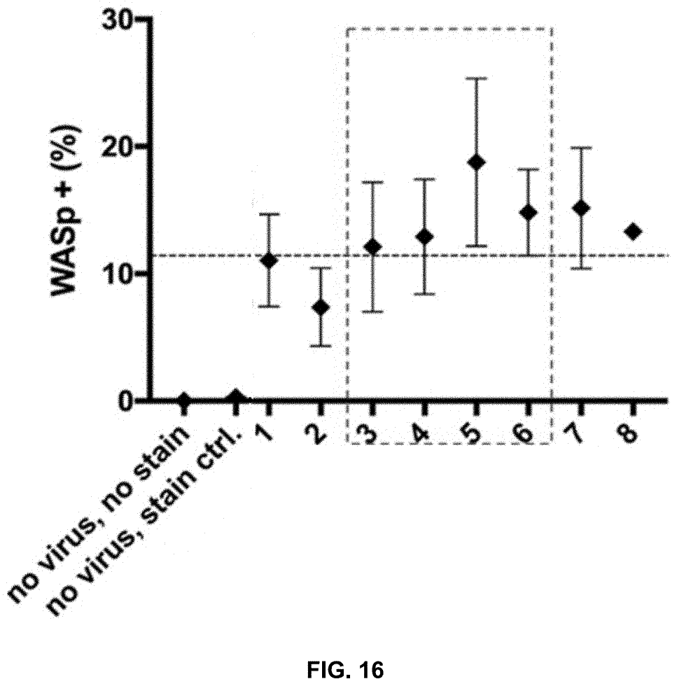

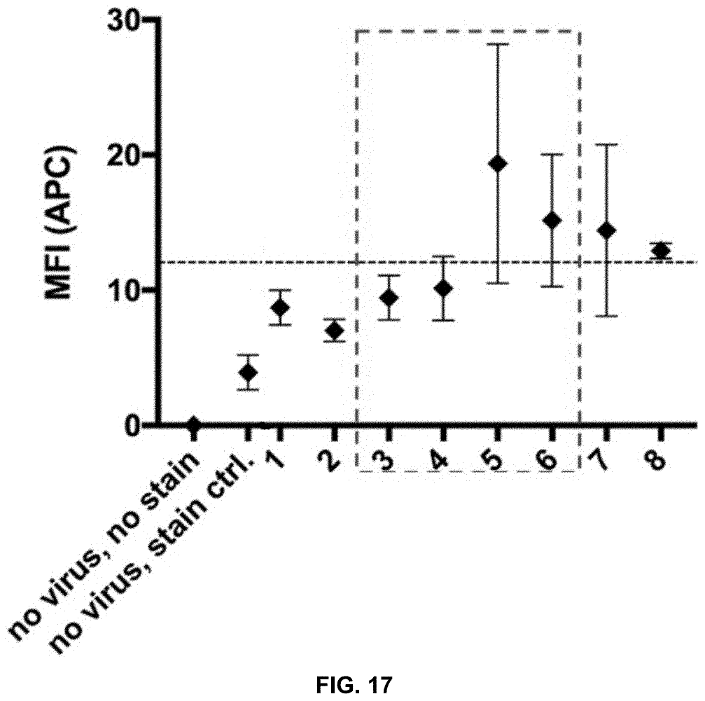

Flow cytometry was conducted on a MACSQuant Flow cytometer fitted with the 96-plate reader (see FIGS. 15-17). Stained 293T cells were gated for sizes (SSC-A vs. FSC-A) and singlets (FSC-H vs. FSC-A). The gate around APC+ cells was delineated. Negative control gating was (Mock, without virus treated, with hWASp staining): 0.5% or less.

Candidates 3, 4, 5, and 6 were progressed for further screening, as noted in Table 15.

TABLE 15
Of the eight candidate vectors including the 400
bp insulator element, four candidates (3, 4, 5,
and 6) were progressed for further screening.

| No. | Name | Proceed |
|---|---|---|
| 1 | pTL20c-7SK/sh734-MND/hWAS^{wt} | |
| 2 | pTL20c-MND/hWAS^{wt}-7SK/sh734 | |
| 3 | pTL20c-r7SK/sh734-MND/hWAS^{wt} | yes |
| 4 | pTL20c-MND/hWAS^{wt}-r7SK/sh734 | yes |
| 5 | pTL20c-r7SK/sh734-MND/hWAS^{co} | yes |
| 6 | pTL20c-MND/hWAS^{co}-r7SK/sh734 | yes |
| 7 | pTL20c-7SK/sh734-MND/hWAS^{co} | |
| 8 | pTL20c-MND/hWAS^{co}-7SK/sh734 | |

### Example 6: Infectious Titres—Vector Copy Versus WASp+

(i) Quantification of Integrated Lentiviral Vector Copy Number (VCN) in Transduced Human Cells by U5 Psi/huRPP30 ddPCR

A VCN assay was used to determine the number of integrated lentiviral vector genomes per host cell genome.

The assay utilizes droplet digital PCR (ddPCR) technology and was performed using the Bio-Rad QX200 ddPCR System with Automated Droplet Generator and. Transduced cells were harvested for genomic DNA extraction. Extracted genomic DNA was analyzed by the ddPCR VCN Assay to determine average vector copy number per cell in a multiplex format.

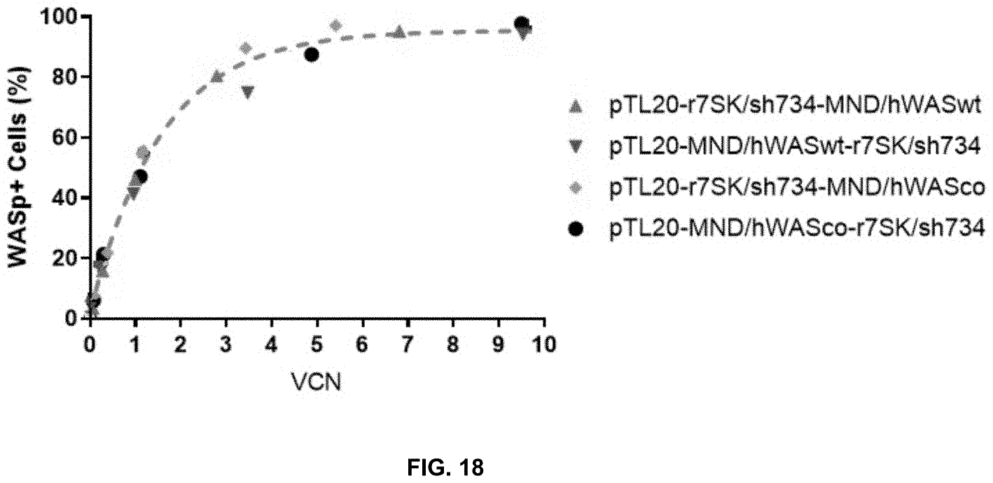

For transduced human cells, the ddPCR VCN assay measured absolute concentrations (copies/μL) of the lentiviral vector target (U5 psi) and a human endogenous reference sequence (huRPP30). VCN is calculated from the ratio of U5 psi to huRPP30 and normalized to the known copy number of huRPP30 in the cell type used (see also FIG. 18).

TABLE 16
Assay Results.

| | | | WASp- | | |
|---|---|---|---|---|---|
| | | | staining-based | VCN-based | |
| | | Select + | Infectious Titer | Infectious Titer | |
| Vector ID | Name | position/location | (×10⁶ TU/mL) | (×10⁶ IU/mL) | SEQ ID NO: |
| — | Negative Ctrl | — | NA | NA | — |
| GFP | TL20cw-r7SK/ | Upstream/reverse | 159.4 | 265.67 | — |
| | sh734-Ubc/GFP | | | | |
| | (concentrated) | | | | |
| 3 | TL20cw-r7SK/ | Upstream/reverse | 4.34 | 7.23 | 43 |
| | sh734-MND/WASp^{wt} | | | | |
| 4 | TL20cw-MND/ | Downstream/reverse | 5.41 | 9.02 | 45 |
| | WASp^{wt}-r7SK/sh734 | | | | |
| 5 | TL20cw-r7SK/ | Upstream/reverse | 7.10 | 11.83 | 47 |
| | sh734-MND/WASp^{co} | | | | |
| 6 | TL20cw-MND/ | Downstream/reverse | 5.90 | 9.83 | 49 |
| | WASp^{co}-r7SK/sh734 | | | | |
| 1 | TL20cw-7SK/ | Upstream/forward | 2.08 | 3.47 | 42 |
| | sh734-MND/WASp^{wt} | | | | |
| 7 | TL20cw-7SK/ | Upstream/forward | 3.04 | 5.07 | 46 |
| | sh734-MND/WASp^{co} | | | | |

(ii) Variation of WASp Expression in MFI (Mean Fluorescence Intensity) Per Vector Copy Number (VCN)

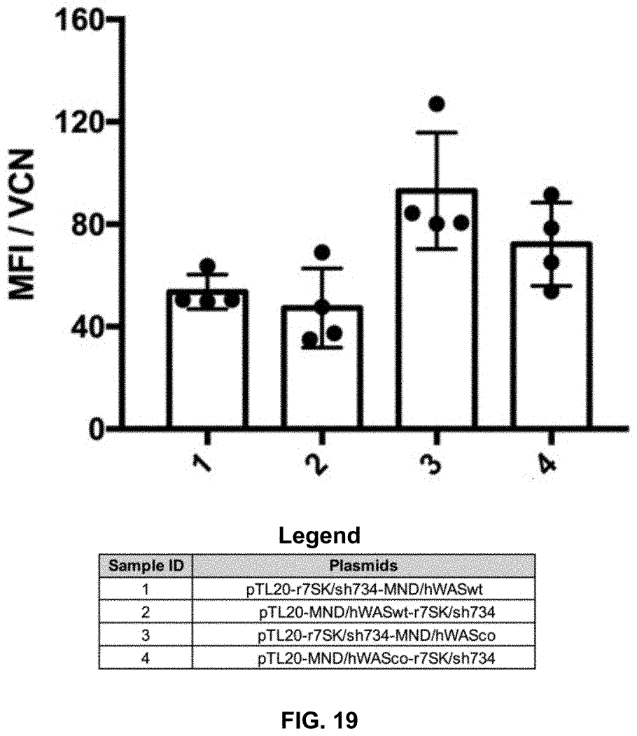

Variation of WASp expression in MFI (Mean Fluorescence Intensity) per Vector Copy Number in transduced 293T cells containing 0.2 to 9 copies of parental control vector and the 4 candidate vectors (n=4) is illustrated in FIG. 19.

(iii) Infectious Titer—LV Candidates Comprising 400-Ins

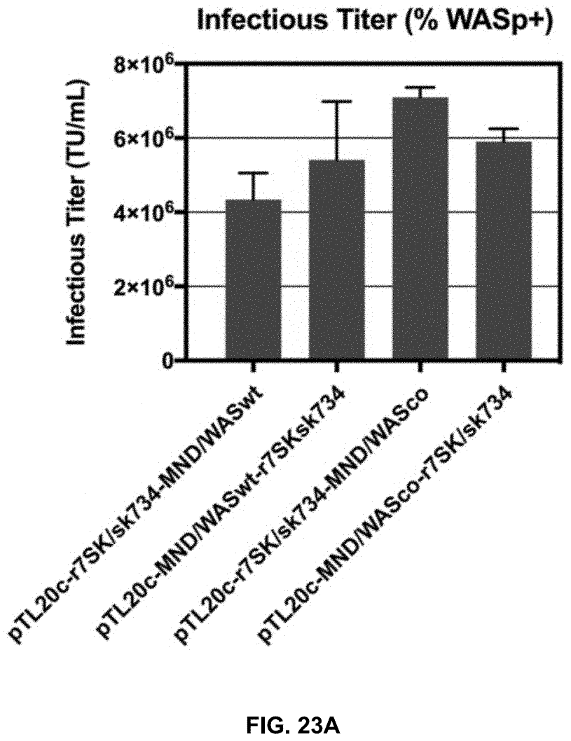

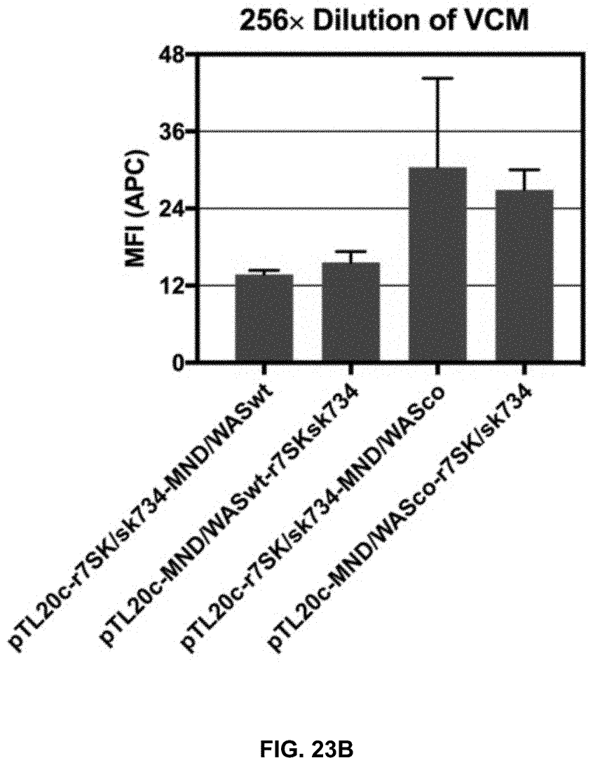

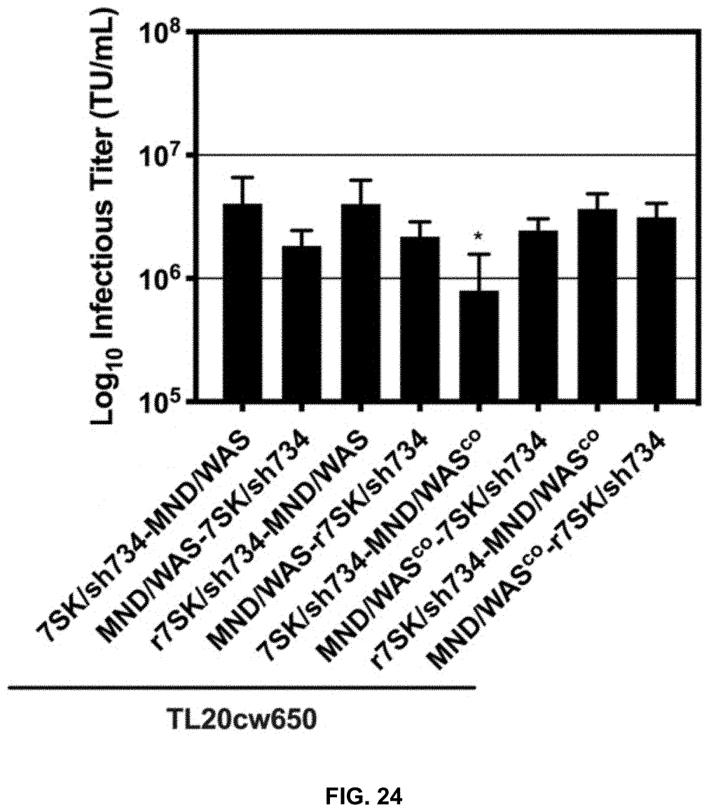

To measure infectious Titer, 293T cells were transduced by co-incubation of lentiviral vector candidates comprising 400-Ins with diluted VCM. The results are illustrated in FIGS. 23A and 23B. Titer levels were also measured for cells transduced with lentiviral vector candidates comprising 650-Ins (see FIG. 24).

### Example 7: 6TG Selection/Resistance Assay

i) Titration 6TG Dosing Window

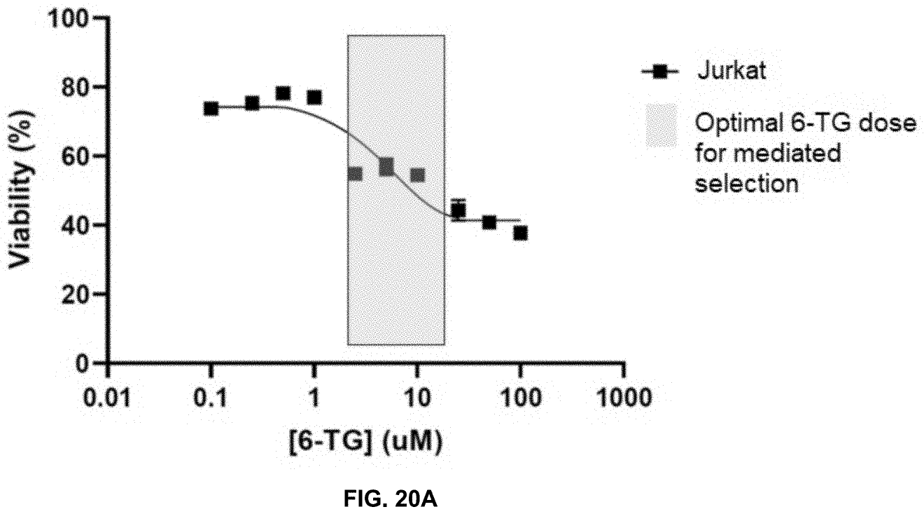

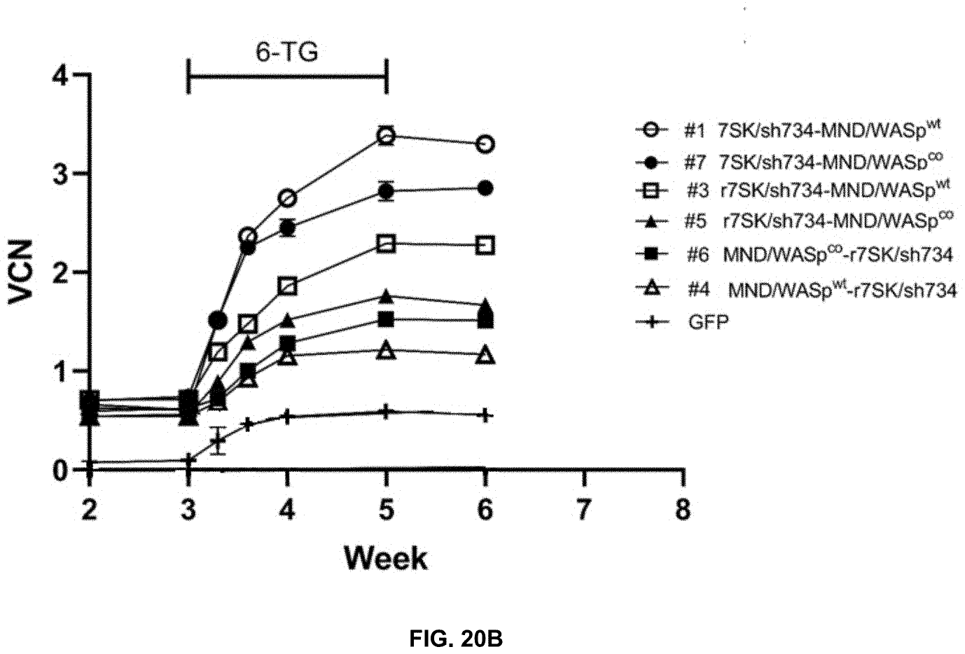

Jurkat cells were incubated in a 96-well plate with different concentrations of 6TG (0.01-100 μM). After 2 days, viability of the cells was measured with cell counter TC20. Optimal 6TG dose for Jurkat cells was estimated to be approx. 504. A nominal concentration of 2.5 μM 6TG was elected for subsequent chemo-selection experiments. The results are illustrated in FIG. 20A.

ii) Chemo-Selection of Transduced Jurkat Cells with 6TG

Jurkat cells were transduced with representative vector candidates at M01=0.5 and culture for 3 weeks before 2-weeks treatment with 2.5 μM 6TG. At week 5, the cells were washed with fresh media without 6TG and continuously cultured. VCN was analyzed by ddPCR assay (as described at Example 6). All 6 representative candidates demonstrated chemo-selection under treatment of 6TG. The results are illustrated in FIG. 20B.

### Example 8: In Vivo Mouse Experiments

In Vivo Mouse Experiments w/ NSG and/or Wasp^{-/-}

I) Mouse in Mouse Experiments

Mice

Male and female WASp-KO mice, CD45.2+, 5-10 weeks old are used as recipients and CD45.1⁺ WASp-KO will be used as donors or vice versa. C57BL/6J mice, CD45.1⁺, 6-10 weeks as controls. Donors may preferably be female.

All mice housed in, handled and experiments performed under sterile conditions.

Cell Preparation and Transplantation

In accordance with protocols described in “Singh et al. (2017). Molecular Therapy—Methods & Clinical Development, Volume 4, pp. 1-16,” donor mice will be sacrificed (according to the animal ethical guidelines) and lineage negative cells will be isolated using lineage cell depletion kit (at >95% purity). Donor cells will be seeded with stem cell factor (SCF) and thrombopoietin (TPO) and a two-hit lentiviral vector transduction will be performed. After transduction, cells will be washed to remove the virus and resuspended.

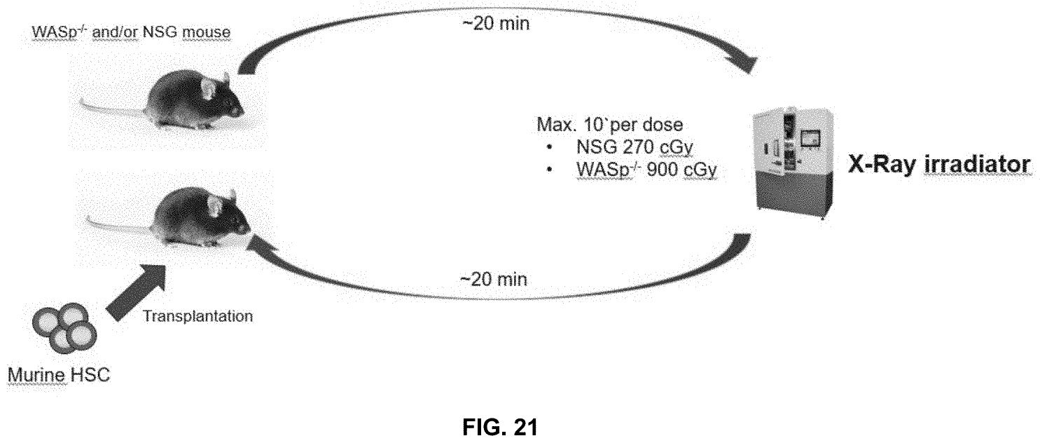

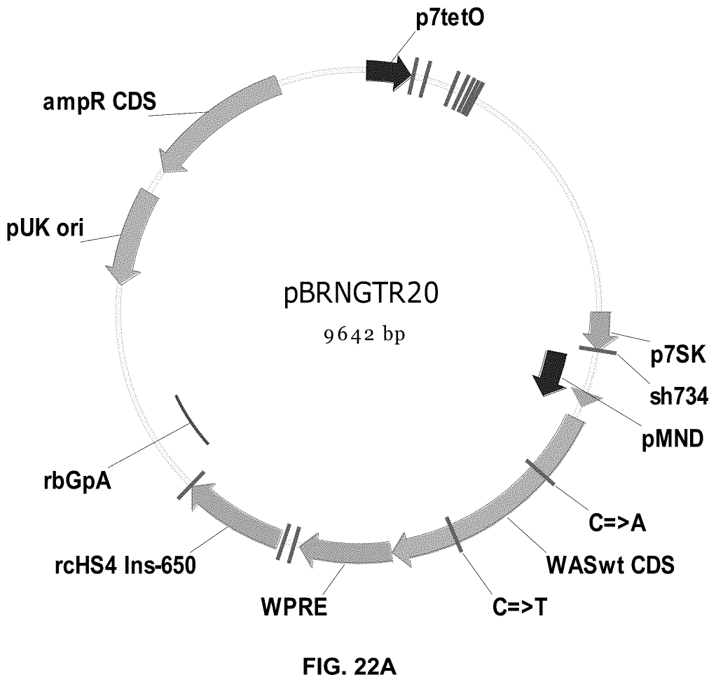

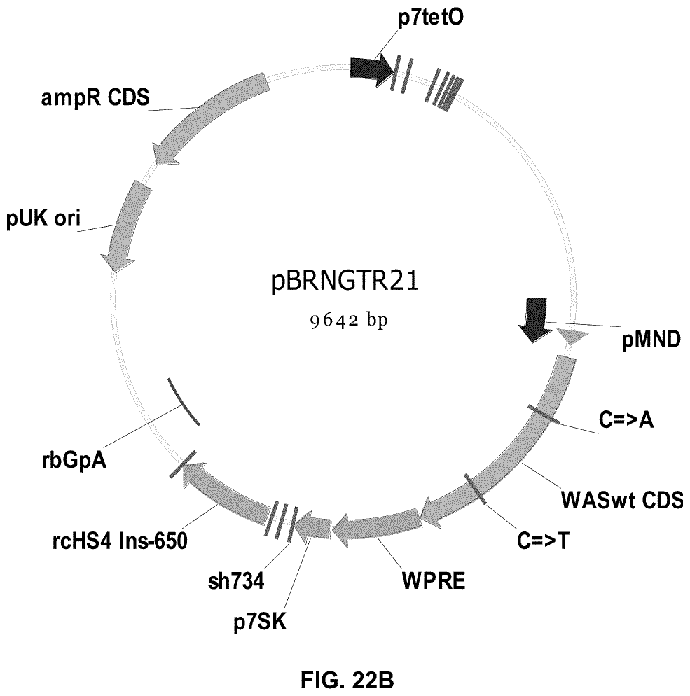

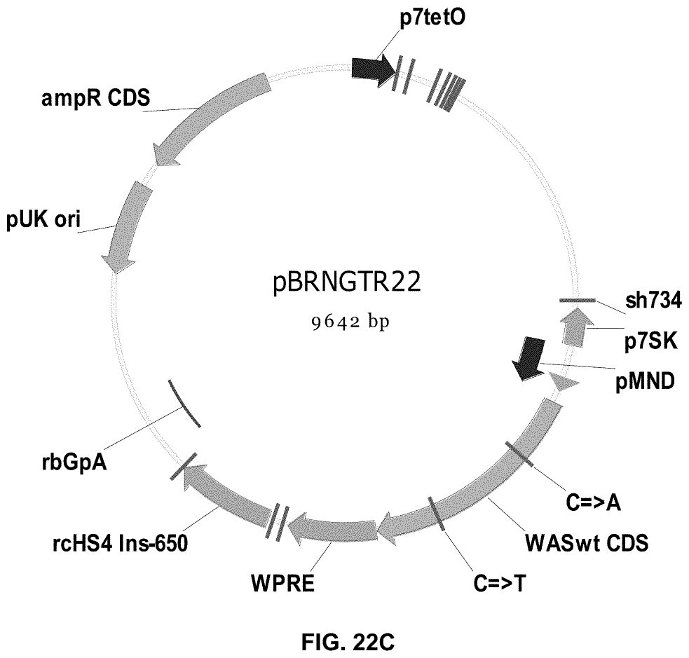

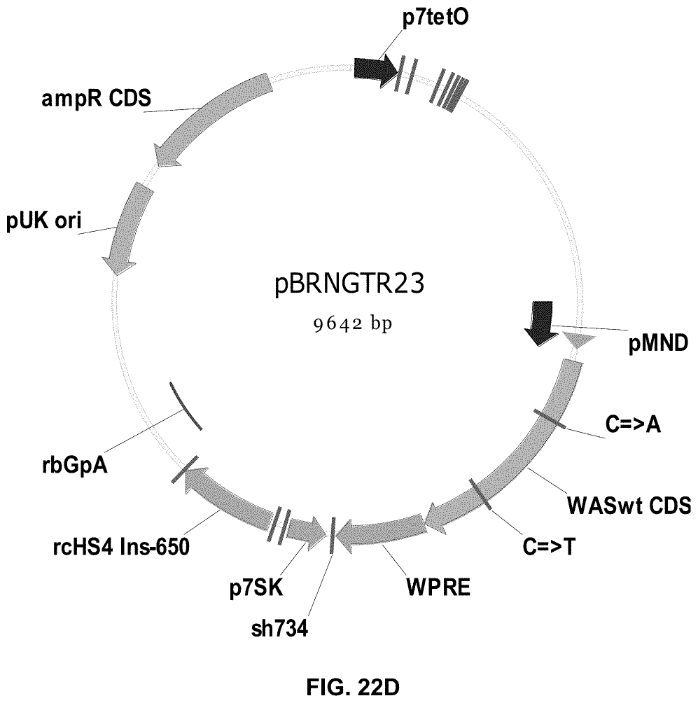

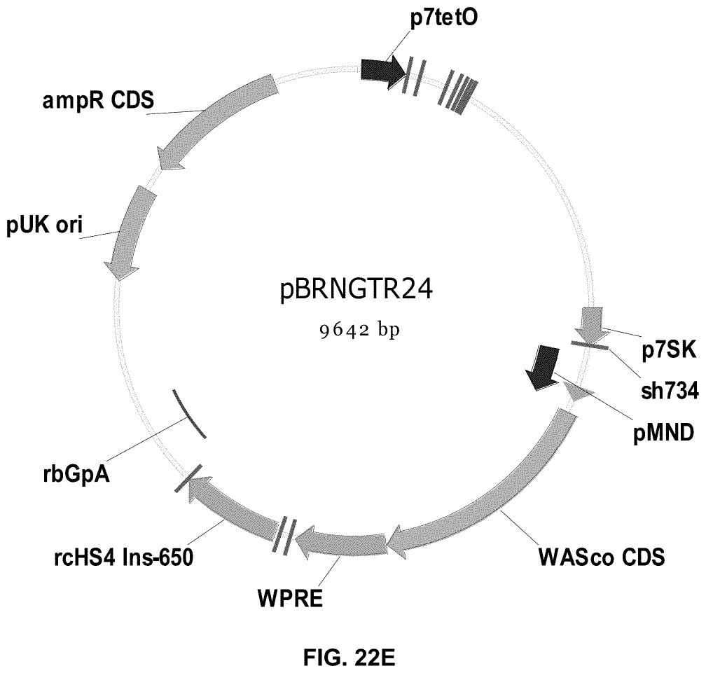

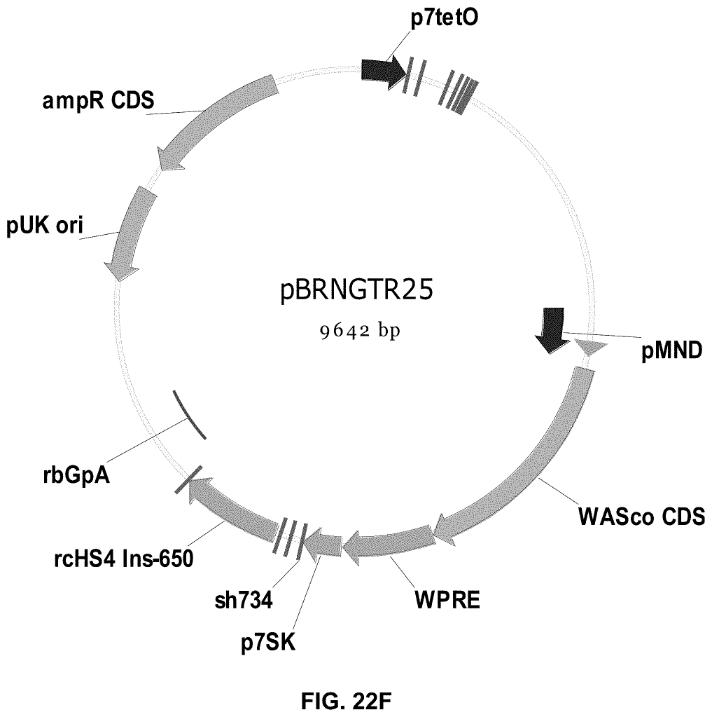

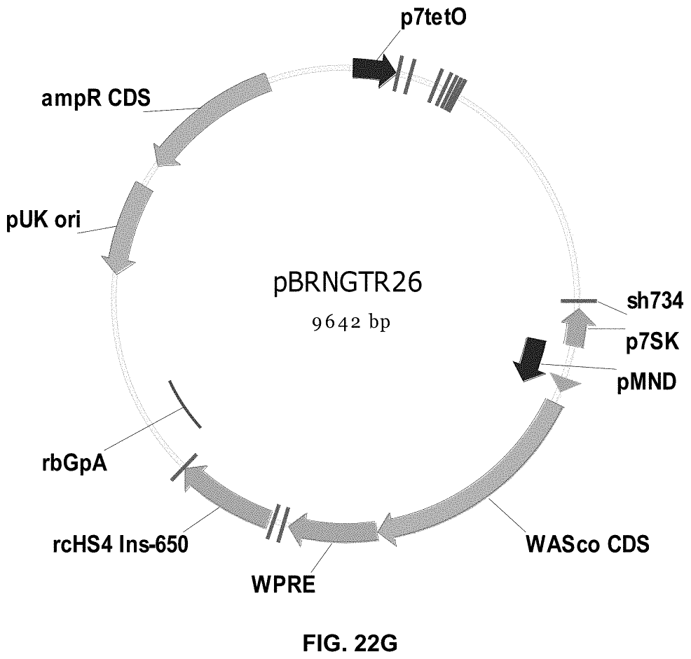

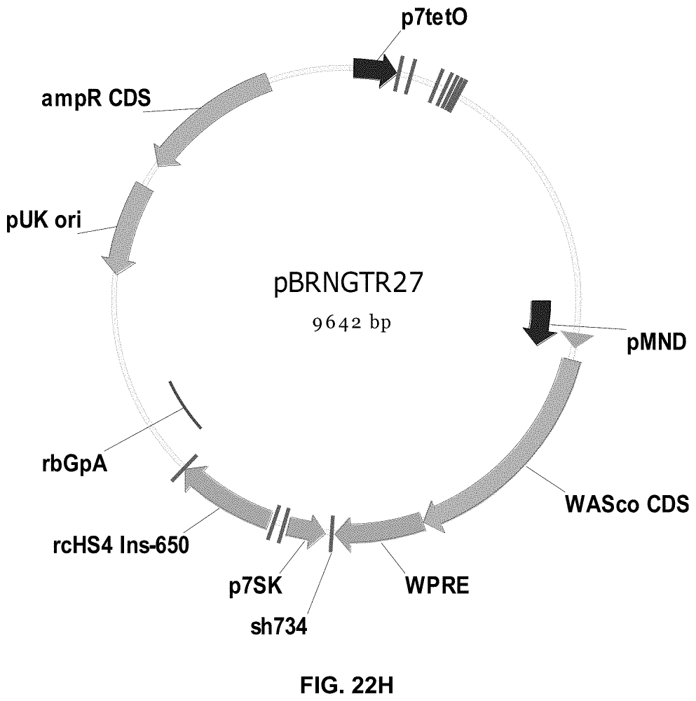

Recipient mice be irradiated using an X-Ray irradiator before being subjected to two rounds of 450 cGy irradiation with a gap of 4 hours between irradiations (see, for example, FIG. 21). After the second irradiation, mice will be transplanted with the prepared cells prepared (as described above) intravenously (i.v.) via tail vein using a cannula. Donor cell engraftment in the peripheral blood of the recipient mice will be analyzed using the strain specific markers (CD45.1 and/or CD45.2) at regular time points post-transplantation (i.e. 3, 6, 9, 12, 16 weeks post-transplantation). Analyses will include drawing peripheral blood by puncture of the vena saphena using heparin as anticoagulant. Blood will be lysed (to remove the red blood cells) and subjected to flow cytometry. Engraftment of the donor cells and engraftment of immune cell lineages (myeloid and lymphoid) will be analyzed. For long term analyses, mice will be analyzed 20 to 22 weeks post transplantation.

ii) Human in Mouse Experiments

Mice

NOD-SCID-IL-2rg (NSG) mice will be used. Mice will be housed and conditioned as described for Mouse in Mouse experiments above, except NSG mice will be irradiated once with a dose of 450 cGy using X-Ray irradiator.

Cell Preparation and Transplantation

G-CSF mobilized peripheral blood derived CD34+ cells will be purchased and stored (at −180° C.). Three days before transplantation, appropriate number of CD34+ cells will be thawed and the cell number will be determined. Cells will be seeded and preconditioned with human SCF, TPO and FLT3L for 48 hours. After preconditioning, cells will be transduced with LV for 16 hours. After transduction, cells will be washed to remove virus and transplanted. Mice will be transplanted with the transduced CD34+ cells intravenously (i.v.) via tail vein using a cannula. Engrafted human cells in the peripheral blood of recipient mice will be analyzed at regular intervals (at weeks 3, 6, 10, 12, and 16 post-transplantation). Mice will be sacrificed at week 16 and the tissues will be harvested to analyze human cell engraftment (including multi-lineage analyses). For long-term analyses, mice will be analyzed 20 to 22 weeks post transplantation.

### Additional Embodiments

In some embodiments is a composition including components which (i) introduce a gene encoding a Wiskott-Aldrich Syndrome protein (either wild-type or codon-optimized) into a hematopoietic stem cell (“HSC”), and (ii) decrease expression of HPRT in the HSC. In some embodiments, the components (e.g. nucleic acid sequences) are included within a lentiviral expression vector. In some embodiments, the lentiviral expression vector may be incorporated within a nanocapsule, such as one adapted to target HSCs. In some embodiments, the composition includes a lentiviral vector designed to effectuate expression of the Wiskott-Aldrich Syndrome protein under the control of an MND promoter.

In some embodiments is an expression vector including (i) a first nucleic acid sequence encoding an RNAi; and (ii) a second nucleic acid sequence encoding a Wiskott-Aldrich Syndrome protein (e.g. a wild-type protein or a codon-optimized protein). In some embodiments, the first nucleic acid encoding the RNAi encodes a small hairpin ribonucleic acid molecule (“shRNA”) targeting HPRT. In some embodiments, the first nucleic acid encoding the shRNA targeting the HPRT gene has a sequence having at least 80% identity to any one of SEQ ID NOS: 23-27. In some embodiments, the first nucleic acid sequence encoding the shRNA targeting the HPRT gene has a sequence having at least 90% identity to any one of SEQ ID NOS: 23-27. In some embodiments, the first nucleic acid sequence encoding the shRNA targeting the HPRT gene has a sequence having at least 95% identity to any one of SEQ ID NOS: 23-27. In some embodiments, the first nucleic acid sequence encoding the shRNA targeting the HPRT gene has a sequence having at least 97% identity to any one of SEQ ID NOS: 23-27. In some embodiments, the first nucleic acid sequence encoding the shRNA targeting the HPRT gene has a sequence of any one of SEQ ID NOS: 23-27.

In some embodiments, the second nucleic acid sequence encoding the Wiskott-Aldrich Syndrome protein has a sequence having at least 80% identity to any one of SEQ ID NOS: 1, 2, 3, and 4. In some embodiments, the second nucleic acid sequence encoding the Wiskott-Aldrich Syndrome protein has a sequence having at least 90% identity to any one of SEQ ID NOS: 1, 2, 3, and 4. In some embodiments, the second nucleic acid sequence encoding the Wiskott-Aldrich Syndrome protein has a sequence having at least 95% identity to any one of SEQ ID NOS: 1, 2, 3, and 4. In some embodiments, the second nucleic acid sequence encoding the Wiskott-Aldrich Syndrome protein has a sequence having at least 97% identity to any one of SEQ ID NOS: 1, 2, 3, and 4. In some embodiments, the second nucleic acid sequence encoding the Wiskott-Aldrich Syndrome protein has a sequence comprising any one of SEQ ID NOS: 1, 2, 3, and 4.

In some embodiments, the first nucleic acid sequence is operably linked to a Pol III promoter. In some embodiments, the Pol III promoter is a Homo sapiens cell-line HEK-293 7sk RNA promoter (see, for example, SEQ ID NO: 28). In some embodiments, the Pol III promoter is a 7sk promoter which includes a single mutation in its nucleic acid sequence as compared with SEQ ID NO: 28. In some embodiments, the Pol III promoter is a 7sk promoter which includes multiple mutations in its nucleic acid sequence as compared with SEQ ID NO: 28. In some embodiments, the Pol III promoter is a 7sk promoter which includes a deletion in its nucleic acid sequence as compared with SEQ ID NO: 28. In some embodiments, the Pol III promoter is a 7sk promoter which includes both a mutation and a deletion in its nucleic acid sequence as compared with SEQ ID NO: 28. In some embodiments, the first nucleic acid sequence is operably linked to promoter having at least 95% identity to that of SEQ ID NO: 28. In some embodiments, the first nucleic acid sequence is operably linked to promoter having at least 97% identity to that of SEQ ID NO: 28. In some embodiments, the first nucleic acid sequence is operably linked to promoter having at least 98% identity to that of SEQ ID NO: 28. In some embodiments, the first nucleic acid sequence is operably linked to promoter having at least 99% identity to that of SEQ ID NO: 28. In some embodiments, the first nucleic acid sequence is operably linked to a promoter having SEQ ID NO: 28.

In some embodiments is a vector comprising (i) a nucleic acid sequence encoding a micro-RNA based shRNA targeting a HPRT gene; and (ii) a nucleic acid sequence encoding WASP. In some embodiments, the second nucleic acid sequence encoding the Wiskott-Aldrich Syndrome protein has a sequence comprising any one of SEQ ID NOS: 1, 2, 3, and 4. In some embodiments, the nucleic acid sequence encoding the micro-RNA based shRNA targeting the HPRT gene has a sequence having at least 80% identity to any one of SEQ ID NOS: 19, 20, 21, and 22. In some embodiments, the nucleic acid sequence encoding the micro-RNA based shRNA targeting the HPRT gene has a sequence having at least 90% identity to any one of SEQ ID NOS: 19, 20, 21, and 22. In some embodiments, the nucleic acid sequence encoding the micro-RNA based shRNA targeting the HPRT gene has a sequence having at least 95% identity to any one of SEQ ID NOS: 19, 20, 21, and 22. In some embodiments, the nucleic acid sequence encoding the micro-RNA based shRNA targeting the HPRT gene has a sequence of any one of SEQ ID NOs: 19, 20, 21, and 22.

In some embodiments is a polynucleotide sequence including (a) a first portion encoding an shRNA targeting HPRT; (b) a second portion encoding a Wiskott-Aldrich Syndrome Protein. In some embodiments, the polynucleotide sequence further comprises (c) a third portion encoding a first promoter to drive expression of the sequence encoding the shRNA targeting HPRT; and (d) a fourth portion encoding a second promoter to drive expression of the sequence encoding WASP. In some embodiments, the polynucleotide sequence further comprises (e) a fifth portion encoding a central polypurine tract element; and (f) a sixth portion encoding a Rev response element (SEQ ID NO: 31). In some embodiments, the polynucleotide sequence further comprises a WPRE element (e.g. the WPRE element comprising SEQ ID NO: 41). In some embodiments, the polynucleotide sequence further comprises an insulator. In some embodiments, one or more insulators are incorporated into the polynucleotide sequence to enhance the safety profile of an expression vector and/or to improve transgene expression. In some embodiments, the insulator is a chromatin insulator. In some embodiments, the insulator has a nucleic acid sequence comprising any of SEQ ID NOS: 38, 39, and 40

In some embodiments are HSCs (e.g. CD34⁺ HSCs) which have been transduced with an expression vector including a Wiskott-Aldrich Syndrome protein transgene and an agent designed to reduce HPRT expression (e.g. an RNAi for knockdown of HPRT). In some embodiments, the transduced HSCs constitute a cell therapy product which may be administered to a subject in need of treatment thereof, e.g. for the treatment of the pathologies associated with Wiskott-Aldrich Syndrome or to alleviate the pathologies associated with Wiskott-Aldrich Syndrome.

In some embodiments are HSCs which have been transduced with an expression vector including a nucleic acid sequence encoding a Wiskott-Aldrich Syndrome protein and a nucleic acid encoding an anti-HPRT shRNA. In some embodiments, the anti-HPRT shRNA is driven by a 7sk promoter, and wherein the nucleic acid encoding a Wiskott-Aldrich Syndrome protein is driven by an MND promoter. In some embodiments, anti-HPRT shRNA driven by 7sk is oriented either upstream or downstream in the sense or anti-sense direction relative to a Wiskott-Aldrich Syndrome protein cassette (e.g. SEQ ID NO: 15). In some embodiments, the transduced HSCs constitute a cell therapy product which may be administered (such as in a pharmaceutical composition including a pharmaceutically acceptable vehicle) to a subject in need of treatment thereof, e.g. to treat or alleviate the pathologies associated with Wiskott-Aldrich Syndrome.

In some embodiments is a method of treating or alleviating pathologies associate with Wiskott-Aldrich Syndrome in a patient (e.g. a human patient) in need of treatment thereof comprising (a) transducing HSCs with a lentiviral expression vector, wherein the lentiviral expression vector includes a first nucleic acid sequence encoding an anti-HPRT shRNA or an anti-HPRT shRNA embedded within a microRNA; and a second nucleic acid sequence encoding a Wiskott-Aldrich Syndrome protein; and (b) transplanting the transduced HSCs within the patient. In some embodiments, the HSCs are autologous or allogeneic.

In some embodiments, the patient is pre-treated with myeloablative conditioning prior to the transplanting of the transduced HSCs administration (e.g. such as with a purine analog, including 6-thioguanine (“6TG”); with a chemotherapy agent; with radiation; with an antibody-drug conjugate, such as those described in US Patent Publication Nos. 2017/0360954 and 2018/0147294, and PCT Publication Nos. WO/2017/219025 and WO/2017/219029, the disclosures of which are each incorporated by reference herein in their entireties). In some embodiments, the transduced HSCs are selected for in vivo following the transplantation (e.g. such as with 6TG). In some embodiments, methotrexate or mycophenolic acid are administered to ameliorate any side effects of transplantation of the transduced HSCs (e.g. graft versus host disease).

In some embodiments is a pharmaceutical composition comprising a (a) a vector, such as an expression vector, including (i) a nucleic acid sequence encoding a shRNA targeting an HPRT gene; and (ii) a nucleic acid sequence encoding a Wiskott-Aldrich Syndrome Protein; and (b) a pharmaceutically acceptable carrier. In some embodiments, the pharmaceutical composition is formulated as an emulsion. In some embodiments, the pharmaceutical composition is formulated within micelles. In some embodiments, the pharmaceutical composition is encapsulated within a polymer. In some embodiments, the pharmaceutical composition is encapsulated within a liposome. In some embodiments, the pharmaceutical composition is encapsulated within minicells or nanocapsules.

In some embodiments is a stable producer cell line for generating viral titer, wherein the stable producer cell line is derived from one of a GPR, GPRG, GPRT, GPRGT, or GPRT-G packing cell line. In some embodiments, the stable producer cell line is derived from the GPRT-G cell line. In some embodiments, the stable producer cell line is generated by (a) synthesizing a vector by cloning nucleic acid sequences encoding an anti-HPRT shRNA and WASP into a recombinant plasmid (i.e. the synthesized vector may be any one of the vectors described herein); (b) generating DNA fragments from the synthesized vector; (c) forming a concatemeric array from (i) the generated DNA fragments from the synthesized vector, and (ii) from DNA fragments derived from an antibiotic resistance cassette plasmid; (d) transfecting one of the packaging cell lines with the formed concatemeric array; and (e) isolating the stable producer cell line. Additional methods of forming a stable producer cell line are disclosed in International Application No. PCT/US2016/031959, filed May 12, 2016, the disclosure of which is hereby incorporated by reference herein in its entirety.

Additional Embodiment 1. An expression vector comprising a first expression control sequence operably linked to a first nucleic acid sequence, the first nucleic acid sequence encoding a shRNA to knockdown HPRT; and a second expression control sequence operably linked to a second nucleic acid sequence, the second nucleic acid sequence encoding a Wiskott-Aldrich Syndrome protein.

Additional Embodiment 2. The expression vector of additional embodiment 1, wherein the shRNA comprises a hairpin loop sequence of SEQ ID NO: 32.

Additional Embodiment 3. The expression vector of additional embodiment 1, wherein the shRNA comprises the nucleic acid sequence of SEQ ID NO: 26.

Additional Embodiment 4. The expression vector of additional embodiment 1, wherein the shRNA has at least 95% sequence identity to a nucleic acid sequence selected from the group consisting of SEQ ID NO: 23, SEQ ID NO: 24, and SEQ ID NO: 25.

Additional Embodiment 5. The expression vector of additional embodiment 1, wherein the shRNA has at least 95% sequence identity to a nucleic acid sequence selected from the group consisting of SEQ ID NO: 34 and SEQ ID NO: 35.

Additional Embodiment 6. The expression vector of additional embodiment 1, wherein the shRNA has at least 95% sequence identity to a nucleic acid sequence selected from the group consisting of SEQ ID NO: 21 and SEQ ID NO: 22.

Additional Embodiment 7. The expression vector of additional embodiment 1, wherein the shRNA has at least 95% sequence identity to that of SEQ ID NO: 36.

Additional Embodiment 8. The expression vector of any one of the preceding additional embodiments, wherein the first expression control sequence comprises a Pol III promoter or a Pol II promoter.

Additional Embodiment 9. The expression vector of additional embodiment 8, wherein the Pol III promoter comprises 7sk.

Additional Embodiment 10. The expression vector of additional embodiment 9, wherein the 7sk promoter has a nucleic acid sequence having at least 95% sequence identity to that of SEQ ID NO: 28.

Additional Embodiment 11. The expression vector of additional embodiment 9, wherein the 7sk promoter has the nucleic acid sequence of SEQ ID NO: 28.

Additional Embodiment 12. The expression vector of additional embodiment 9, wherein the 7sk promoter has the nucleic acid sequence of SEQ ID NO: 29.

Additional Embodiment 13. The expression vector of any one of the preceding additional embodiments, wherein the second nucleic acid encodes a wild-type Wiskott-Aldrich Syndrome protein.

Additional Embodiment 14. The expression vector of any one of the preceding additional embodiments, wherein the second nucleic acid encodes a codon-optimized Wiskott-Aldrich Syndrome protein.

Additional Embodiment 15. The expression vector of any one of additional embodiments 1-12, wherein the second nucleic acid encoding the Wiskott-Aldrich Syndrome protein has a sequence having at least 95% identity to any one of SEQ ID NOS: 1, 2, 3, and 4.

Additional Embodiment 16. The expression vector of any one of additional embodiments 1-12, wherein the second nucleic acid encoding the Wiskott-Aldrich Syndrome protein has a sequence having at least 97% identity to any one of SEQ ID NOS: 1, 2, 3, and 4.

Additional Embodiment 17. The expression vector of any one of additional embodiments 1-12, wherein the second nucleic acid encoding the Wiskott-Aldrich Syndrome protein has a sequence having at least 99% identity to any one of SEQ ID NOS: 1, 2, 3, and 4.

Additional Embodiment 18. The expression vector of any one of additional embodiments 1-12, wherein the second nucleic acid encoding the Wiskott-Aldrich Syndrome protein has a sequence comprising any one of SEQ ID NOS: 1, 2, 3, and 4.

Additional Embodiment 19. The expression vector of any one of the preceding additional embodiments, wherein the second expression control sequence comprises an MND promoter.

Additional Embodiment 20. The expression vector of additional embodiment 19, wherein the MND promoter has a nucleic acid sequence having at least 95% identity to any one of SEQ ID NOS: 7, 8, 9, 10, 11, and 12.

Additional Embodiment 21. The expression vector of any one of the preceding additional embodiments, wherein the first expression control sequence operably linked to the first nucleic acid sequence is located downstream from the second expression control sequence operably linked to the second nucleic acid sequence.

Additional Embodiment 22. The expression vector of additional embodiment 21, wherein the first expression control sequence operably linked to the first nucleic acid sequence is oriented in the same direction as the second expression control sequence operably linked to the second nucleic acid sequence.

Additional Embodiment 23. The expression vector of additional embodiment 21, wherein the first expression control sequence operably linked to the first nucleic acid sequence is oriented in a first direction, wherein the second expression control sequence operably linked to the second nucleic acid sequence is oriented in a second direction, and where the first and second directions are opposite.

Additional Embodiment 24. The expression vector of any one of additional embodiments 1-20, wherein the first expression control sequence operably linked to the first nucleic acid sequence is located upstream from the second expression control sequence operably linked to the second nucleic acid sequence.

Additional Embodiment 25. The expression vector of additional embodiment 24, wherein the first expression control sequence operably linked to the first nucleic acid sequence is oriented in the same direction as the second expression control sequence operably linked to the second nucleic acid sequence.

Additional Embodiment 26. The expression vector of additional embodiment 24, wherein the first expression control sequence operably linked to the first nucleic acid sequence is oriented in a first direction, wherein the second expression control sequence operably linked to the second nucleic acid sequence is oriented in a second direction, and where the first and second directions are opposite.

Additional Embodiment 27. The expression vector of any one of additional embodiments 1-20, wherein the first expression control sequence operably linked to the first nucleic acid sequence is oriented in a first direction, wherein the second expression control sequence operably linked to the second nucleic acid sequence is oriented in a second direction, and where the first and second directions are opposite.

Additional Embodiment 28. The expression vector of additional embodiment 27, wherein the first expression control sequence operably linked to the first nucleic acid sequence is located downstream from the second expression control sequence operably linked to the second nucleic acid sequence.

Additional Embodiment 29. The expression vector of additional embodiment 27, wherein the first expression control sequence operably linked to the first nucleic acid sequence is located upstream from the second expression control sequence operably linked to the second nucleic acid sequence.

Additional Embodiment 30. The expression vector of any one of additional embodiments 1-20, wherein the first expression control sequence operably linked to the first nucleic acid sequence comprises is oriented in the same direction as the second expression control sequence operably linked to the second nucleic acid sequence.

Additional Embodiment 31. The expression vector of additional embodiment 30, wherein the first expression control sequence operably linked to the first nucleic acid sequence is located downstream from the second expression control sequence operably linked to the second nucleic acid sequence.

Additional Embodiment 32. The expression vector of additional embodiment 30, wherein the first expression control sequence operably linked to the first nucleic acid sequence is located upstream from the second expression control sequence operably linked to the second nucleic acid sequence.

Additional Embodiment 33. The expression vector of any one of additional embodiments 1-12, wherein the second nucleic acid sequence encodes a peptide comprising an amino acid sequence having at least 95% identity to any one of SEQS ID NOS: 5 and 6; and the first nucleic acid sequence encodes a nucleic acid molecule having at least 95% identity to SEQ ID NO: 16 or its complement thereof.

Additional Embodiment 34. The expression vector of any one of the preceding additional embodiments, wherein the expression vector further comprises an insulator selected from the group consisting of a 650cHS4 insulator, a 400cHS4 insulator, and a foamy virus insulator. Additional Embodiment 35. The expression vector of any one of the preceding additional embodiments, wherein the expression vector further comprises an insulator having a nucleic acid sequence selected from the group consisting of SEQ ID NO: 38, SEQ ID NO: 39, and SEQ ID NO: 40.

Additional Embodiment 36. The expression vector of any one of the preceding additional embodiments, wherein the expression vector is a lentiviral expression vector.

Additional Embodiment 37. The expression vector of additional embodiment 36, wherein the lentiviral expression vector is an integration defective lentiviral vector.

Additional Embodiment 38. An expression cassette comprising a nucleic acid sequence having at least 90% identity to that of SEQ ID NO: 15.

Additional Embodiment 39. The expression cassette of additional embodiment 38, wherein the nucleic acid sequence has at least 95% identity to that of SEQ ID NO: 15.

Additional Embodiment 40. A lentiviral expression vector comprising the expression cassette of any one of additional embodiments 38-39, and further comprising an insulator selected from the group consisting of a 650cHS4 insulator, a 400cHS4 insulator, and a foamy virus insulator.

Additional Embodiment 41. The lentiviral expression vector of additional embodiment 40, wherein the insulator has a nucleic acid sequence selected from the group consisting of SEQ ID NO: 38, SEQ ID NO: 39, and SEQ ID NO: 40.

Additional Embodiment 42. A host cell transduced with the expression vector of any one of additional embodiments 1-37 or the lentiviral expression vector of any one of additional embodiments 40-41.

Additional Embodiment 43. The host cell of additional embodiment 42, wherein the host cell is substantially HPRT deficient.

Additional Embodiment 44. The host cell of any one of additional embodiments 42-43, wherein the host cell expresses a Wiskott-Aldrich Syndrome protein.

Additional Embodiment 45. The host cell of any one of additional embodiments 42-44, wherein the host cell is formulated with a pharmaceutically acceptable carrier.

Additional Embodiment 46. The host cell of any one of additional embodiments 42-45, wherein the host cell is a hematopoietic stem cell.

Additional Embodiment 47. A host cell which is substantially HPRT deficient and which expresses a peptide comprising an amino acid sequence of any one of SEQ ID NOS: 5 and 6.

Additional Embodiment 48. The host cell of any one of additional embodiment 47, wherein the host cell is a hematopoietic stem cell.

Additional Embodiment 49. A host cell which is substantially HPRT deficient and which expresses a peptide having at least 95% identity to an amino acid sequence of any one of SEQ ID NOS: 5 and 6, wherein the host cell is prepared by transducing an HSC with an expression vector comprising a first expression control sequence operably linked to a first nucleic acid sequence, the first nucleic acid sequence encoding a shRNA to knockdown HPRT; and a second expression control sequence operably linked to a second nucleic acid sequence, the second nucleic acid sequence encoding a Wiskott-Aldrich Syndrome protein.

Additional Embodiment 50. The host cell of additional embodiment 49, wherein the second nucleic acid encodes a wild-type Wiskott-Aldrich Syndrome protein.

Additional Embodiment 51. The host cell of additional embodiment 49, wherein the second nucleic acid encodes a codon-optimized Wiskott-Aldrich Syndrome protein.

Additional Embodiment 52. The host cell of any one of additional embodiments 49-51, wherein the second expression control sequence is an MND promoter.

Additional Embodiment 53. The host cell of any one of additional embodiments 49-52, where the expression vector further comprises an insulator having a nucleic acid sequence comprising any one of SEQ ID NOS: 38, 39, and 40.

Additional Embodiment 54. The host cell of any one of additional embodiments 49-53, wherein the host cell is a hematopoietic stem cell.

Additional Embodiment 55. A pharmaceutical composition comprising the expression vector of any one of additional embodiments 1-37 or the lentiviral expression vector of any one of additional embodiments 40-41, and a pharmaceutically acceptable carrier.

Additional Embodiment 56. A pharmaceutical composition comprising the host cells of any one of additional embodiments 42-54.

Additional Embodiment 57. A method of selecting transduced cells comprising: transducing a population of cells with the expression vector of any one of additional embodiments 1-37 or the lentiviral expression vector of any one of additional embodiments 40-41; and enriching the population of transduced cells by selecting for the transduced cells with a purine analog.

Additional Embodiment 58. The method of additional embodiment 57, wherein the purine analog is selected from the group consisting of 6TG and 6-mercaptopurin.

Additional Embodiment 59. The method of any one of additional embodiments 57-58, wherein the transduced cells are HSCs.

Additional Embodiment 60. The method of additional embodiment 57, wherein the HSCs are allogenic HSCs.

Additional Embodiment 61. The method of additional embodiment 57, wherein the HSCs are autologous HSCs.

Additional Embodiment 62. The method of additional embodiment 57, wherein the HSCs are sibling matched HSCs.

Additional Embodiment 63. A method of alleviating pathologies associated with Wiskott-Aldrich Syndrome comprising administering a therapeutically effective amount of the host cells of any one of additional embodiments 42-54 to a patient in need of treatment thereof.

Additional Embodiment 64. The method of additional embodiment 63, wherein the pathologies associated with Wiskott-Aldrich Syndrome are selected from the group consisting of microthrombocytopenia, eczema, autoimmune diseases, and recurrent infections.

Additional Embodiment 65. The method of additional embodiment 63, wherein the recurrent infections include recurrent cutaneous infections.

Additional Embodiment 66. The method of additional embodiment 65, wherein the recurrent infections are selected from the group consisting of otitis media, skin abscess, pneumonia, enterocolitis, meningitis, sepsis, and urinary tract infection.

Additional Embodiment 67. The method of additional embodiment 64, wherein the eczema is treatment-resistant eczema.

Additional Embodiment 68. The method of additional embodiment 64, wherein the autoimmune diseases are selected from the group consisting of hemolytic anemia, vasculitis, arthritis, neutropenia, inflammatory bowel disease, and IgA nephropathy, Henoch-Schönlein-like purpura, dermatomyositis, recurrent angioedema, and uveitis.

Additional Embodiment 69. A polynucleotide comprising a first nucleic acid sequence having at least 95% sequence identity to that of SEQ ID NO: 14, and a second nucleic acid sequence having at least 95% sequence identity to that of SEQ ID NO: 15.

Additional Embodiment 70. The polynucleotide of additional embodiment 69, further comprising a nucleic acid sequence having SEQ ID NO: 13.

Additional Embodiment 71. The polynucleotide of additional embodiment 69, further comprising a nucleic acid sequence having SEQ ID NO: 41.

Additional Embodiment 72. The polynucleotide of additional embodiment 69, further comprising a nucleic acid sequence having SEQ ID NO: 31.

Additional Embodiment 73. The polynucleotide of additional embodiment 69, further comprising a nucleic acid sequence selected from the group consisting of SEQ ID NO: 38, SEQ ID NO: 39, and SEQ ID NO: 40.

Additional Embodiment 74. The polynucleotide of any one of additional embodiments 69-73, wherein the first nucleic acid sequence is located upstream of the second nucleic acid sequence.

Additional Embodiment 75. The polynucleotide of additional embodiment 74, wherein the first nucleic acid sequence comprises a same orientation as the second nucleic acid sequence.

Additional Embodiment 76. The polynucleotide of additional embodiment 75, wherein the same orientation is a forward orientation.

Additional Embodiment 77. The polynucleotide of additional embodiment 75, wherein the first nucleic acid sequence comprises a different orientation as the second nucleic acid sequence.

Additional Embodiment 78. The polynucleotide of additional embodiment 77, wherein the different orientation is a reverse orientation.

Additional Embodiment 79. The polynucleotide of any one of additional embodiments 69-73, wherein the first nucleic acid sequence is located downstream of the second nucleic acid sequence.

Additional Embodiment 80. The polynucleotide of additional embodiment 79, wherein the first nucleic acid sequence comprises a same orientation as the second nucleic acid sequence.

Additional Embodiment 81. The polynucleotide of additional embodiment 80, wherein the same orientation is a forward orientation.

Additional Embodiment 82. The polynucleotide of additional embodiment 79, wherein the first nucleic acid sequence comprises a different orientation as the second nucleic acid sequence.

Additional Embodiment 83. The polynucleotide of additional embodiment 82, wherein the different orientation is a reverse orientation.

Additional Embodiment 84. The polynucleotide of any one of additional embodiments 69-73, wherein the first nucleic acid sequence is oriented in a first direction, wherein the second nucleic acid sequence is oriented in a second direction, and where the first and second directions are opposite.

Additional Embodiment 85. The polynucleotide of additional embodiment 84, wherein the first nucleic acid sequence is located downstream from the second nucleic acid sequence.

Additional Embodiment 86. The polynucleotide of additional embodiment 84, wherein the first nucleic acid sequence is located upstream from the second nucleic acid sequence.

Additional Embodiment 87. The polynucleotide of any one of additional embodiments 69-73, wherein the first nucleic acid sequence is oriented in the same direction as the second nucleic acid sequence.

Additional Embodiment 88. The polynucleotide of additional embodiment 87, wherein the first nucleic acid sequence is located downstream from the second nucleic acid sequence.

Additional Embodiment 89. The polynucleotide of additional embodiment 87, wherein the first nucleic acid sequence is located upstream from the second nucleic acid sequence.

Additional Embodiment 90. A polynucleotide comprising a nucleic acid sequence having at least 90% identity to any one of SEQ ID NOS: 42-57.

Additional Embodiment 91. The polynucleotide of additional embodiment 90, wherein the nucleic acid sequence has at least 95% identity to any one of SEQ ID NOS: 42-57.

Additional Embodiment 92. The polynucleotide of additional embodiment 90, wherein the nucleic acid sequence has at least 97% identity to any one of SEQ ID NOS: 42-57.

Additional Embodiment 93. The polynucleotide of additional embodiment 90, wherein the nucleic acid sequence has at least 98% identity to any one of SEQ ID NOS: 42-57.

Additional Embodiment 94. The polynucleotide of additional embodiment 90, wherein the nucleic acid sequence has at least 99% identity to any one of SEQ ID NOS: 42-57.

Additional Embodiment 95. A polynucleotide having any one of SEQ ID NOS: 42-57.

Additional Embodiment 96. An expression vector comprising (a) a nucleic acid sequence encoding pTL20c; (b) a nucleic acid encoding a WASP expression cassette; and (c) a nucleic acid encoding a 7sk/sh734 expression cassette.

Additional Embodiment 97. The expression vector of additional embodiment 96, further comprising a nucleic acid sequence encoding an insulator.

Additional Embodiment 98. The expression vector of additional embodiment 96, wherein the WASP expression cassette is located upstream of the 7sk/sh734 expression cassette.

Additional Embodiment 99. The expression vector of additional embodiment 98, wherein the expression vector has a nucleic acid sequence having at least 90% identity to any one of one SEQ ID NOS: 44, 45, 48, and 49.

Additional Embodiment 100. The expression vector of additional embodiment 98, wherein the expression vector has a nucleic acid sequence having at least 95% identity to any one of one SEQ ID NOS: 44, 45, 48, and 49.

Additional Embodiment 101. The expression vector of additional embodiment 98, wherein the expression vector has a nucleic acid sequence having at least 90% identity to any one of one SEQ ID NOS: 51, 53, 55, and 57.

Additional Embodiment 102. The expression vector of additional embodiment 98, wherein the expression vector has a nucleic acid sequence having at least 95% identity to any one of one SEQ ID NOS: 51, 53, 55, and 57.

Additional Embodiment 103. The expression vector of additional embodiment 96, wherein the WASP expression cassette is located upstream of the 7sk/sh734 expression cassette, and wherein the 7sk/sh734 expression cassette comprises a reverse orientation relative to the WASP expression cassette.

Additional Embodiment 104. The expression vector of additional embodiment 103, wherein the expression vector has a nucleic acid sequence having at least 90% identity to any one of one SEQ ID NOS: 45, 49, 53, 57.

Additional Embodiment 105. The expression vector of additional embodiment 103, wherein the expression vector has a nucleic acid sequence having at least 95% identity to any one of one SEQ ID NOS: 45, 49, 53, 57.

Additional Embodiment 106. The expression vector of additional embodiment 96, wherein the WASP expression cassette is located upstream of the 7sk/sh734 expression cassette, and wherein the 7sk/sh734 expression cassette and the WASP expression cassette are oriented in the same direction.

Additional Embodiment 107. The expression vector of additional embodiment 106, wherein the expression vector has a nucleic acid sequence having at least 90% identity to any one of one SEQ ID NOS: 44, 48, 51, 55.

Additional Embodiment 108. The expression vector of additional embodiment 106, wherein the expression vector has a nucleic acid sequence having at least 95% identity to any one of one SEQ ID NOS: 44, 48, 51, 55.

Additional Embodiment 109. The expression vector of additional embodiment 96, wherein the WASP expression cassette is located downstream of the 7sk/sh734 expression cassette.

Additional Embodiment 110. The expression vector of additional embodiment 109, wherein the expression vector has a nucleic acid sequence having at least 90% identity to any one of one SEQ ID NOS: 42, 43, 46, and 47.

Additional Embodiment 111. The expression vector of additional embodiment 109, wherein the expression vector has a nucleic acid sequence having at least 95% identity to any one of one SEQ ID NOS: 42, 43, 46, and 47.

Additional Embodiment 112. The expression vector of additional embodiment 109, wherein the expression vector has a nucleic acid sequence having at least 90% identity to any one of one SEQ ID NOS: 50, 52, 54, and 56.

Additional Embodiment 113. The expression vector of additional embodiment 109, wherein the expression vector has a nucleic acid sequence having at least 95% identity to any one of one SEQ ID NOS: 50, 52, 54, and 56.

Additional Embodiment 114. The expression vector of additional embodiment 96, wherein the WASP expression cassette is located downstream of the 7sk/sh734 expression cassette, and wherein the 7sk/sh734 expression cassette comprises a reverse orientation relative to the WASP expression cassette.

Additional Embodiment 115. The expression vector of additional embodiment 114, wherein the expression vector has a nucleic acid sequence having at least 90% identity to any one of one SEQ ID NOS: 43, 47, 52, and 56.

Additional Embodiment 116. The expression vector of additional embodiment 114, wherein the expression vector has a nucleic acid sequence having at least 95% identity to any one of one SEQ ID NOS: 43, 47, 52, and 56.

Additional Embodiment 117. The expression vector of additional embodiment 96, wherein the WASP expression cassette is located downstream of the 7sk/sh734 expression cassette, and wherein the 7sk/sh734 expression cassette and the WASP expression cassette are oriented in the same direction.

Additional Embodiment 118. The expression vector of additional embodiment 117, wherein the expression vector has a nucleic acid sequence having at least 90% identity to any one of one SEQ ID NOS: 42, 46, 50, and 54.

Additional Embodiment 119. The expression vector of additional embodiment 117, wherein the expression vector has a nucleic acid sequence having at least 95% identity to any one of one SEQ ID NOS: 42, 46, 50, and 54.

Additional Embodiment 120. Use of an expression vector of any one of additional embodiments 96-119 for the preparation of a pharmaceutical composition for the treatment of Wiskott-Aldrich Syndrome, the pharmaceutical composition comprising host cells and a pharmaceutically acceptable carrier or excipient.

Additional Embodiment 121. A polynucleotide comprising a nucleic acid sequence having at least 90% identity to SEQ ID NO: 58.

Additional Embodiment 122. The polynucleotide of additional embodiment 121, wherein the nucleic acid sequence has at least 95% identity to SEQ ID NO: 58.

Additional Embodiment 123. The polynucleotide of additional embodiment 121, wherein the nucleic acid sequence has at least 96% identity to SEQ ID NO: 58.

Additional Embodiment 124. The polynucleotide of additional embodiment 121, wherein the nucleic acid sequence has at least 97% identity to SEQ ID NO: 58.

Additional Embodiment 125. The polynucleotide of additional embodiment 121, wherein the nucleic acid sequence has at least 98% identity to SEQ ID NO: 58.

Additional Embodiment 126. The polynucleotide of additional embodiment 121, wherein the nucleic acid sequence has at least 99% identity to SEQ ID NO: 58.

Additional Embodiment 127. A polynucleotide having SEQ ID NO: 58.

Additional Embodiment 128. A polynucleotide comprising a nucleic acid sequence having at least 90% identity to SEQ ID NO: 59.

Additional Embodiment 129. The polynucleotide of additional embodiment 128, wherein the nucleic acid sequence has at least 95% identity to SEQ ID NO: 59.

Additional Embodiment 130. The polynucleotide of additional embodiment 128, wherein the nucleic acid sequence has at least 96% identity to SEQ ID NO: 59.

Additional Embodiment 131. The polynucleotide of additional embodiment 128, wherein the nucleic acid sequence has at least 97% identity to SEQ ID NO: 59.

Additional Embodiment 132. The polynucleotide of additional embodiment 128, wherein the nucleic acid sequence has at least 98% identity to SEQ ID NO: 59.

Additional Embodiment 133. The polynucleotide of additional embodiment 128, wherein the nucleic acid sequence has at least 99% identity to SEQ ID NO: 59.

Additional Embodiment 134. A polynucleotide having SEQ ID NO: 59.

Additional Embodiment 135. A polynucleotide comprising a nucleic acid sequence having at least 90% identity to any one of SEQ ID NOS: 63 and 65.

Additional Embodiment 136. The polynucleotide of additional embodiment 135, wherein the nucleic acid sequence has at least 95% identity to any one of SEQ ID NOS: 63 and 65.

Additional Embodiment 137. The polynucleotide of additional embodiment 135, wherein the nucleic acid sequence has at least 96% identity to any one of SEQ ID NOS: 63 and 65.

Additional Embodiment 138. The polynucleotide of additional embodiment 135, wherein the nucleic acid sequence has at least 97% identity to any one of SEQ ID NOS: 63 and 65.

Additional Embodiment 139. The polynucleotide of additional embodiment 135, wherein the nucleic acid sequence has at least 98% identity to any one of SEQ ID NOS: 63 and 65.

Additional Embodiment 140. The polynucleotide of additional embodiment 135, wherein the nucleic acid sequence has at least 99% identity to any one of SEQ ID NOS: 63 and 65.

Additional Embodiment 141. A polynucleotide having any one of SEQ ID NOS: 63 and 65.

Additional Embodiment 142. A polynucleotide comprising a nucleic acid sequence having at least 90% identity to any one of SEQ ID NOS: 64 and 66.

Additional Embodiment 143. The polynucleotide of additional embodiment 142, wherein the nucleic acid sequence has at least 95% identity to any one of SEQ ID NOS: 64 and 66.

Additional Embodiment 144. The polynucleotide of additional embodiment 142, wherein the nucleic acid sequence has at least 96% identity to any one of SEQ ID NOS: 64 and 66.

Additional Embodiment 145. The polynucleotide of additional embodiment 142, wherein the nucleic acid sequence has at least 97% identity to any one of SEQ ID NOS: 64 and 66.

Additional Embodiment 146. The polynucleotide of additional embodiment 142, wherein the nucleic acid sequence has at least 98% identity to any one of SEQ ID NOS: 64 and 66.

Additional Embodiment 147. The polynucleotide of additional embodiment 142, wherein the nucleic acid sequence has at least 99% identity to any one of SEQ ID NOS: 64 and 66.

Additional Embodiment 148. A polynucleotide having any one of SEQ ID NOS: 64 and 66.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. An expression vector comprising a first expression control sequence operably linked to a first nucleic acid sequence, the first nucleic acid sequence encoding a shRNA to knockdown HPRT; and a second expression control sequence operably linked to a second nucleic acid sequence, the second nucleic acid sequence encoding a Wiskott-Aldrich Syndrome protein.

2. The expression vector of claim 1, wherein the shRNA has at least 95% sequence identity to a nucleic acid sequence selected from the group consisting of SEQ ID NO: 16, SEQ ID NO: 26, SEQ ID NO: 23, SEQ ID NO: 24, and SEQ ID NO: 25.

3. The expression vector of claim 1, wherein the first expression control sequence comprises a Pol III promoter or a Pol II promoter.

4. The expression vector of claim 3, wherein the Pol III promoter comprises a 7sK promoter, a mutated 7sk promoter, an H1 promoter, or an EF1a promoter.

5. The expression vector of claim 4, wherein the 7sk promoter comprises at least 95% sequence identity to one of SEQ ID NO: 28 or SEQ ID NO: 29.

6. The expression vector of claim 1, wherein the second nucleic acid sequence encodes a wild-type Wiskott-Aldrich Syndrome protein or a codon-optimized Wiskott-Aldrich Syndrome protein.

7. The expression vector of claim 6, wherein the second nucleic acid sequence encoding the Wiskott-Aldrich Syndrome protein has a sequence having at least 95% identity to any one of SEQ ID NOS: 1, 2, 3, and 4.

8. The expression vector of claim 1, wherein the second expression control sequence comprises an MND promoter.

9. The expression vector of claim 8, wherein the MND promoter has a nucleic acid sequence having at least 95% identity to any one of SEQ ID NOS: 7, 8, 9, 10, 11, and 12.

10. The expression vector of claim 1, wherein
the first expression control sequence operably linked to the first nucleic acid sequence is located downstream from the second expression control sequence operably linked to the second nucleic acid sequence; or
the first expression control sequence operably linked to the first nucleic acid sequence is located upstream from the second expression control sequence operably linked to the second nucleic acid sequence.

11. The expression vector of claim 1, wherein
the first expression control sequence operably linked to the first nucleic acid sequence is oriented in the same direction as the second expression control sequence operably linked to the second nucleic acid sequence; or
the first expression control sequence operably linked to the first nucleic acid sequence is oriented in a first direction, wherein the second expression control sequence operably linked to the second nucleic acid sequence is oriented in a second direction, and where the first and second directions are opposite.

12. The expression vector of claim 1, wherein the second nucleic acid sequence encodes a peptide comprising an amino acid sequence having at least 95% identity to any one of SEQS ID NOS: 5 and 6; and the first nucleic acid sequence encodes a nucleic acid molecule having at least 95% identity to SEQ ID NO: 16 or its complement thereof.

13. The expression vector of claim 1, further comprising an insulator selected from the group consisting of a 650cHS4 insulator, a 400cHS4 insulator, and a foamy virus insulator.

14. The expression vector of claim 13, wherein the insulator has at least 95% sequence identity to a nucleic acid sequence selected from the group consisting of SEQ ID NO: 38, SEQ ID NO: 39, and SEQ ID NO: 40.

15. The expression vector of any claim 1, wherein the expression vector is a lentiviral expression vector.

16. The expression vector of claim 1, wherein the expression vector comprises a nucleic acid sequence having at least 90% identity to any one of SEQ ID NOS: 42-57.

17. The expression vector of claim 1, wherein the expression vector comprises a nucleic acid sequence having at least 95% identity to any one of SEQ ID NOS: 42-57.

18. The expression vector of claim 1, wherein the expression vector comprises a nucleic acid sequence having at least 97% identity to any one of SEQ ID NOS: 42-57.

19. The expression vector of claim 1, wherein the expression vector comprises a nucleic acid sequence having at least 99% identity to any one of SEQ ID NOS: 42-57.

20. The expression vector of claim 1, wherein the expression vector comprises a nucleic acid sequence having any one of SEQ ID NOS: 42-57.

21. A host cell transduced with the expression vector of claim 1.

22. The host cell of claim 21, wherein the host cell is substantially HPRT deficient.

23. The host cell of claim 21, wherein the host cell expresses a Wiskott-Aldrich Syndrome protein.

24. The host cell of claim 21, wherein the host cell is a hematopoietic stem cell (HSC).

25. A host cell which is substantially HPRT deficient and which expresses a peptide having at least 95% identity to an amino acid sequence having any one of SEQ ID NOS: 5 and 6, wherein the host cell is prepared by transducing an HSC with an expression vector comprising a first expression control sequence operably linked to a first nucleic acid sequence, the first nucleic acid sequence encoding a shRNA to knockdown HPRT; and a second expression control sequence operably linked to a second nucleic acid sequence, the second nucleic acid sequence encoding a Wiskott-Aldrich Syndrome protein.

26. A method of treating or alleviating pathologies associated with Wiskott-Aldrich Syndrome comprising administering a therapeutically effective amount of the host cells of claim 25 to a patient in need of treatment thereof.

27. A method of selecting for transduced cells comprising: (i) transducing a population of cells with an expression vector comprising a first expression control sequence operably linked to a first nucleic acid sequence, the first nucleic acid sequence encoding a shRNA to knockdown HPRT; and a second expression control sequence operably linked to a second nucleic acid sequence, the second nucleic acid sequence encoding a Wiskott-Aldrich Syndrome protein; and (ii) enriching the population of transduced cells by selecting for the transduced cells with a purine analog, wherein the transduced cells are HSCs.

28. The method of claim 27, wherein the purine analog is selected from the group consisting of 6-Thioguanine (6TG) and 6-mercaptopurin (6MP).

29. The method of claim 27, wherein the HSCs are selected from the group consisting of allogenic HSCs, autologous HSCs, and sibling matched HSCs.


SEQUENCE LISTING (69 sequences)

SEQ ID NO: 1 - DNA, 1844 bp, Artificial Sequence
  Synthetic Wild type WASp mRNA
tcctcttcttaccctgcacccagagcctcgccagagaagacaagggcagaaagcaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgagtggctgagttacttgctgccctgtgctcctccccgcaggacatggctccccctccacctgctctgtgcccaccctccactctcctcttccaggcccccaaccccccatttcttccccaccaacccctccaatgctgttatccctgcctggtcctcacactcacccaacaatcccaaggccctttttatacaaaaattctcagttctcttcactcaaggatttttaaagaaaaataaaagaattgtctttctgtctctctataaaaaaaaaaaaaaaa

SEQ ID NO: 2 - DNA, 1506 bp, Artificial Sequence
  Synthetic Coding region of wild type WASp mRNA
atgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgac

SEQ ID NO: 3 - DNA, 1521 bp, Artificial Sequence
  Synthetic Codon-optimized WASp cDNA
accgccgccatgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgattgataa

SEQ ID NO: 4 - DNA, 1506 bp, Artificial Sequence
  Synthetic Coding region of codon-optimized WASp cDNA
atgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgat

SEQ ID NO: 5 - protein, 502 aa, Artificial Sequence
  Synthetic Protein sequences translated from wild type WASp cDNA
MSGGPMGGRPGGRGAPAVQQNIPSTLLQDHENQRLFEMLGRKCLTLATAVVQLYLALPPGAEHWTKEHCGAVCFVKDNPQKSYFIRLYGLQAGRLLWEQELYSQLVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDD

SEQ ID NO: 6 - protein, 502 aa, Artificial Sequence; identical to SEQ ID NO: 5
  Synthetic WASp amino acid sequence (translated from the codon-optimized WASp cDNA)

SEQ ID NO: 7 - DNA, 551 bp, Artificial Sequence
  Synthetic MND Promoter Sequence including MMLV and MPSV sequences
atcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgacgcgtcatgccaccatg

SEQ ID NO: 8 - DNA, 399 bp, Artificial Sequence
  Synthetic MND Promoter Sequence
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 9 - DNA, 309 bp, Artificial Sequence
  Synthetic MND Promoter Sequence
gaacagagagacagcagaatatgggccaaacaggatatctgtggtaagcagttcctgccccgctcagggccaagaacagttggaacagcagaatatgggccaaacaggatatctgtggtaagcagttcctgccccgctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctc

SEQ ID NO: 10 - DNA, 459 bp, Artificial Sequence
  Synthetic MND Promoter Sequence including the translation initiation codon
gaacagagagacagcagaatatgggccaaacaggatatctgtggtaagcagttcctgccccgctcagggccaagaacagttggaacagcagaatatgggccaaacaggatatctgtggtaagcagttcctgccccgctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcaccatg

SEQ ID NO: 11 - DNA, 311 bp, Artificial Sequence
  Synthetic MND Promoter Sequence
gaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctc

SEQ ID NO: 12 - DNA, 424 bp, Artificial Sequence
  Synthetic MND Promoter Sequence including the translation initiation codon
gaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatccaccggtcgccaccatg

SEQ ID NO: 13 - DNA, 590 bp, Artificial Sequence
  Synthetic WPRE Sequence
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 14 - DNA, 299 bp, Artificial Sequence
  Synthetic 7sk/sh734 expression cassette sequence
accatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatcct

SEQ ID NO: 15 - DNA, 3051 bp, Artificial Sequence
  Synthetic WASp expression cassette
acgcgtgcgatcgcaccggtggatcctcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccaattcaagcgagaagacaagggcagccgccaccatgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgattgataatactagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctggtaaccatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgcgtacgcggccgc

SEQ ID NO: 16 - DNA, 46 bp, Artificial Sequence
  Synthetic shRNA targeting an HPRT gene
aggatatgcccttgactatttgtccgacatagtcaagggcatatcc

SEQ ID NO: 17 - DNA, 46 bp, Artificial Sequence
  Synthetic shRNA targeting an HPRT gene
tcctatacgggaactgataaacaggctgtatcagttcccgtatagg

SEQ ID NO: 18 - DNA, 6565 bp, Artificial Sequence
  Synthetic pTL20c Plasmid Sequence
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 19 - RNA, 111 nt, Artificial Sequence
  Synthetic miRNA734 de novo (RNA form)
acccguacauauuuuuguguagcucuaguuuauagucaagggcauauccuuguguuuuuuuugaaggauaugcccuugacuauaaacuagcgcuacacuuuuucgucuugu

SEQ ID NO: 20 - RNA, 111 nt, Artificial Sequence
  Synthetic miRNA211 de novo (RNA form)
acccguacauauuuuuguguagcucuaguuauaaaucaaggucauaaccuuguguuuuuuuugaagguuaugaccuugauuuauaacuagcgcuacacuuuuucgucuugu

SEQ ID NO: 21 - DNA, 166 bp, Artificial Sequence
  Synthetic miRNA211-3G
ccggatcaacgccctaggtttatgtttggatgaactgacatacgcgtatccgtcttttaaatcaaggtcataaccgtagtgaaatatatattaaacaggttatgaccttgatttaaaatacggtaacgcggaattcgcaactattttatcaattttttgcgtcgac

SEQ ID NO: 22 - DNA, 166 bp, Artificial Sequence
  Synthetic miRNA734-3G
ccggatcaacgccctaggtttatgtttggatgaactgacatacgcgtatccgtcttatagtcaagggcatatcctgtagtgaaatatatattaaacaaggatatgcccttgactataatacggtaacgcggaattcgcaactattttatcaattttttgcgtcgac

SEQ ID NO: 23 - DNA, 56 bp, Artificial Sequence
  Synthetic shHPRT 616
gcaggcagtataatccaaatacctgacccatatttggattatactgcctgcttttt

SEQ ID NO: 24 - DNA, 55 bp, Artificial Sequence
  Synthetic shHPRT 211
ggttatgaccttgatttatacctgacccatattaaatcaaggtcataaccttttt

SEQ ID NO: 25 - DNA, 56 bp, Artificial Sequence
  Synthetic shHPRT 734.1
gggatatgcccttgactaatacctgacccatattagtcaagggcatatcccttttt

SEQ ID NO: 26 - DNA, 52 bp, Artificial Sequence
  Synthetic shHPRT 734
aggatatgcccttgactatttgtccgacatagtcaagggcatatcctttttt

SEQ ID NO: 27 - RNA, 51 nt, Artificial Sequence
  Synthetic Modified sh734
aggauaugcccuugacuaugcccugacccagcauagucaagggcauauccu

SEQ ID NO: 28 - DNA, 248 bp, Artificial Sequence
  Synthetic Homo sapiens cell-line HEK-293 7SK RNA promoter region
tcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 29 - DNA, 248 bp, Artificial Sequence
  Synthetic Homo sapiens cell-line HEK-293 7SK RNA promoter region with mutation
tcgacgtgcagtcgggctactgccccacccatagtaccggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 30 - DNA, 3901 bp, Artificial Sequence
  Synthetic TL20 viral Backbone
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagct

SEQ ID NO: 31 - DNA, 769 bp, Artificial Sequence
  Synthetic REV response element
aggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccg

SEQ ID NO: 32 - DNA, 9 bp, Artificial Sequence
  Synthetic Hairpin loop sequence of sh734
ttgtccgac

SEQ ID NO: 33 - RNA, 9 nt, Artificial Sequence
  Synthetic hsa-miR-22 loop sequence
ccugaccca

SEQ ID NO: 34 - DNA, 111 bp, Artificial Sequence
  Synthetic miRNA734 de novo (DNA form)
acccgtacatatttttgtgtagctctagtttatagtcaagggcatatccttgtgttttttttgaaggatatgcccttgactataaactagcgctacactttttcgtcttgt

SEQ ID NO: 35 - DNA, 111 bp, Artificial Sequence
  Synthetic miRNA211 de novo (DNA form)
acccgtacatatttttgtgtagctctagttataaatcaaggtcataaccttgtgttttttttgaaggttatgaccttgatttataactagcgctacactttttcgtcttgt

SEQ ID NO: 36 - DNA, 42 bp, Artificial Sequence
  Synthetic miRNA 451 hairpin sequence
aaaccgttaccattactgagtttagtaatggtaatggttctc

SEQ ID NO: 37 - DNA, 43 bp, Artificial Sequence
  Synthetic Agosh 734 5-3
atagtcaagggcatatcctcaagaaggatatgcccttgactac

SEQ ID NO: 38 - DNA, 649 bp, Artificial Sequence
  Synthetic 650-bp cHS4 insulator sequence
acggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggcttgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgag

SEQ ID NO: 39 - DNA, 36 bp, Artificial Sequence
  Synthetic Foamy virus 36-bp insulator sequence
aagggagacatctagtgatataagtgtgaactacac

SEQ ID NO: 40 - DNA, 412 bp, Artificial Sequence
  Synthetic reverse chicken HS4 400 bp chromatin insulator
atccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcccc

SEQ ID NO: 41 - DNA, 476 bp, Artificial Sequence
  Synthetic cPPT sequence
attccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccata

SEQ ID NO: 42 - DNA, 9395 bp, Artificial Sequence
  Synthetic pTL20c_SK734fwd_MND_WAS_400
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 43 - DNA, 9395 bp, Artificial Sequence
  Synthetic pTL20c_SK734rev_MND_WAS_400
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgatacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 44 - DNA, 9395 bp, Artificial Sequence
  Synthetic pTL20c_MND_WAS_SK734fwd_400
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgaccggtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 45 - DNA, 9395 bp, Artificial Sequence
  Synthetic pTL20c_MND_WAS_SK734rev_400
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgataccggtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 46 - DNA, 9395 bp, Artificial Sequence
  Synthetic pTL20c_SK734fwd_MND_coWAS_400
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgattgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 47 - DNA, 9395 bp, Artificial Sequence
  Synthetic pTL20c_SK734rev_MND_coWAS_400
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgatacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgattgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 48 - DNA, 9395 bp, Artificial Sequence
  Synthetic pTL20c_MND_coWAS_SK734fwd_400
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgattgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgaccggtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 49 - DNA, 9395 bp, Artificial Sequence
  Synthetic pTL20c_MND_coWAS_SK734rev_400
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgattgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgataccggtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 50 - DNA, 9642 bp, Artificial Sequence
  Synthetic pBRNGTR20 pTL20c_SK734fwd_MND_WAS_650
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaaggccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 51 - DNA, 9642 bp, Artificial Sequence
  Synthetic pBRNGTR21_pTL20c_MND_WAS_SK734fwd_650
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgaccggtgcggccgcatcgatgccgtagtacctttaagaccaatgacttacaaggcagctgtagatcttagccactttttaaaagaaaaggggggactggaagggctaattcactcccaaagaagacaaggccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 52 - DNA, 9642 bp, Artificial Sequence
  Synthetic pBRNGTR22_pTL20c_SK734rev_MND_WAS_650
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgatacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaaggccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 53 - DNA, 9642 bp, Artificial Sequence
  Synthetic pBRNGTR23_pTL20c_MND_WAS_SK734rev_650
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgataccggtgcggccgcatcgatgccgtagtacctttaagaccaatgacttacaaggcagctgtagatcttagccactttttaaaagaaaaggggggactggaagggctaattcactcccaaagaagacaaggccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 54 - DNA, 9642 bp, Artificial Sequence
  Synthetic pBRNGTR24_pTL20c_SK734fwd_MND_coWAS_650
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgattgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaaggccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 55 - DNA, 9642 bp, Artificial Sequence
  Synthetic pBRNGTR25_pTL20c_MND_coWAS_SK734fwd_650
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgattgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgaccggtgcggccgcatcgatgccgtagtacctttaagaccaatgacttacaaggcagctgtagatcttagccactttttaaaagaaaaggggggactggaagggctaattcactcccaaagaagacaaggccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 56 - DNA, 9642 bp, Artificial Sequence
  Synthetic pBRNGTR26_pTL20c_SK734rev_MND_coWAS_650
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgatacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgattgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaaggccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 57 - DNA, 9642 bp, Artificial Sequence
  Synthetic pBRNGTR27_pTL20c_MND_coWAS_SK734rev_650
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgattgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgataccggtgcggccgcatcgatgccgtagtacctttaagaccaatgacttacaaggcagctgtagatcttagccactttttaaaagaaaaggggggactggaagggctaattcactcccaaagaagacaaggccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 58 - DNA, 3160 bp, Artificial Sequence
  Synthetic 7SK/sh734_MND/hWASWT_WPRE_7SK/sh734 cassette
aagcttcgaagcgatcgcacgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgaccggtgcggccgc

SEQ ID NO: 59 - DNA, 3161 bp, Artificial Sequence
  Synthetic r7SK/sh734R_MND/hWASCO_WPRE_r7SK/sh734 cassette
aagcttcgaagcgatcgcacgcgtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgatacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgattgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgataccggtgcggccgc

SEQ ID NO: 60 - DNA, 179 bp, Artificial Sequence
  Synthetic gBlock 1
gctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatc

SEQ ID NO: 61 - DNA, 189 bp, Artificial Sequence
  Synthetic gBlock 2
ctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaaggccccatcctcactgactccgtcctggagttgga

SEQ ID NO: 62 - DNA, 187 bp, Artificial Sequence
  Synthetic gBlock 3
gcatgctaaatactgcacgtcgataccggtgcggccgcatcgatgccgtagtacctttaagaccaatgacttacaaggcagctgtagatcttagccactttttaaaagaaaaggggggactggaagggctaattcactcccaaagaagacaaggccccatcctcactgactccgtcctggagttgga

SEQ ID NO: 63 - DNA, 9702 bp, Artificial Sequence
  Synthetic Int Vector 1
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgaccggtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 64 - DNA, 9703 bp, Artificial Sequence
  Synthetic Int Vector 2
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgatacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgattgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgataccggtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 65 - DNA, 9703 bp, Artificial Sequence
  Synthetic Int Vector 3
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgatacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgataccggtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 66 - DNA, 9702 bp, Artificial Sequence
  Synthetic Int Vector 4
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgattgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgaccggtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 67 - DNA, 1512 bp, Artificial Sequence
  Synthetic Wild Type hWAS cDNA
atgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataa

SEQ ID NO: 68 - DNA, 1512 bp, Artificial Sequence; identical to SEQ ID NO: 67
  Synthetic Wild Type hWAS with silent mutations

SEQ ID NO: 69 - DNA, 1512 bp, Artificial Sequence
  Synthetic Codon-optimized hWAS cDNA
atgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgattgataa